SEQUENCE LISTING (46 sequences)

SEQ ID NO: 1 - protein, 7 aa, Artificial Sequence
  anti-CD3epsilon antibody OKT3 CDRH1
GYTFTRY

SEQ ID NO: 2 - protein, 6 aa, Artificial Sequence
  anti-CD3epsilon antibody OKT3 CDRH2
NPSRGY

SEQ ID NO: 3 - protein, 10 aa, Artificial Sequence
  anti-CD3epsilon antibody OKT3 CDRH3
YYDDHYCLDY

SEQ ID NO: 4 - protein, 10 aa, Artificial Sequence
  anti-CD3epsilon antibody OKT3 CDRL1
SASSSVSYMN

SEQ ID NO: 5 - protein, 7 aa, Artificial Sequence
  anti-CD3epsilon antibody OKT3 CDRL2
DTSKLAS

SEQ ID NO: 6 - protein, 9 aa, Artificial Sequence
  anti-CD3epsilon antibody OKT3 CDRL3
QQWSSNPFT

SEQ ID NO: 7 - protein, 7 aa, Artificial Sequence
  anti-CD28 antibody 15E8 CDRH1
GFSLTSY

SEQ ID NO: 8 - protein, 5 aa, Artificial Sequence
  anti-CD28 antibody 15E8 CDRH2
WAGGS

SEQ ID NO: 9 - protein, 15 aa, Artificial Sequence
  anti-CD28 antibody 15E8 CDRH3
DKRAPGKLYYGYPDY

SEQ ID NO: 10 - protein, 15 aa, Artificial Sequence
  anti-CD28 antibody 15E8 CDRL1
RASESVEYYVTSLMQ

SEQ ID NO: 11 - protein, 7 aa, Artificial Sequence
  anti-CD28 antibody 15E8 CDRL2
AASNVES

SEQ ID NO: 12 - protein, 9 aa, Artificial Sequence
  anti-CD28 antibody 15E8 CDRL3
QQTRKVPST

SEQ ID NO: 13 - protein, 6 aa, Artificial Sequence
  anti-CD28 receptor antibody TGN1412 CDRH1
GYTFSY

SEQ ID NO: 14 - protein, 6 aa, Artificial Sequence
  anti-CD28 receptor antibody TGN1412 CDRH2
YPGNVN

SEQ ID NO: 15 - protein, 11 aa, Artificial Sequence
  anti-CD28 receptor antibody TGN1412 CDRH3
SHYGLDWNFDV

SEQ ID NO: 16 - protein, 10 aa, Artificial Sequence
  anti-CD28 receptor antibody TGN1412 CDRL1
HASQNIYVLN

SEQ ID NO: 17 - protein, 7 aa, Artificial Sequence
  anti-CD28 receptor antibody TGN1412 CDRL2
KASNLHT

SEQ ID NO: 18 - protein, 9 aa, Artificial Sequence
  anti-CD28 receptor antibody TGN1412 CDRL3
QQGQTYPYT

SEQ ID NO: 19 - protein, 173 aa, Unknown
  OX40L native ligand for CD134
MERVQPLEENVGNAARPRFERNKLLLVASVIQGLGLLLCFTYICLHFSALQVSHRYPRIQSIKVQFTEYKKEKGFILTSQKEDEIMKVQNYLISLKGYFSQEVNISLHYQKDEEPLFQLKKVRSVNSLMVASLTYKDKVYLNVTTDNTSLDDFHVNGGELILIHQNPGEFCVL

SEQ ID NO: 20 - protein, 254 aa, Unknown
  4-1BBL cytokine
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 21 - protein, 233 aa, Homo sapiens
AEPKSPDKTHTCPPCPAPPVAGPSVFLFPPKPKDTLMIARTPEVTCWVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 22 - protein, 46 aa, Homo sapiens
TTTPAPRPPTPAPTIASQPLSLRPEACRPAAGGAVHTRGLDFACDI

SEQ ID NO: 23 - protein, 20 aa, Homo sapiens
AEPKSPDKTHTCPPCPKDPK

SEQ ID NO: 24 - protein, 185 aa, Homo sapiens
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 25 - protein, 259 aa, Homo sapiens
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 26 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 28 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 29 - protein, 271 aa, Artificial Sequence
  membrane-IL7
MAHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 30 - protein, 256 aa, Artificial Sequence
  membrane-IL15
MGLVRRGARAGPRMPRGWTALCLLSLLPSGFMAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 31 - protein, 330 aa, Homo sapiens [sequence omitted]

SEQ ID NO: 32 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 511 aa, Vesiculovirus Cocal virus
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 34 - protein, 504 aa, Artificial Sequence
  Blinatumomab, CD19/CD3-bispecific antibody construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 35 - protein, 10 aa, Artificial Sequence
  Blinatumomab, CD19 CDRL1
QSVDYDGDSY

SEQ ID NO: 36 - protein, 3 aa, Artificial Sequence
  Blinatumomab, CD19 CDRL2
DAS

SEQ ID NO: 37 - protein, 9 aa, Artificial Sequence
  Blinatumomab, CD19 CDRL3
QQSTEDPWT

SEQ ID NO: 38 - protein, 8 aa, Artificial Sequence
  Blinatumomab, CD19 CDRH1
GYAFSSYW

SEQ ID NO: 39 - protein, 8 aa, Artificial Sequence
  Blinatumomab, CD19 CDRH2
IWPGDGDT

SEQ ID NO: 40 - protein, 17 aa, Artificial Sequence
  Blinatumomab, CD19 CDRH3
ARRETTTVGRYYYAMDY

SEQ ID NO: 41 - protein, 8 aa, Artificial Sequence
  Blinatumomab, CD3 CDRH1
GYTFTRYT

SEQ ID NO: 42 - protein, 8 aa, Artificial Sequence
  Blinatumomab, CD3 CDRH2
INPSRGYT

SEQ ID NO: 43 - protein, 12 aa, Artificial Sequence
  Blinatumomab, CD3 CDRH3
ARYYDDHYCLDY

SEQ ID NO: 44 - protein, 5 aa, Artificial Sequence
  Blinatumomab, CD3 CDRL1
SSVSY

SEQ ID NO: 45 - protein, 3 aa, Artificial Sequence
  Blinatumomab, CD3 CDRL2
DTS

SEQ ID NO: 46 - protein, 7 aa, Artificial Sequence
  Blinatumomab, CD3 CDRL3
QQWSSNP

(19) United States
(12) Patent Application Publication
(10) Pub. No.: US 2023/0348624 A1
(43) Pub. Date: Nov. 2, 2023

(54) BISPECIFIC TRANSDUCTION ENHANCER

(71) Applicant: Umoja Biopharma, Inc., Seattle, WA (US)

(72) Inventors: Andrew SCHARENBERG, Seattle, WA (US); Ryan CRISMAN, Denver, CO (US); Christopher NICOLAI, Seattle, WA (US); Alessandra SULLIVAN, Seattle, WA (US); Kathryn MICHELS, Seattle, WA (US); Byoung RYU, Mercer Island, WA (US); Shon GREEN, Seattle, WA (US); Laurie BEITZ, Seattle, WA (US); Susana HERNANDEZ LOPEZ, Seattle, WA (US)

(21) Appl. No.: 17/796,523

(22) PCT Filed: Jan. 27, 2021
(86) PCT No.: PCT/US2021/015267

§ 371 Date: Jul. 29, 2022

Related U.S. Application Data
(60) Provisional application No. 62/968,028, filed on Jan. 30, 2020.

(51) Int. Cl. C07K 16/46; C12N 15/86; A61P 37/02

(52) U.S. Cl. CPC C07K 16/468; C12N 15/86; A61P 37/02; C12N 2740/15043; C07K 2317/31

(57) ABSTRACT

Provided are compositions and methods for transducing immune cells in vivo where a multispecific antibody (e.g. a bispecific T-cell engager) is administered to render immune cells in the subject more transducible by a vector, such as a lentiviral vector.

### CROSS REFERENCE TO RELATED APPLICATIONS

This application claims the benefit of the U.S. Provisional Patent Application Ser. No. 62/968,028, filed Jan. 30, 2020, the content of which is herein incorporated by reference in its entirety.

### DESCRIPTION OF THE TEXT FILE SUBMITTED ELECTRONICALLY

The content of the text file submitted electronically herewith is incorporated herein by reference in its entirety: A computer readable format copy of the Sequence Listing (filename: UMOJ-004_01WO_SeqList_ST25.txt, date created: Jan. 27, 2021, file size: 39.7 kilobytes).

### FIELD

The disclosure relates generally to in vivo transduction of immune cells to treat cancer and/or hematological malignancy.

### BACKGROUND

Cellular therapy generally employs the transduction of immune cells ex vivo to generate a population of therapeutic cells to be introduced into the patient. For example, T cells from an autologous or allogenic source can be transduced ex vivo with a vector encoding a chimeric antigen receptor. The resulting CAR T-cells are then infused into the patient.

It would be desirable to instead generate therapeutic cells in vivo by delivering a vector to the patient. Current methodologies for in vivo transduction of immune cells suffer from low efficiency. The present disclosure provides compositions and methods related to in vivo transduction of immune cells to treat cancer and/or hematological malignancy.

### SUMMARY

The present disclosure provides methods of transducing immune cells in a subject in need thereof, comprising a) administering a multispecific antibody to render immune cells in the subject more transducible; and b) administering a vector, optionally a viral vector. In some embodiments, the vector is a lentiviral vector. In some embodiments, the method transduces the immune cells. In some embodiments, the immune cells are T cells. In some embodiments, the vector is a lentiviral vector.

In some embodiments, the multispecific antibody comprises a T-cell antigen-specific binding domain. In some embodiments, the T-cell antigen is CD3, CD4, CD8, or TCR. In some embodiments, the multispecific antibody comprises a second antigen-specific binding domain. In some embodiments, the second antigen is CD19. In some embodiments, the second antigen is CD19, EpCAM, Her2/neu, EGFR, CD66e, CD33, EphA2, or MCSP. In some embodiments, the second antigen is CD19, EpCAM, CD20, CD123, BCMA, B7-H3, CDE, or PSMA. In some embodiments, the second antigen is a myeloid cell or dendritic cell antigen. In some embodiments, the second antigen is CD33, DC-SIGN, CD11b, CD11c, or CD18. In some embodiments, the multispecific antibody is a bispecific antibody. In some embodiments, the bispecific antibody is a bispecific T-cell engager (BiTE). In some embodiments, the BiTE is a CD19×CD3 BiTE. In some embodiments, the CD19×CD3 BiTE is blinatumomab.

In some embodiments, the multispecific antibody activates the immune cells. In some embodiments, the multispecific antibody increases activation of the immune cells compared to administration of a vehicle control. In some embodiments, the multispecific antibody increases the number of immune cells in a lymph node of the subject. In some embodiments, the multispecific antibody increases transduction of the immune cells compared to administration of the viral vector alone. In some embodiments, the multispecific antibody enhances in vivo transduction of the immune cells by the viral vector. In some embodiments, the multispecific antibody reduces the effective concentration (EC50) of the viral vector. In some embodiments, the method achieves the same level of transduction of immune cells as a method comprising administering a higher concentration of the viral vector without administering the multispecific antibody.

In some embodiments, the vector is a viral vector comprising a polynucleotide encoding a T cell receptor or chimeric antigen receptor. In some embodiments, the chimeric antigen receptor is an anti-CD19 chimeric antigen receptor. In some embodiments, the vector is a viral vector comprising a polynucleotide encoding a cytokine receptor. In some embodiments, the cytokine receptor is a drug-inducible cytokine receptor. In some embodiments, the vector further comprises one or more transgenes. In some embodiments, the viral vector comprises the transgene encoding a TGFβ dominant negative receptor. In some embodiments, the lentiviral vector comprises one or more cell surface receptors that bind to a ligand on a target host cell, heterologous viral envelope glycoproteins, fusion glycoproteins, T-cell activation or co-stimulation molecules, ligands for CD19 or a functional fragment thereof, cytokines or cytokine-based transduction enhancers, and/or transmembrane proteins comprising a mitogenic domain and/or cytokine-based domain exposed on the surface and/or conjugated to the surface of the lentiviral vector. In some embodiments, the one or more T-cell activation or co-stimulation molecules comprise one or more T-cell ligands. In some embodiments, the lentiviral vector is pseudotyped with a Cocal virus envelop protein. In some embodiments, the lentiviral vector is pseudotyped with a Nipah virus envelop protein. In some embodiments, the Nipah envelop protein is engineered to bind EpCAM, CD4, or CD8.

In some embodiments, step a) and/or step b) of the methods of transducing immune cells comprises subcutaneous administration. In some embodiments, step a) and/or step b) comprises intralymphatic administration. In some embodiments, step a) or step b) comprises intravenous administration. In some embodiments, both step a) and step b) comprise intravenous administration. In some embodiments, the multispecific antibody is administered at a dose of about 0.001 mg/kg to about 1 mg/kg.

In some embodiments, the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.

The present disclosure provides methods of transducing immune cells in a subject in need thereof, comprising: a) administering a polynucleotide encoding a multispecific antibody to activate immune cells in the subject; and b) administering a vector, optionally a viral vector. In some embodiments, the method transduces the immune cells. In some embodiments, In some embodiments, the polynucleotide encoding a multispecific antibody is an RNA. In some embodiments, the immune cells are T cells. In some embodiments, the vector is a lentiviral vector. In some embodiments, the multispecific antibody comprises a T-cell antigen-specific binding domain. In some embodiments, the T-cell antigen is CD3, CD4, CD8, or TCR. In some embodiments, the multispecific antibody comprises a second antigen-specific binding domain. In some embodiments, the second antigen is CD19. In some embodiments, the second antigen is CD19, EpCAM, Her2/neu, EGFR, CD66e, CD33, EphA2, MCSP, CD22, CD79a, CD79b, or sIgM. In some embodiments, the second antigen is CD19, EpCAM, CD20, CD123, BCMA, B7-H3, CDE, or PSMA. In some embodiments, the second antigen is a lymph node antigen. In some embodiments, the multispecific antibody is a trispecific antibody. In some embodiments, the multispecific antibody is a bispecific antibody. In some embodiments, the bispecific antibody is a bispecific T-cell engager (BiTE). In some embodiments, the BiTE is a CD19×CD3 BiTE. In some embodiments, the CD19×CD3 BiTE is blinatumomab. In some embodiments, the multispecific antibody activates the immune cells. In some embodiments, the multispecific antibody increases activation of the immune cells compared to administration of a vehicle control. In some embodiments, the multispecific antibody increases the number of immune cells in a lymph node of the subject. In some embodiments, the multispecific antibody increases transduction of the immune cells compared to administration of the viral vector alone. In some embodiments, the multispecific antibody enhances in vivo transduction of the immune cells by the viral vector. In some embodiments, the multispecific antibody reduces the effective concentration (EC50) of the viral vector. In some embodiments, the method achieves the same level of transduction of immune cells as a method comprising administering a higher concentration of the viral vector without administering the multispecific antibody. In some embodiments, step a) and/or step b) comprises subcutaneous administration. In some embodiments, step a) and/or step b) comprises intralymphatic administration. In some embodiments, wherein step a) and/or step b) comprises intravenous administration. In some embodiments, the viral vector comprises a polynucleotide encoding a T cell receptor or chimeric antigen receptor. In some embodiments, the chimeric antigen receptor is an anti-CD19 chimeric antigen receptor. In some embodiments, the viral vector comprises a polynucleotide encoding a cytokine receptor. In some embodiments, the cytokine receptor is a drug-inducible cytokine receptor. In some embodiments, the lentiviral vector comprises one or more cell surface receptors that bind to a ligand on a target host cell, heterologous viral envelope glycoproteins, fusion glycoproteins, T-cell activation or co-stimulation molecules, ligands for CD19 or a functional fragment thereof, cytokines or cytokine-based transduction enhancers, and/or transmembrane proteins comprising a mitogenic domain and/or cytokine-based domain exposed on the surface and/or conjugated to the surface of the lentiviral vector. In some embodiments, the one or more T-cell activation or co-stimulation molecules comprise one or more T-cell ligands. In some embodiments, the vector further comprises one or more transgenes. In some embodiments, the viral vector comprises the transgene encoding a TGFβ dominant negative receptor. In some embodiments, the lentiviral vector is pseudotyped with a Cocal virus envelop protein. In some embodiments, the lentiviral vector is pseudotyped with a Nipah virus envelop protein. In some embodiments, the Nipah envelop protein is engineered to bind EpCAM, CD4, or CD8. In some embodiments, the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.

The present disclosure provides combination therapies for use in transducing immune cells in vivo, comprising a multispecific antibody and a vector, optionally a viral vector.

The present disclosure provides pharmaceutical compositions comprising a multispecific antibody and a vector, optionally a viral vector.

The present disclosure provides kits comprising 1) a multispecific antibody and 2) a vector, optionally a viral vector. The present disclosure also provides kits comprising 1) a polynucleotide encoding a multispecific antibody and 2) a vector, optionally a viral vector. In some embodiments, the kit of the disclosure is for use in: a) transducing immune cells in a subject in need thereof; and/or b) treating a disease or disorder in a subject in need thereof.

The present disclosure provides methods of treating a disease or disorder in a subject in need thereof, comprising: a) administering a multispecific antibody to activate immune cells in the subject; and b) before, after and concurrently with step a), administering a vector, optionally a viral vector. In some embodiments, the method transduces the immune cells. In some embodiments, the disease or disorder is a cancer. In some embodiments, the disease or disorder is a hematological malignancy. In some embodiments, the hematological malignancy is B cell lymphoma. In some embodiments, the method treats the disease or disorder faster than administering the multispecific antibody alone and/or the vector alone. In some embodiments, the method results in a better treatment outcome of the disease or disorder than administering the multispecific antibody alone and/or the vector alone. In some embodiments, multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor. In some embodiments, the method results in faster depletion of malignant B cells in the subject than administering the multispecific antibody alone and/or the vector alone. In some embodiments, the method results in lower number of residual malignant B cells and/or lower recurrence rate of the B cell lymphoma in the subject than administering the multispecific antibody alone and/or the vector alone.

### BRIEF DESCRIPTION OF THE DRAWINGS

FIG. 1 depicts an embodiment in which blinatumomab is co-administered with a viral vector.

FIG. 2A shows flow cytometry to measure CD25+ T cells in an experiment performed on cultured primary T cells in 50:50 ratio with B cells.

FIG. 2B shows flow cytometry to measure CD25+ T cells in an experiment performed on cultured primary T cells.

FIG. 3A shows flow cytometry to measure anti-CD19 chimeric antigen receptor expressing T cells in an experiment performed on cultured primary T cells in 50:50 ratio with B cells.

FIG. 3B shows flow cytometry to measure anti-CD19 chimeric antigen receptor expressing T cells in an experiment performed on cultured primary T cells.

FIG. 4 shows a graph of the ratio of B- to T-cells after culturing the cells in the presence of blinatumomab.

FIG. 5A shows flow panel validation and gating strategy for anti CD19CAR-TGFβ T cells generated and maintained in culture.

FIG. 5B shows flow panel validation and gating strategy for anti CD19CAR-TGFβ T cells generated in CD34 humanized mice.

FIG. 6 shows bar graphs of transiently activation of CD4+ (left) and CD8+ (right) T cells upon Blinatumomab administration.

FIG. 7 is a chart showing numbers of B cells over time upon lentivirus and/or Blinatumomab administration.

FIG. 8 shows representative flow cytometry plots of blood samples collected from mice on study day 33. The plots were gated on CD3+ live singlets. Intracellular anti-2A peptide staining was included into the panel as an alternative method for detecting CARs.

FIG. 9 shows representative flow cytometry plots, gated on live singlets, of spleen and bone marrow samples from the indicated study arms harvested on study day 52.

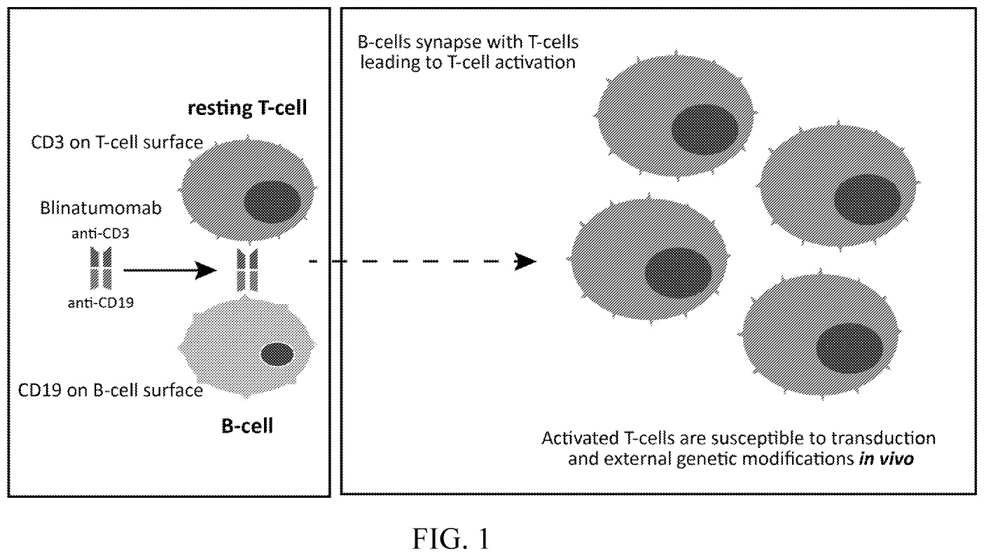

### DETAILED DESCRIPTION

The present disclosure provides compositions and methods related to the use of a multispecific antibody to facilitate the generation of genetically engineered target cells a vector. In some embodiments, the use of multispecific antibody improves the transduction efficiency of a vector in vivo. In some embodiments, transduction of target cells in a subject can be enhanced upon administering to the subject one or more multispecific antibodies before, concurrently with, or after administering the vector to the subject.

Without wishing to be bound to any particular theory, it is contemplated that a multispecific antibody according to the present disclosure may exert its effect through one or more mechanisms of action including but not limited to: (i) Stimulating the target cells (e.g. immune cells) to enter a more activated and/or more proliferative state. This may increase transduction efficiency of the vector. Viral vector entry and payload delivery are typically more efficiently when the target cells are in an active/proliferative state. (ii) Causing the immune cell to exit the Go phase of the cell cycle. (iii) Causing the immune cells to replicate at least once. (iv) Increasing the metabolic fitness of the immune cell. (v) Attracting increased numbers of immune cells to a physiologically relevant site (e.g. lymph node).

As a result, the methods and compositions described herein may be used to transduce significantly more cells and/or transduce the same number of cells at a lower effective concentrations of the vector. The compositions and methods of the disclosure may facilitate administering of the vectors directly into the subjects in need of treatment. Moreover, by decreasing the concentration at which the vector is effective in vivo, the compositions and methods of the disclosure may limit side effects due to vector toxicity or off-target transduction. Accordingly, the present disclosure provides a safer and more efficient gene therapy.

### Multispecific Antibody

The term multispecific antibody refers to an antibody molecule that has two or more antigen-binding domains, for example two (bispecific) or three (tri-specific) or four (tetra-specific) binding domains. In some embodiments, the multispecific antibody is a bispecific antibody. In some embodiments, the multispecific antibody is a trispecific antibody. In some embodiments, the multispecific antibody is a construct having more than three (e.g. four, five . . . ) specificities.

Multispecific antibody molecules of the present disclosure may be constructed from various antibody fragments known in the art. For example a diabody is a bispecific antibody molecule composed of a non-covalent dimer of ScFv fragments, while a F(ab′)2 is a bispecific antibody molecule composed of 2 Fab fragments linked by a hinge region. The skilled person will therefore be aware that different antibody fragments can be arranged in various combinations in order to produce a bi- or multi-specific antibody molecule.

Various multispecific and/or bispecific formats include recombinant IgG-like dual targeting molecules, wherein the two sides of the molecule each contain the Fab fragment or part of the Fab fragment of at least two different antibodies; IgG fusion molecules, wherein full length IgG antibodies are coupled to an extra Fab fragment or parts of Fab fragment; Fc fusion molecules, wherein single chain Fv molecules or stabilized diabodies are coupled to heavy-chain constant-domains, Fc-regions or parts thereof; Fab fusion molecules, wherein different Fab-fragments are coupled together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are coupled to each other or to another protein or carrier molecule, or multispecific antibodies generated by arm exchange. Exemplary multispecific and/or bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

Examples of trispecific or tetraspecific antibody formats include but are not limited to Fab3, triabody, tetrabody, tribody, DVD-Ig, JgG-scFv, ScFv2-Fc, tandAbs and DNL-Fab3.

In some embodiments, the multispecific antibody (e.g., bispecific antibody) of the disclosure comprises specific antigen-binding domains for a combination of two antigens selected from Table 1 below (each “X” mark indicates a combination):

TABLE 1

| | CD3 | CD4 | CD8 | CTLA-4 | TCR | CD16 | CD25 | CD28 | CD64 | CD89 | NKG2D | NKp46 |
|---|---|---|---|---|---|---|---|---|---|---|---|---|
| CD19 | X | X | X | X | X | X | X | X | X | X | X | X |
| EpCAM | X | X | X | X | X | X | X | X | X | X | X | X |
| CD20 | X | X | X | X | X | X | X | X | X | X | X | X |
| CD123 | X | X | X | X | X | X | X | X | X | X | X | X |
| BCMA | X | X | X | X | X | X | X | X | X | X | X | X |
| B7-H3 | X | X | X | X | X | X | X | X | X | X | X | X |
| CDE | X | X | X | X | X | X | X | X | X | X | X | X |
| PSMA | X | X | X | X | X | X | X | X | X | X | X | X |
| Her2/neu | X | X | X | X | X | X | X | X | X | X | X | X |
| EGFR | X | X | X | X | X | X | X | X | X | X | X | X |
| CD66e | X | X | X | X | X | X | X | X | X | X | X | X |
| CD33 | X | X | X | X | X | X | X | X | X | X | X | X |
| EphA2 | X | X | X | X | X | X | X | X | X | X | X | X |
| MCSP | X | X | X | X | X | X | X | X | X | X | X | X |
| CD22 | X | X | X | X | X | X | X | X | X | X | X | X |
| CD79a | X | X | X | X | X | X | X | X | X | X | X | X |
| CD79b | X | X | X | X | X | X | X | X | X | X | X | X |
| sIgM | X | X | X | X | X | X | X | X | X | X | X | X |
| DC- | X | X | X | X | X | X | X | X | X | X | X | X |
| SIGN | | | | | | | | | | | | |
| CD11b | X | X | X | X | X | X | X | X | X | X | X | X |
| CD11c | X | X | X | X | X | X | X | X | X | X | X | X |
| CD18 | X | X | X | X | X | X | X | X | X | X | X | X |

Bispecific Antibody

Bispecific antibody molecule as employed herein refers to a molecule with two antigen binding domains, which may bind different antigens. Examples of bispecific antibody formats include but are not limited to Bispecific T cell engager (BITE), F(ab′)2, F(ab′)-ScFv2, di-scFv, diabody, minibody, scFv-Fc, DART, TandAb, ScDiabody, ScDiabody-CH3, Diabody-CH3, triple body, miniantibody, minibody, TriBi minibody, ScFv-CH3 KIH (knobs in holes), Fab-ScFv, SCFv-CH-CL-scFv, scFv-KIH, Fab-scFv-Fc, Tetravalent HCAb, scDiabody-Fc, Diabody-Fc, intrabody, dock and lock antibodies, ImmTAC, HSAbody, ScDiabody-HAS, humabody and Tandem ScFv-toxic (see for example Christoph Spiess et al, Molecular Immunology 67 (2015) page 95-106).

The at least two binding domains and the variable domains (VH/VL) of the multispecific antibody may comprise peptide linkers (spacer peptides). The term “peptide linker” comprises in accordance with the present disclosure an amino acid sequence by which the amino acid sequences of one (variable and/or binding) domain and another (variable and/or binding) domain of the antibody construct of the present disclosure are linked with each other. The peptide linkers can also be used to fuse the third domain to the other domains of the antibody construct of the present disclosure. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antibody construct of the present disclosure. Illustrative bispecific single chain antibody constructs are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56.

Bivalent (also called divalent) or bispecific single-chain variable fragments (bi-scFvs or di-scFvs having the format (scFv)2 can be engineered by linking two scFv molecules (e.g. with linkers as described hereinbefore). If these two scFv molecules have the same binding specificity, the resulting (scFv)2 molecule will preferably be called bivalent (i.e. it has two valences for the same target epitope). If the two scFv molecules have different binding specificities, the resulting (scFv)2 molecule will preferably be called bispecific. The linking can be done by producing a single peptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8).

In line with this disclosure either the first, the second or the first and the second domain may comprise a single domain antibody, respectively the variable domain or at least the CDRs of a single domain antibody. Single domain antibodies comprise merely one (monomeric) antibody variable domain which is able to bind selectively to a specific antigen, independently of other V regions or domains. The first single domain antibodies were engineered from heavy chain antibodies found in camelids, and these are called VHH fragments. Cartilaginous fishes also have heavy chain antibodies (IgNAR) from which single domain antibodies called VNAR fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

Whether or not an antibody construct competes for binding with another given antibody construct can be measured in a competition assay such as a competitive ELISA or a cell-based competition assay. Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added and any additional binding is determined. Possible means for the read-out includes flow cytometry.

Bispecific T Cell Engager

A “BiTE” generally refers to a single polypeptide chain molecule that has two antigen binding domains, one of which binds to an immune effector cell antigen (e.g., CD3) and the second of which binds to an antigen present on the surface of a target cell.

In some embodiments, one of the antigen binding domain is specific for an immune cell, such as a T cell antigen, such as the CD3 receptor, expressed on the surface of T cells. In some embodiments, the second antigen binding domain binds to a tumor cell via a tumor-specific molecule. Accordingly, BiTEs are able to form a link between T cells and tumor cells by virtue of their specificities for an antigen on the T cell and an antigen on the tumor cell. This leads to activation of the T-cells and may trigger the T cells to exert their cytotoxic effects on tumor cells, independently of MHC I or co-stimulatory molecules. Examples of BITE based therapies currently approved or undergoing clinical trials include for example blinatumomab (Blyncyto®) which targets CD19 and is for the treatment of non-Hodgkin's lymphoma and acute lymphoblastic leukemia and solitomab which targets EpCAM and is for treating gastrointestinal and lung cancers.

In some embodiments, the bispecific antibody described in the present disclosure is a BiTE which is specific for at least a surface antigen on a T cell of interest. Examples of T cell surface antigens include but are not limited to: CD3, CD2, VLA-1, CD8, CD4, CCR6, CXCR5, CD25, CD31, CD45RO, CD197, CD127, CD38, CD27, CD196, CD277 and CXCR3, particularly CD2, CD3, CD31 and CD277.

BiTE molecules have been constructed to various target antigens including CD19, EpCAM, Her2/neu, EGFR, CD66e (or CEA, CEACAM5), CD33, EphA2, MCSP (or HMW-MAA), CD22, CD79a, CD79b, and sIgM. BiTE molecules are typically produced as recombinant, glycosylated proteins secreted by higher eukaryotic cell lines.

In some embodiments, the BiTE of the present disclosure is composed of a non-target cell antigen-binding fragment and a target immune cell-antigen binding fragment coupled together by a linker. Immune cells include, e.g., natural killer (NK) cells, T cells including cytotoxic T cells, or B cells, but also cells of the myeloid lineage can be regarded as immune cells, such as monocytes or macrophages, dendritic cells and neutrophilic granulocytes. Hence, said immune cell is in various embodiments, an NK cell, a T cell, a B cell, a monocyte, a macrophage, a dendritic cell or a neutrophilic granulocyte. As relevant here, the immune cell may be any target cell whose in vivo transduction is desired, the BiTE having the effect of increasing the transduction efficiency of the virus for that immune cell. In order to avoid non-specific interactions, one may select a bispecific antibody that recognizes antigens on immune effector cells that are at least over-expressed by these immune effector cells compared to other cells in the body. Such antigens may include, but are not limited to, CD3, CD16, CD25, CD28, CD64, CD89, NKG2D and NKp46. In some embodiments, the immune effector cell antigen is a T cell antigen. In some embodiments, the immune effector cell antigen is CD3. Accordingly, in some embodiments, the BiTE of the present disclosure is composed of an antigen binding fragment and an anti-CD3 antigen binding fragment coupled together by a linker.

First Antigen of Multispecific Antibody

In some embodiments, the first antigen binding domain of the multispecific antibody binds to an immune cell antigen. In some embodiments, the immune cell antigen is a T-cell antigen. In some embodiments, the T-cell antigen is selected from the group consisting of, CD3, CD4, CD8, and TCR.

In some embodiments, the first antigen of the multispecific antibody is CD3. The CD3 receptor complex is a protein complex and is composed of four chains. In mammals, the complex contains a CD3γ (gamma) chain, a CD3δ (delta) chain, and two CD3ε (epsilon) chains. These chains associate with the T cell receptor (TCR) and the so-called ζ (zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3ε gene which resides on chromosome 11. In some embodiments, the first antigen is CD3 epsilon. In some embodiments, the antigen epitope comprise amino acid residues 1-27 of the human CD3 epsilon extracellular domain.

In some embodiments, the first antigen of the multispecific antibody is CD4 (Cluster of Differentiation 4). CD4 is a transmembrane glycoprotein of the immunoglobulin superfamily, expressed on developing thymocytes, major histocompatibility class II (class II MHC)-restricted mature T lymphocytes and, in humans, on cells of the macrophage/monocyte lineage. On lymphoid cells, CD4 plays a critical role during thymocyte ontogeny and in the function of mature T cells. CD4 binds to non-polymorphic regions of class II MHC acting as a co-receptor for the T-cell antigen receptor (TCR). It increases avidity between thymocytes and antigen-presenting cells and contributes directly to signal transduction through association with the Src-like protein tyrosine kinase p56lck. CD4 is also a co-receptor for the human and simian immunodeficiency viruses (HIV-1, HIV-2, and SIV). Specifically, CD4 is a receptor for human immunodeficiency virus (HIV)-gp120 glycoprotein. Clinically, CD4 antibodies may be used to achieve immunological tolerance to grafts and transplants; treat autoimmune diseases and immune deficiency-related disorders such as, e.g., lupus, diabetes, rheumatoid arthritis, etc.; treat leukemias and lymphomas expressing CD4; as well as to treat HIV infection.

In some embodiments, the first antigen of the multispecific antibody is CD8 (Cluster of Differentiation 8). CD8 is a cell surface glycoprotein predominantly expressed on cytotoxic T lymphocytes, but also expressed on subsets of dendritic cells, natural killer cells, natural killer T cells, and γδT cells. The glycoprotein consists of two isoforms, α and β, which are encoded by different genes, and expressed as αα homodimers or αβ heterodimers, the latter of which is dominant. The CD8 co-receptors stabilize T cell receptor MHC-1 interaction and initiate intracellular signaling via lymphocyte-specific protein tyrosine kinase (Lck) phosphorylation of CD3-associated immunoreceptor tyrosine-based activation motifs (ITAMs) for activation. The amino acid sequence of full-length human CD8α is provided in UniProt as accession number P01732. The amino acid sequence of human full-length CD8β is provided in UniProt as accession number P10966. The term “CD8” includes full length CD8α or CD8β, recombinant CD8, fragments thereof, and fusions thereof. The term also encompasses CD8α or CD8β, or a fragment thereof, coupled to, for example, histidine tag, mouse or human Fc, or a signal sequence.

In some embodiments, the first antigen of the multispecific antibody is CTLA-4 (cytotoxic T-lymphocyte-associated protein 4). CTLA-4, also known as CD152, is a single pass type I membrane protein that forms a disulfide linked homo-dimer. Alternate splice variants, encoding different isoforms, have been characterized including a soluble isoform which functions as a monomer. CTLA-4's surface expression is tightly regulated by restricted trafficking to the cell surface and rapid internalization. The extracellular region of CTLA-4 comprises a single extracellular Ig(V) domain, followed by a transmembrane (TM) region and a small intracellular cytoplasmic tail (about 37 amino acids). The intracellular tail contains two tyrosine-based motifs, which interact with several intracellular proteins, including the lipid kinase phosphatidylinositol 3-kinase (PI3K), the phosphatases SHP-2 and PP2A and clathrin adaptor proteins AP-1 and AP-2. CTLA4 is homologous to CD28 and, similarly to CD28, binds to ligands CD80 (B7-1) and CD86 (B7-2). However, unlike CD28, binding of CTLA4 to B7 does not produce a stimulatory signal, but prevents the co-stimulatory signal normally provided by CD28. When a naïve T effector cell is activated through its T-cell receptors (TCRs), CTLA-4 is recruited to the cell surface and competes with CD28 (constitutively expressed on T-cells) for CD80/CD86, thereby shutting off further signaling through the TCR and thus down-regulating any further T-cell response by TCR signaling. Thus, CTLA-4 acts as a negative regulator of T effector cell activation that diminishes effector function and dictates the efficacy and duration of a T-cell response. In addition, CTLA-4 may play a role in enhancing the negative effect of regulatory T-cells on the immune response to cancer. CTLA-4 has a much higher affinity for members of the B7 family than for CD28, and therefore its expression on a T-cell dictates a dominant negative regulation of the T-cell. Blockage of CTLA-4 is reported to enhance T-cell responses.

In some embodiments, the first antigen of the multispecific antibody is a T cell receptor (TCR). TCR is a complex of membrane proteins that participate in the activation of T cells in response to the presentation of antigen. The TCR is responsible for recognizing antigens bound to major histocompatibility complex (MHC) molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. In some embodiments, the TCR may be modified on any cell comprising a TCR, including, for example, a helper T cell, a cytotoxic T cell, a memory T cell, regulatory T cell, natural killer T cell, and gamma delta T cell. Engagement of the TCR with antigen and MHC results in activation of its T lymphocyte through a series of biochemical events mediated by associated enzymes, co-receptors, and specialized accessory molecules. Each chain of the TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end. The variable domain of both the TCR α-chain and β-chain have three hypervariable or complementarity determining regions (CDRs). The constant domain of the TCR domain consists of short connecting sequences in which a cysteine residue forms a disulfide bond, making a link between the two chains. The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3- and ζ-chains, together with the TCR, form what is known as the T cell receptor complex. The signal from the T cell complex is enhanced by simultaneous binding of the MHC molecules by a specific co-receptor. On helper T cells, this co-receptor is CD4 (specific for class II MHC); whereas on cytotoxic T cells, this co-receptor is CD8 (specific for class I MHC). The co-receptor not only ensures the specificity of the TCR for an antigen, but also allows prolonged engagement between the antigen presenting cell and the T cell and recruits essential molecules (e.g., LCK) inside the cell involved in the signaling of the activated T lymphocyte. The term “T-cell receptor” is thus used in the conventional sense to mean a molecule capable of recognizing a peptide when presented by an MHC molecule.

Second Antigen of Multispecific Antibody

In some embodiments, the second antigen binding domain of the multispecific antibody binds to an antigen present on the surface of a cell targeted by the immune effector cell (e.g., a tumor cell). In some embodiments, the second antigen is selected from the group consisting of CD19, EpCAM, CD20, CD123, BCMA, B7-H3, and PSMA. In some embodiments, the second antigen is selected from the group consisting of CD19, EpCAM, Her2/neu, EGFR, CD66e, CD33, EphA2, MCSP, CD22, CD79a, CD79b, and sIgM. In some embodiments, the second antigen is CD19.

In some embodiments, the second antigen of the multispecific antibody is EpCAM (Epithelial cell adhesion molecule). EpCAM, also named as CD326 or “tumor-associated calcium signal transducer 1” refers to a 40 kDa type I transmembrane glycoprotein that consists of two epidermal growth factor-like extracellular domains, a cysteine-poor region, a transmembrane domain, and a short cytoplasmic tail. Human EpCAM is encoded by the GA733-2 gene on the long arm of chromosome 4 and is involved in cell to cell adhesion. EpCAM is expressed on the majority of epithelial tissues. Sequences of EpCAM are well known in the art. Human EpCAM is a human cell surface glycoprotein antigen associated with carcinomas of various origins, including colorectal, pancreatic, head, neck, ovarian, lung, cervical, prostate, and breast carcinomas. Malignant cell proliferation is often always associated with EpCAM expression at some stage of tumor development and high levels of EpCAM expression negatively correlate with cell differentiation. High levels of EpCAM expression have been shown to correlate with poor survival among breast cancer patients.

In some embodiments, the second antigen of the multispecific antibody is CD19 (Cluster of Differentiation 19). CD19 is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. CD19 is expressed on most B lineage cancers, including, e.g., acute lymphoblastic leukaemia, chronic lymphocyte leukaemia and non-Hodgkin's lymphoma. It is also an early marker of B cell progenitors. In some embodiments, the CD19 protein is expressed on a cancer cell. In some embodiments, “CD19” includes proteins comprising mutations, e.g., point mutations, fragments, insertions, deletions and splice variants of full length wild-type protein.

In some embodiments, the second antigen of the multispecific antibody is CD20. CD20 is also known as B lymphocyte CD20 antigen, MS4A1, B lymphocyte surface antigen B1, Bp35, or Leukocyte surface antigen Leu-16. The term CD20 includes human CD20 (AH003353; GenBank Accession nos. M27395, J03574). The major form of human CD20 comprises a 297 amino acid protein described by GenBank Protein ID 23110989. Examples of CD20 sequences include, but are not limited to NCBI reference numbers NP_068769.2 and NP_690605.1. CD20 expression are found on lymphomas (e.g. B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma) c) marginal zone lymphomas (including extranodal marginal zone B cell lymphoma (Mucosa-associated lymphatic tissue lymphomas, MALT), nodal marginal zone B cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma) f) hairy cell leukemia, g) lymphocytic lymphoma, waldenstrom's macroglobulinemia, h) acute lymphocytic leukemia (ALL), chronic lymphocytic leukemia (CLL)/small lymphocytic lymphoma (SLL), B-cell prolymphocytic leukemia, i) plasma cell neoplasms, plasma cell myeloma, multiple myeloma, plasmacytoma j) Hodgkin's disease. In some embodiments, the CD20 expressing cancer is a B-Cell Non-Hodgkin's lymphomas (NHL). In some embodiments, the CD20 expressing cancer is a Mantle cell lymphoma (MCL), acute lymphocytic leukemia (ALL), chronic lymphocytic leukemia (CLL), B-cell diffuse large cell lymphoma (DLCL), Burkitt's lymphoma, hairy cell leukemia, follicular lymphoma, multiple myeloma, marginal zone lymphoma, post transplant lymphoproliferative disorder (PTLD), HIV associated lymphoma, waldenstrom's macroglobulinemia, or primary CNS lymphoma. In some embodiments, “CD20” includes proteins comprising mutations, e.g., point mutations, fragments, insertions, deletions and splice variants of full length wild-type protein.

In some embodiments, the second antigen of the multispecific antibody is CD123. CD123 is also known as Cluster of Differentiation 123, Interleukin-3 receptor alpha chain, and IL3RA. CD123 is a type I transmembrane glycoprotein, with an extracellular domain comprising a predicted Ig-like domain and two FnIII domains. The term “CD123” may refer to any isoform of CD123. CD123 is preferentially expressed on certain types of pluripotent stem cells and cancer cells, such as leukemia cancer cells (e.g., acute myeloid leukemia cells). In some embodiments, “CD123” includes proteins comprising mutations, e.g., point mutations, fragments, insertions, deletions and splice variants of full length wild-type protein.

In some embodiments, the second antigen of the multispecific antibody is BCMA. The term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells, and plasma cells. Its ligand is called B-cell activator of the TNF family (BAFF) and a proliferation inducing ligand (APRIL). BCMA is involved in mediating the survival of plasma cells for maintaining long-term humoral immunity. The gene for BCMA is encoded on chromosome 16 producing a primary mRNA transcript of 994 nucleotides in length (NCBI accession NM_001192.2) that encodes a protein of 184 amino acids (NP_001183.2). Additional transcript variants have been described with unknown significance (Smirnova A S et al. Mol Immunol., 2008, 45(4):1179-1183. A second isoform, also known as TV4, has been identified (Uniprot identifier Q02223-2). As used herein, “BCMA” includes proteins comprising mutations, e.g., point mutations, fragments, insertions, deletions and splice variants of full length wild-type BCMA. In some embodiments, BCMA is expressed on the cell surface of malignant B-cells of a patient. In some embodiments, “BCMA” includes proteins comprising mutations, e.g., point mutations, fragments, insertions, deletions and splice variants of full length wild-type protein.

In some embodiments, the second antigen of the multispecific antibody is PSMA. PSMA is also known as prostate-specific membrane antigen or folate hydrolase 1 (FOLH1). The amino acid sequence of human PSMA can be found as UniProt/Swiss-Prot Accession No. Q04609 and the NCBI Reference Sequence ID number for the amino acid sequence of human PSMA is NP_004467.1. PSMA is an integral, non-shed membrane glycoprotein that is highly expressed in prostate epithelial cells and is a cell-surface marker for prostate cancer. In some embodiments, “PSMA” includes proteins comprising mutations, e.g., point mutations, fragments, insertions, deletions and splice variants of full length wild-type protein.

In some embodiments, the second antigen of the multispecific antibody is HER2/Neu. HER2/neu is a 185 kDa receptor protein that was originally identified as the product of the ERBB2 transforming gene from neuroblastomas of chemically treated rats. HER2/neu has been extensively investigated because of its role in several human carcinomas and in mammalian development. The sequence of human HER2/neu is available in GenBank, as accession number X03363. HER2/neu comprises four domains: an extracellular domain to which ligand binds; a lipophilic transmembrane domain; a conserved intracellular tyrosine kinase domain; and a carboxyl-terminal signaling domain harboring several tyrosine residues that can be phosphorylated. The sequence of the HER2/neu extracellular domain (ECD) is available in Protein DataBank Record 1S78 (2004). HER2/neu functions as a growth factor receptor and is often expressed by cancer cells of breast cancer, ovarian cancer or lung cancer. HER2/neu is overexpressed in 25-30% of human breast and ovarian cancers, and its overexpression is associated with aggressive clinical progression and poor prognosis in affected. In some embodiments, “Her2/Neu” includes proteins comprising mutations, e.g., point mutations, fragments, insertions, deletions and splice variants of full length wild-type protein.

In some embodiments, the second antigen of the multispecific antibody is EGFR. EGFR, also known as human epidermal growth factor receptor, HER-1 or ErbB1, is a 170 kDa transmembrane receptor encoded by the c-erbB proto-oncogene, and exhibits intrinsic tyrosine kinase activity. SwissProt database entry P00533 provides the sequence of EGFR. There are also isoforms and variants of EGFR (e.g., alternative RNA transcripts, truncated versions, polymorphisms, etc.) including but not limited to those identified by Swissprot database entry numbers P00533-1, P00533-2, P00533-3, and P00533-4. EGFR is known to bind ligands including α), epidermal growth factor (EGF), transforming growth factor-α (TGf-amphiregulin, heparin-binding EGF (hb-EGF), betacellulin, and epiregulin. EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis. In some embodiments, “EGFR” includes proteins comprising mutations, e.g., point mutations, fragments, insertions, deletions and splice variants of full length wild-type protein.

In some embodiments, the second antigen of the multispecific antibody is CD33 (Cluster of Differentiation 33). CD33 is an antigenic determinant detectable on leukemia cells as well on normal precursor cells of the myeloid lineage. The human and murine amino acid and nucleic acid sequences can be found in a public database, such as GenBank, UniProt and Swiss-Prot. For example, the amino acid sequence of human CD33 can be found as UniProt/Swiss-Prot Accession No. P20138 and the nucleotide sequence encoding of the human CD33 can be found at Accession No. NM_001772.3. In some embodiments, the CD33 protein is expressed on a cancer cell. Certain hematological malignancies are characterized by the expression of CD33 on the surface of the malignant cells. CD33-positive hematological malignancies include, but are not limited to, acute myeloid leukemia (AML), chronic myeloid leukemia (CML), chronic myelomonocytic leukemia, thrombolytic leukemia, a myelodysplastic syndrome, a myeloproliferative disorder, refractory anemia, a preleukemia syndrome, a lymphoid leukemia, or an undifferentiated leukemia. In some embodiments, “CD33” includes proteins comprising mutations, e.g., point mutations, fragments, insertions, deletions and splice variants of full length wild-type protein.

In some embodiments, the second antigen of the multispecific antibody is EphA2 (Ephrin receptor A2). EphA2 is found to be either overexpressed, mutated or amplified in various cancers. The nucleotide and/or amino acid sequences of EphA2 polypeptides can be found in the literature or public databases, or the nucleotide and/or amino acid sequences can be determined using cloning and sequencing techniques known to one of skill in the art. For example, the nucleotide sequence of human EphA2 can be found in the GenBank database (see, e.g., Accession Nos. BC037166, M59371 and M36395). The amino acid sequence of human EphA2 can be found in the GenBank database (see, e.g., Accession Nos. AAH37166 and AAA53375). In some embodiments, “EphA2” includes proteins comprising mutations, e.g., point mutations, fragments, insertions, deletions and splice variants of full length wild-type protein.

In some embodiments, the second antigen of the multispecific antibody is MCSP (Melanoma Chondroitin Sulfate Proteoglycan). MCSP is also known as chondroitin sulfate proteoglycan 4 (CSPG4), chondroitin sulfate proteoglycan NG2, high molecular weight-melanoma associated antigen (HMW-MAA), and melanoma chondroitin sulfate proteoglycan. The amino acid sequence of an exemplary human MCSP is shown in Genbank Accession No: NP_001888. It is an early cell surface melanoma progression marker implicated in stimulating tumor cell proliferation, migration, and invasion. In some embodiments, “MCSP” includes proteins comprising mutations, e.g., point mutations, fragments, insertions, deletions and splice variants of full length wild-type protein.

In some embodiments, the second antigen of the multispecific antibody is CD66e, which is also known as carcinoembryonic antigen (CEA), carcinoembryonic antigen-related cell adhesion molecule 5 (CEACAM5), or CD66. As immunoglobulin (Ig) family members, it is a glycosyl phosphatidyl inositol (GPI)-cell surface anchored glycoprotein that possesses six Ig C2-type domains. Expression of CD66e can be found in a large number of tumors of epithelial origin. The nucleotide and amino acid sequences encoding CEA are known in the art and are easily retrievable by known methods. The amino acid sequence of human CEA is depicted in GenBank accession number NM_004363. In some embodiments, “CD66e)” includes proteins comprising mutations, e.g., point mutations, fragments, insertions, deletions and splice variants of full length wild-type protein.

In some embodiments, the second antigen of the multispecific antibody is B7-H3 (also known as CD276). It is a member of the B7 family of immune cell modulating molecules having a single-pass transmembrane. In humans the B7-H3 protein is expressed in two forms: Variant 1 contains a V-like or C-like Ig domain at two sites, respectively, and Variant 2 contains a V-like or C-like Ig domain at one site, respectively. The C-terminal intracellular domain of B7-H3 contains 45 amino acids. It is expressed on the surface of a wide variety of tumor cells and tumor vasculature including neuroblastoma, melanoma, renal cell cancer, prostate cancer, colorectal cancer, pancreatic cancer, gastric cancer, breast cancer, ovarian cancer and small cell lung cancer. B7-H3 expression has been correlated with poor prognosis in ovarian, RCC, NSCLC, pancreatic cancer, prostate cancer and colon cancer, with a potential role for inhibition of cytotoxic lymphocyte activity. In some embodiments, “B7-H3” includes proteins comprising mutations, e.g., point mutations, fragments, insertions, deletions and splice variants of full length wild-type protein.

In some embodiments, the second antigen of the multispecific antibody is CD22. CD22, also known as SIGLEC-2 (UniProt P20273), is a cell-surface receptor that is expressed on mature B-cells. CD22 contains multiple Ig domains and is a member of the immunoglobulin superfamily. The extracellular domain of CD22 interacts with sialic acid moieties, including those present on the CD45 cell surface protein. CD22 is thought to function as an inhibitory receptor for B-cell receptor signaling. CD22 is expressed on the surface of many types of malignant B cells, including but not limited to, acute lymphocytic leukemia (B-ALL), chronic B-lymphocytic cells (B-CLL), B lymphoma cells such as Burkitt's, AIDS-associated and follicular lymphomas, and hairy cell leukemias, as well as on normal mature B lymphocytes. Along with CD20 and CD 19, the restricted B-cell expression of CD22 makes it a target for the therapeutic treatment of B-cell malignancies. An example of CD22 specific antibody is epratuzumab. In some embodiments, “CD22” includes proteins comprising mutations, e.g., point mutations, fragments, insertions, deletions and splice variants of full length wild-type protein.

In some embodiments, the second antigen of the multispecific antibody is CD79a. CD79a is an antigenic determinant known to be detectable on some malignant hematological cancer cells, e.g., leukemia cells. The human and murine amino acid and nucleic acid sequences can be found in a public database, such as GenBank, UniProt and Swiss-Prot. For example, the amino acid sequences of human CD79a can be found at Accession Nos. NP_001774.1 (isoform 1 precursor) or NP_067612.1 (isoform 2 precursor), and the mRNA sequences encoding them can be found at Accession Nos. NM_001783.3 (transcript variant 1) or NM_021601.3 (transcript variant 2). In some embodiments, the CD79a protein is expressed on a cancer cell. In some embodiments, the multispecific antibody binds an antigen within the extracellular domain of the CD79a protein. In some embodiments, “CD79a” includes proteins comprising mutations, e.g., point mutations, fragments, insertions, deletions and splice variants of full length wild-type protein.

In some embodiments, the second antigen of the multispecific antibody is CD79b. CD79b is an antigenic determinant known to be detectable on some malignant hematological cancer cells, e.g., leukemia cells. The human and murine amino acid and nucleic acid sequences can be found in a public database, such as GenBank, UniProt and Swiss-Prot. For example, the amino acid sequences of human CD79b can be found at Accession Nos. NP_000617.1 (isoform 1 precursor), NP_067613.1 (isoform 2 precursor), or NP_001035022.1 (isoform 3 precursor), and the mRNA sequences encoding them can be found at Accession Nos. NM_000626.2 (transcript variant 1), NM_021602.2 (transcript variant 2), or NM_001039933.1 (transcript variant 3). In some embodiments, the CD79b protein is expressed on a cancer cell. In some embodiments, the multispecific antibody binds an antigen within the extracellular domain of the CD79b protein. In some embodiments, “CD79b” includes proteins comprising mutations, e.g., point mutations, fragments, insertions, deletions and splice variants of full length wild-type protein.

In some embodiments, the second antigen of the multispecific antibody is sIgM. sIgM (surface immunoglobulin M) is typically expressed on B cells. In some embodiments, sIgM is expressed on a cancer cell. In some embodiments, the multispecific antibody binds an antigen within the extracellular domain of the sIgM. In some embodiments, “sIgM” includes proteins comprising mutations, e.g., point mutations, fragments, insertions, deletions and splice variants of full length wild-type protein.

In some embodiments, the second antigen of the multispecific antibody is DC-SIGN. DC-SIGN (Dendritic Cell-Specific Intercellular adhesion molecule-3-Grabbing Non-integrin) is also known as CD209 (Cluster of Differentiation 209), which is a protein encoded by the CD209 gene in human. DC-SIGN is a C-type lectin receptor present on the surface of both macrophages and dendritic cells. DC-SIGN on macrophages recognizes and binds with high affinity to high-mannose type N-glycans, a class of pathogen associated molecular patterns PAMPs commonly found on viruses, bacteria and fungi. This binding interaction activates phagocytosis. On myeloid and dendritic cells DC-SIGN mediates dendritic cell rolling interactions with blood endothelium and activation of CD4+ T cells, as well as recognition of pathogen haptens. In some embodiments, “DC-SIGN” includes proteins comprising mutations, e.g., point mutations, fragments, insertions, deletions and splice variants of full length wild-type protein.

In some embodiments, the second antigen of the multispecific antibody is CD11b. CD11b (ITGAM; integrin αM) is capable of forming heterodimer with CD18. It functions as a receptor for complement (C3bi), fibrinogen, or clotting factor X. In humans, CD11b is strongly expressed on myeloid cells and weakly expressed on NK cells and some activated lymphocytes as well as on microglia in the brain. In some embodiments, CD11b is also expressed on cancer cells and targeting it leads to anti-cancer effects. Exemplary relevant sequences for CD11 b can be found at Accession Nos. NP_001139280.1, NP_000623.2, XP_011544153.1, XP_011544152.1, XP_006721108.1, AAH99660.1, and AH004143.2. In some embodiments, “CD11b” includes proteins comprising mutations, e.g., point mutations, fragments, insertions, deletions and splice variants of full length wild-type protein.

In some embodiments, the second antigen of the multispecific antibody is CD11c. CD11c is also known as CD11C, CD11c antigen, Integrin alpha X, complement component 3 receptor 4 subunit, ITGAX, LeuM5, Integrin alpha X precursor, Leukocyte adhesion glycoprotein p150, p95 alpha chain, and Leukocyte adhesion receptor p150 subunit. Full length CD11c protein has amino acid sequence as set forth at Genbank Accession No. NP_000878 and encoded by a full-length nucleotide sequence as set forth at Genbank Accession No. NM_000887. In some embodiments, “CD11c” includes proteins comprising mutations, e.g., point mutations, fragments, insertions, deletions and splice variants of full length wild-type protein.

In some embodiments, the second antigen of the multispecific antibody is CD18. CD18 is also known as Integrin beta chain-2 or integrin 02, which in human is encoded by the ITGB2 gene. CD18 is capable of forming heterodimer with CD1 lb. Exemplary sequences for CD18 can be found at GenBank Accession No. NP_000202 (amino acid sequence) and GenBank Accession No. NM_000211 (nucleic acid). In some embodiments, “CD18” includes proteins comprising mutations, e.g., point mutations, fragments, insertions, deletions and splice variants of full length wild-type protein.

Antibody Subtype

In some embodiments, the multispecific antibody as described herein is an IgG1, an IgG2, an IgG3 or an IgG4 isotype.

In some embodiments, the multispecific antibody is the IgG1 isotype. In some embodiments, the multispecific antibody is the IgG2 isotype. In some embodiments, the multispecific antibody is the IgG3 isotype. In some embodiments, the multispecific antibody is the IgG4 isotype.

In some embodiments, the multispecific antibody comprises one or more Fc substitutions that reduces binding of the multispecific antibody to a Fcγ receptor (FcγR).

In some embodiments, the one or more Fc substitutions is selected from the group consisting of F234A/L235A on IgG4, L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4 and S228P/F234A/L235A/G236-deleted/G237A/P238S on IgG4, wherein residue numbering is according to the EU index.

In some embodiments, the multispecific antibody further comprises a S228P substitution.

In some embodiments, the multispecific antibody comprises one or more asymmetric substitutions in a first CH3 domain or in a second CH3 domain, or in both the first CH3 domain and the second CH3 domain.

In some embodiments, one or more asymmetric substitutions is selected from the group consisting of F450L/K409R, wild-type/F409L_R409K, T366Y/F405A, T366W/F405W, F405W/Y407A, T394W/Y407T, T394S/Y407A, T366W/T394S, F405W/T394S and T366W/T366SL368AY407V, L351YF405AY407V/T394W, T366I_K392MT394W/F405AY407V, T366LK392MT394W/F405AY407V,

L351YY407A/T366AK409F, L351YY407A/T366VK409F, Y407A/T366AK409F and T350V_L351Y_F405AY407V/T350V_T366L_K392L_T394W.

Methods of Generating Antibodies

The antibodies used in the methods of the present disclosure binding specific antigens may be selected de novo from, for example, a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-14, 1996; Sheets et al., PITAS (USA) 95:6157-62, 1998; Hoogenboom and Winter, J Mol Biol 227:381, 1991; Marks et al., J Mol Biol 222:581, 1991). Phage display libraries expressing antibody heavy and light chain variable regions as fusion proteins with bacteriophage pIX coat protein as described in Shi et al (2010) J. Mol. Biol. 397:385-96 and Int'l Pat. Pub. No. WO2009/085462. The antibody libraries may be screened for binding to the desired antigen, such as BCMA, CD3, CD38, CD123, CD19, CD33, PSMA or TMEFF2 extracellular domain and the obtained positive clones may be further characterized and the Fabs isolated from the clone lysates, and subsequently cloned as full length antibodies. Such phage display methods for isolating human antibodies are established in the art. See for example: U.S. Pat. Nos. 5,223,409; 5,403,484; 5,571,698; 5,427,908; 5,580,717; 5,969,108; 6,172,197; 5,885,793; 6,521,404; 6,544,731; 6,555,313; 6,582,915; and 6,593,081.

Multispecific antibody (e.g., bispecific antibody) may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. In some embodiments, a reducing agent is selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Exemplary CH3 mutations that may be used in a first heavy chain and in a second heavy chain of the bispecific antibody are K409R and/or F405L.

Additional CH3 mutations that may be used include technologies such as Duobody® mutations (Genmab), Knob-in-Hole mutations (Genentech), electrostatically-matched mutations (Chugai, Amgen, NovoNordisk, Oncomed), the Strand Exchange Engineered Domain body (SEEDbody) (EMD Serono), and other asymmetric mutations (e.g. Zymeworks).

Duobody® mutations (Genmab) are disclosed for example in U.S. Pat. No. 9,150,663 and US2014/0303356 and include mutations F405L/K409R, wild-type/F405L_R409K, T350I_K370TF405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351YF405AY407V/T394W, T366IK392MT394W/F405AY407V, T366LK392MT394W/F405AY407V, L351YY407A/T366AK409F, L351YY407A/T366VK409F, Y407A/T366AK409F, or T350V_L351YF405AY407V/T350V_T366LK392LT394W as described in US2012/0149876 or US2013/0195849.

SEEDbody mutations involve substituting select IgG residues with IgA residues to promote heavy chain heterodimerization as described in US20070287170.

Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351YY407A/T366AK409F, L351YY407A/T366VK409F, K392D/D399K, K392D/E356K, K253ED282KK322D/D239KE240KK292D, K392D_K409D/D356K_D399K as described in WO2007/147901, WO 2011/143545, WO2013157954, WO2013096291 and US2018/0118849.

Additional bispecific or multispecific structures that can be used as T cell redirecting therapeutics include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine-China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Fc Engineering of Antibodies

The Fc region of the T cell redirecting therapeutics such as bispecific or multispecific antibodies may comprise at least one substitution in the Fc region that reduces binding of the T cell redirecting therapeutics to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4.

Fc substitutions that may be used to reduce CDC is a K322A substitution.

Well-known S228P substitution may further be made in IgG4 antibodies to enhance IgG4 stability.

An exemplary wild-type IgG1 comprises an amino acid sequence of SEQ ID NO: 31, as below:

(SEQ ID NO: 31)
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVF
SCSVMHEALHNHYTQKSLSLSPGK

An exemplary wild-type IgG4 comprises an amino acid sequence of SEQ ID NO: 32, as below:

(SEQ ID NO: 32)
ASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKY
GPPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK

In Vivo Delivery of Polynucleotides Encoding a Multispecific Antibody

In some embodiments, instead of a multispecific antibody, a polynucleotide encoding the multispecific antibody is administered to the subject which allows the production of the multispecific antibody in vivo. In some embodiments, the administration of such polynucleotide generates similar effect in vivo as direct administration of multispecific antibody. In some embodiments, the administration of such polynucleotide improves the in vivo transduction efficiency of a vector. In some embodiments, the polynucleotide is an mRNA.

In some embodiments, in vivo delivery of such polynucleotides generates multispecific antibody expression over time (e.g., starting within hours and lasting several days). In some embodiments, in vivo delivery of such polypeptides results in desirable pharmacokinetics, pharmacodynamics and/or safety profile of the encoded multispecific antibody. In some embodiments, the polynucleotide may be optimized by one or more means to prevent immune activation, increase stability, reduce any tendency to aggregate, such as over time, and/or to avoid impurities. Such optimization may include the use of modified nucleosides, modified, and/or particular 5′ UTRs, 3′UTRs, and/or poly(A) tail modifications for improved intracellular stability and translational efficiency (see, e.g., Stadler et al., 2017, Nat. Med.). Such modifications are known in the art.

Strategies for in vivo delivery of polynucleotide (e.g., mRNA) are known in the art. For a summary of strategies, see Mol Ther. 2019 Apr. 10; 27(4): 710-728, which is incorporated herein by reference in its entirety.

In some embodiments, the polynucleotide encoding a multispecific antibody is co-formulated into lipid nanoparticles (LNPs). In some embodiments, LNP formulations are composed of (1) an ionizable or cationic lipid or polymeric material, bearing tertiary or quaternary amines to encapsulate the polyanionic mRNA; (2) a zwitterionic lipid (e.g., 1,2-dioleoyl-sn-glycero-3-phosphoethanolamine [DOPE]) that resembles the lipids in the cell membrane; (3) cholesterol to stabilize the lipid bilayer of the LNP; and (4) a polyethylene glycol (PEG)-lipid to lend the nanoparticle a hydrating layer, improve colloidal stability, and reduce protein absorption.

In some embodiments, the polynucleotide encoding a multispecific antibody is delivered via formulation with cationic or ionizable lipids and lipid-like agents. In some embodiments, the cationic lipids bear alkylated quaternary ammonium groups and retain their cationic nature in a pH-independent fashion. In some embodiments, the lipids are ionizable (e.g., acquire positive charges by protonation of free amines as pH is lowered). In some embodiments, the cationic lipids comprise N-[1-(2,3-dioleoyloxy)propyl]-N,N,N-trimethylammonium chloride (DOTMA). In some embodiments, the ionizable lipids comprise Dlin-MC3-DMA (MC3).

In some embodiments, the polynucleotide encoding a multispecific antibody is delivered via formulation with polymeric materials. In some embodiments, the polymeric material comprise low-molecular-weight polyethyleneimine (PEI) modified with fatty chains. In some embodiments, the polymeric material comprises poly(glycoamidoamine) polymers modified with fatty chains. In some embodiments, the polymeric material comprises Poly(O-amino)esters (PBAEs)

In some embodiments, the polynucleotide encoding a multispecific antibody is delivered via formulation with dendrimers (e.g., polyamidoamine (PAMAM) or polypropylenimine-based dendrimers) or Cell-Penetrating Peptides (CPPs). In some embodiments, the polynucleotide is covalently linked with CPP.

Examples of Multispecific Antibodies

In some embodiments, the multispecific antibody is a BCMA×CD3 bispecific antibody, a GPRC5D×CD3 bispecific antibody, a CD33×CD3 bispecific antibody, a CD19×CD3 bispecific antibody, a CD123×CD3 bispecific antibody, a PSMA×CD3 bispecific antibody, or a TMEFF2×CD3 bispecific antibody.

In some embodiments, the multispecific antibody is the BCMA×CD3 bispecific antibody. In some embodiments, the multispecific antibody is the GPRC5D×CD3 bispecific antibody. In some embodiments, the multispecific antibody is the CD33×CD3 bispecific antibody. In some embodiments, the multispecific antibody is the CD19×CD3 bispecific antibody. In some embodiments, the multispecific antibody is the CD123×CD3 bispecific antibody. In some embodiments, the multispecific antibody is the PSMA×CD3 bispecific antibody. In some embodiments, the multispecific antibody is the TMEFF2×CD3 bispecific antibody.

In some embodiments, the multispecific antibody binds CD3 epsilon (CDR), CD8, KI2L4, NKG2E, NKG2D, NKG2F, BTNL3, CD186, BTNL8, PD-1, CD195, or NKG2C.

In some embodiments, the multispecific antibody that binds CD19 comprises a CD19 binding domain of blinatumomab, axicabtagene ciloleucel, tisagenlecleucel-t, inebilizumab, lisocabtagene maraleucel, XmAb-5574, CIK-CAR.CD19, ICTCAR-011, IM-19, JCAR-014, loncastuximab tesirine, MB-CART2019.1, OXS-1550, PBCAR-0191, PCAR-019, PCAR-119, Sen1-001, TI-1007, XmAb-5871, PTG-01, PZO1, Sen1_1904A, Sen11904B, UCART-19, CSG-CD19, DI-B4, ET-190, GC-007F or GC-022.

In some embodiments, the multispecific antibody that binds CD19 comprises blinatumomab, axicabtagene ciloleucel, tisagenlecleucel-t, inebilizumab, lisocabtagene maraleucel, XmAb-5574, CIK-CAR.CD19, ICTCAR-011, IM-19, JCAR-014, loncastuximab tesirine, MB-CART2019.1, OXS-1550, PBCAR-0191, PCAR-019, PCAR-119, Sen1-001, TI-1007, XmAb-5871, PTG-01, PZ01, Sen1_1904A, Sen1_1904B, UCART-19, CSG-CD19, DI-B4, ET-190, GC-007F or GC-022.

In some embodiments, the multispecific antibody used in the present disclosure is blinatumomab. Blinatumomab is a CD19/CD3-bispecific antibody construct of the bispecific T cell engager (BiTE) class and comprises the amino acid sequence of SEQ ID NO: 34:

(SEQ ID NO: 34)
DIQLTQSPASLAVSLGQRATISCKASQSVDYDGDSYLNWYQQIPGQPPKLL
IYDASNLVSGIPPRFSGSGSGTDFTLNIHPVEKVDAATYHCQQSTEDPWTF
GGGTKLEIKGGGGSGGGGSGGGGSQVQLQQSGAELVRPGSSVKISCKASGY
AFSSYWMNWVKQRPGQGLEWIGQIWPGDGDTNYNGKFKGKATLTADESSST
AYMQLSSLASEDSAVYFCARRETTTVGRYYYAMDYWGQGTTVTVSSGGGGS
DIKLQQSGAELARPGASVKMSCKTSGYTFTRYTMHWVKQRPGQGLEWIGYI
NPSRGYTNYNOKFKDKATLTTDKSSSTAYMQLSSLTSEDSAVYYCARYYDD
HYCLDYWGQGTTLTVSSVEGGSGGSGGSGGSGGVDDIQLTQSPAIMSASPG
EKVTMTCRASSSVSYMNWYQQKSGTSPKRWIYDTSKVASGVPYRFSGSGSG
TSYSLTISSMEAEDAATYYCQQWSSNPLTFGAGTKLELKHHHHHH.

The CD19 binding region of Blinatumomab comprises the following CDRs:

CDRL1:
(SEQ ID NO: 35)
QSVDYDGDSY
CDRL2:
(SEQ ID NO: 36)
DAS
CDRL3:
(SEQ ID NO: 37)
QQSTEDPWT
CDRH1:
(SEQ ID NO: 38)
GYAFSSYW
CDRH2:
(SEQ ID NO: 39)
IWPGDGDT
CDRH3:
(SEQ ID NO: 40)
ARRETTTVGRYYYAMDY

The CD3 binding region of Blinatumomab comprises the following CDRs:

CDRH1:
(SEQ ID NO: 41)
GYTFTRYT
CDRH2:
(SEQ ID NO: 42)
INPSRGYT
CDRH3:
(SEQ ID NO: 43)
ARYYDDHYCLDY
CDRL1:
(SEQ ID NO: 44)
SSVSY
CDRL2:
(SEQ ID NO: 45)
DTS
CDRL3:
(SEQ ID NO: 46)
QQWSSNP

In some embodiments, the multispecific antibody is a CD19×CD3 bispecific antibody comprising a CD19 binding region comprising the CDRs according to SEQ ID NOS: 35-40 and/or a CD3 binding region comprising the CDRs according to SEQ ID NOS: 41-46. In some embodiments, the CD19×CD3 bispecific antibody is a CD19×CD3 BiTE.

### Methods

Various embodiments of the present disclosure provide methods of using a multispecific antibody as an enhancer of vector (e.g. non-viral vector or viral vector) transduction. In some embodiments, the multispecific antibody facilitate cell transduction of vectors, such as those designed for gene therapy and/or treatment of cancer or hematological malignancy in vivo. In some embodiments, both multispecific antibody and vector are administered in vivo. In various embodiments, the multispecific antibody may be administered before, concurrently with, or after the administration of viral vectors.

In some embodiments, the multispecific antibody and vector are administered to a subject to treat and/or prevent a disease, disorder, or condition. In some embodiments, the multispecific antibody and vector are administered to a subject for research and/or drug development purposes.

Effect

In some embodiments, administration of the multispecific antibody in a subject results in the activation of immune cells. In some embodiments, the activation of immune cells is mediated by the multispecific antibody's binding to both immune cells and cells expressing specific antigens.

In some embodiments, activation of immune cells is measured by the level of one or more cell markers. In some embodiments, activation of immune cells is measured by the percentage of the immune cells that are positive for one or more cell markers. In some embodiments, the immune cells are T cells (T lymphocytes) or NK cells. In some embodiments, the immune cells are CD4+ T cells or CD8+ T cells. In some embodiments, the one or more cell markers are selected from the groups consisting of CD71, CD25, CD69, Ki67, and any combination thereof.

In some embodiments, activation of immune cells is measured by the percentage of the immune cells that are CD71 positive. In some embodiments, administration of the multispecific antibody increases the percentage of the CD71+ immune cells by at least 10%, at least 20%, at least 30%, at least 40%, at least 50%, at least 60%, at least 70%, at least 80%, or at least 90%. In some embodiments, activation of immune cells is measured by the level of CD71 expressed on the surface of the immune cells. In some embodiments, administration of the multispecific antibody increases the level of CD71 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, activation of immune cells is measured by the percentage of the immune cells that are CD25 positive. In some embodiments, administration of the multispecific antibody increases the percentage of the CD25+ immune cells by at least 10%, at least 20%, at least 30%, at least 40%, at least 50%, at least 60%, at least 70%, at least 80%, or at least 90%. In some embodiments, activation of immune cells is measured by the level of CD25 expressed on the surface of the immune cells. In some embodiments, administration of the multispecific antibody increases the level of CD25 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, activation of immune cells is measured by the percentage of the immune cells that are CD69 positive. In some embodiments, administration of the multispecific antibody increases the percentage of the CD69+ immune cells by at least 10%, at least 20%, at least 30%, at least 40%, at least 50%, at least 60%, at least 70%, at least 80%, or at least 90%. In some embodiments, activation of immune cells is measured by the level of CD69 expressed on the surface of the immune cells. In some embodiments, administration of the multispecific antibody increases the level of CD69 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, activation of immune cells is measured by the percentage of the immune cells that are Ki67 positive. In some embodiments, administration of the multispecific antibody increases the percentage of the Ki67+ immune cells by at least 10%, at least 20%, at least 30%, at least 40%, at least 50%, at least 60%, at least 70%, at least 80%, or at least 90%. In some embodiments, activation of immune cells is measured by the level of Ki67 expressed on the surface of the immune cells. In some embodiments, administration of the multispecific antibody increases the level of Ki67 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, administration of the multispecific antibody in a subject results in an increase of immune cells that are susceptible and/or accessible to transduction of vector.

In some embodiments, administration of the multispecific antibody in a subject results in active proliferation of immune cells. In some embodiments, the proliferation of immune cells increase the number and/or susceptibility to transduction by vector.

In some embodiments, administration of the multispecific antibody in a subject results in a decrease of numbers of immune cells (e.g., T cells) in the GO phase and/or an increase of numbers of immune cells (e.g., T cells) in the non-GO phase.

In some embodiments, administration of the multispecific antibody in a subject increase the number and/or percentage of immune cells that are in a state of metabolic fitness for transduction of vector.

In some embodiments, administration of the multispecific antibody in a subject results in the accumulation of immune cells in lymph nodes. In some embodiments, administration of the multispecific antibody in a subject results in the accumulation of immune cells in tumor sites.

In some embodiments, administration of the multispecific antibody in a subject facilitates the entry of vector (e.g. virus particles) into target immune cells. In some embodiments, administration of the multispecific antibody in a subject enhance the infectious titers of vector particles. In some embodiments, administration of the multispecific antibody in a subject increase the cellular uptake of vector particles by immune cells.

In some embodiments, the multispecific antibody is a bispecific antibody. In some embodiments, the vector is a lentiviral vector. In some embodiments, the immune cells are T cells.

In some embodiments, the immune cells here are a subset of immune cells in vivo that can be recognized by at least one antigen-specific binding domain of the multispecific antibody. In some embodiments, the immune cells reside in the lymph nodes.

Administration Schedule

In some embodiments of the methods described herein, transduction (e.g., retroviral transduction, for example lentiviral transduction) of T lymphocytes (e.g., primary human T lymphocytes) can be enhanced upon administering to the subject the multispecific antibody before, concurrently with or after administering to the subject the vector, or any combination thereof for any of the periods of time disclosed herein.

In some embodiments, the multispecific antibody is administered before the vector is administered. In some embodiments, the multispecific antibody is administered about 0.5 hour, about 1 hour, about 2 hours, about 3 hours, about 4 hours, about 6 hours, about 9 hours, about 12 hours, about 16 hours, about 20 hours, about 1 day, about 2 days, about 3 days, about 4 days, about 5 days, about 6 days, or about 7 days, or longer, before the vector is administered. In some embodiments, when the multispecific antibody and/or vector are administered repeatedly, the time interval listed here is calculated based on the interval between the last administration of multispecific antibody and the first administration of vector.

In some embodiments, the multispecific antibody is administered after the vector is administered. In some embodiments, the multispecific antibody is administered about 0.5 hour, about 1 hour, about 2 hours, about 3 hours, about 4 hours, about 6 hours, about 9 hours, about 12 hours, or about 16 hours after the vector is administered. In some embodiments, when the multispecific antibody and/or vector are administered repeatedly, the time interval listed here is calculated based on the interval between the last administration of vector and the first administration of multispecific antibody.

In some embodiments, the multispecific antibody is administered concurrently with the vector. The term “concurrently” as used herein is not limited to the administration of therapeutic agents at exactly the same time, but rather it is meant that the multispecific antibody and the vector are administered to a subject and/or cells in a sequence and within a time interval such that they can act together on the target cells. For example, each agent may be administered sufficiently close in time so as to provide the desired therapeutic or prophylactic effect, e.g., within about 10 minutes, within about 20 minutes, within about 30 minutes, within about 60 minutes, within about 2 hours, within about 3 hours, within about 6 hours, within about 12 hours, or within about 24 hours. Each agent can be administered to a subject separately, in any appropriate form and by any suitable route. Each agent in a concurrent administration may be administered in the same medicament (simultaneously), in separate medicaments administered one after the other in any order, or sequentially in any order.

In some embodiments, when the multispecific antibody and/or the vector are administered repeatedly, at least one administration of the multispecific antibody occurs concurrently with at least one administration of the vector. In some embodiments, the first, or only, administration of the vector occurs concurrently with the last, or only, administration of the multispecific antibody. In some embodiments, the first, or only, administration of the multispecific antibody occurs concurrently with the last, or only, administration of the vector. In some embodiments, each administration of the multispecific antibody occurs concurrently with the administration of the vector. In some embodiments, each administration of the vector occurs concurrently with the administration of the multispecific antibody.

The present disclosure further contemplates that one or more additional agents that improve the transduction efficiency of vector may be used in combination with the multispecific antibody and the vector described herein. And the one or more additional agents may be administered before, concurrently with or after administering to the subject the multispecific antibody and/or the vector.

Dosage

Vector Dosage

A vector may be used to infect cells in vivo at an any effective dosage. In some embodiments, the vector is administered to a subject in vivo, by direct injection to the cell, tissue, organ or subject in need of therapy.

Viral vectors may also be delivered according to viral titer (TU/mL). The amount of lentivirus directly injected is determined by total TU and can vary based on both the volume that could be feasibly injected to the site and the type of tissue to be injected. In some embodiments, the viral titer delivered is about 1×10⁵ to 1×10⁶, about 1×10⁵ to 1×10⁷, 1×10⁵ to 1×10⁷, about 1×10⁶ to 1×10⁹, about 1×10⁷ to 1×10¹⁰, about 1×10⁷ to 1×10¹¹, or about 1×10⁹ to 1×10¹¹ or more per injection could be used. In some embodiments, the viral titer delivered is about 1×10⁶ to 1×10⁷, about 1×10⁶ to 1×10⁸, 1×10⁶ to 1×10⁹, about 1×10⁷ to 1×10¹⁰, about 1×10⁸ to 1×11¹¹, about 1×10⁸ to 1×10¹², or about 1×10¹⁰ to 1×10¹ or more per injection could be used. For example, a brain injection site may only allow for a very small volume of virus to be injected, so a high titer prep would be preferred, a TU of about 1×10⁶ to 1×10⁷, about 1×10⁶ to 1×10⁸, 1×10⁶ to 1×10⁹, about 1×10⁷ to 1×10¹⁰, about 1×10⁸ to 1×10¹¹, about 1×10⁸ to 1×10¹², or about 1×10¹⁰ to 1×10¹ or more per injection could be used. However, a systemic delivery could accommodate a much larger TU, a load of about 1×10⁸, about 1×10⁹, about 1×10¹⁰, about 1×10¹¹, about 1×10¹², about 1×10¹³, about 1×10⁴, or about 1×10¹⁵, could be delivered.

In some embodiments, the vector is administered at a dose of between about 1×10¹² and 5×10¹⁴ vector genomes (vg) of the vector per kilogram (vg) of total body mass of the subject (vg/kg). In some embodiments, the vector is administered at a dose of between about 1×10¹³ and 5×10¹⁴ vg/kg. In some embodiments, the vector is administered at a dose of between about 5×10¹³ and 3×10¹⁴ vg/kg. In some embodiments, the vector is administered at a dose of between about 5×10¹³ and 1×10¹⁴ vg/kg. In some embodiments, the vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the vector is administered at a dose of between about 1×10¹² and 5×10¹⁴ vector particles (vp) of the vector per kilogram (vp) of total body mass of the subject (vp/kg). In some embodiments, the vector is administered at a dose of between about 1×10¹³ and 5×10¹⁴ vp/kg. In some embodiments, the vector is administered at a dose of between about 5×10¹³ and 3×10¹⁴ vp/kg. In some embodiments, the vector is administered at a dose of between about 5×10¹³ and 1×10¹⁴ vp/kg. In some embodiments, the vector is administered at a dose of less than about 1×10¹² vp/kg, less than about 3×10¹² vp/kg, less than about 5×10¹² vp/kg, less than about 7×10¹² vp/kg, less than about 1×10¹³ vp/kg, less than about 3×10¹³ vp/kg, less than about 5×10¹³ vp/kg, less than about 7×10¹³ vp/kg, less than about 1×10¹⁴ vp/kg, less than about 3×10¹⁴ vp/kg, less than about 5×10¹⁴ vp/kg, less than about 7×10¹⁴ vp/kg, less than about 1×10¹⁵ vp/kg, less than about 3×10¹⁵ vp/kg, less than about 5×10¹⁵ vp/kg, or less than about 7×10¹⁵ vp/kg.

Antibody Dosage and Timing

The dose of the multispecific antibody (e.g., bispecific antibody) given to a subject having a disease or disorder (e.g., cancer, such as hematological malignancy) is sufficient to improve vector transduction efficiency as described herein (“an effective amount”). While a dose of multispecific antibody sufficient to induce a cytotoxic or other secondary therapeutic effect on cells could be used, more preferably a reduced dose is used, particularly where the non-target cell (e.g. B cell for a CD3×CD19 bispecific) is a non-malignant cell or a cell desirable for the therapeutic effect of the target immune cell (e.g. T cell) to be generated after transduction. In some embodiments, the dose selected is 10×, 100×, 1000×, 5000×, or 10000× lower than the dose of the multispecific antibody used in monotherapy. In some embodiments, the dose includes from about 5 μg to about 10 mg/kg, e.g. about 0.005 mg to about 3 mg/kg or about 0.5 mg to about 2.5 mg/kg, or about 0.4 mg/kg, about 0.8 mg/kg, about 1.6 mg/kg, or about 2.4 mg/kg of the antibody. Suitable doses include, for example, about 0.01, 0.02, 0.05, 0.07, 0.1, 0.2, 0.3, 0.4, 0.5, 0.6, 0.7, 0.8, 0.9, 1.0, 1.5, 1.6, 1.7, 1.8, 1.9, 2.0, 2.1, 2.2, 2.3, 2.4, 2.5, 3.0, 4.0, 5.0, 6.0, 7.0, 8.0, 9.0, or 10.0 mg/kg. In some embodiments, the dose includes from about 0.05 μg to about 1 mg/kg, e.g. about 0.5 μg to about 0.30 mg/kg or about 0.005 mg to about 0.25 mg/kg, or about 0.04 mg/kg, about 0.08 mg/kg, about 0.16 mg/kg, or about 0.24 mg/kg of the antibody. Suitable doses include, for example, about 0.001, 0.002, 0.005, 0.007, 0.01, 0.02, 0.03, 0.04, 0.05, 0.06, 0.07, 0.08, 0.09, 0.01, 0.015, 0.016, 0.017, 0.018, 0.019, 0.02, 0.021, 0.022, 0.023, 0.024, 0.025, 0.03, 0.04, 0.05, 0.06, 0.07, 0.08, 0.09, or 0.1 mg/kg.

A fixed unit dose of the multispecific antibody may also be given, for example, about 1, 2, 5, 10, 20, 50, 100, 200, 500, or 1000 mg, or the dose may be based on the patient's surface area, e.g., about 500, 400, 300, 250, 200, 100, 50, 20, 10, 5, 2, or 1 mg/m². In some embodiments, the fixed unit dose of the multispecific antibody is, for example, about 0.1, 0.2, 0.5, 0.1, 0.2, 0.5, 0.1, 0.2, 0.5, or 1 mg, or the dose may be based on the patient's surface area, e.g., about 50, 40, 30, 25, 20, 10, 5, 2, 1, 0.5, 0.2, or 0.1 mg/m². In some embodiments, the fixed unit dose of the multispecific antibody is, for example, about 0.01, 0.02, 0.05, 0.01, 0.02, 0.05, 0.01, 0.02, 0.05, or 0.1 mg, or the dose may be based on the patient's surface area, e.g., about 5, 4, 3, 2.5, 2, 1, 0.5, 0.2, 0.1, 0.05, 0.02, or 0.01 mg/m².

The multispecific antibody may be administered before, during, or after administration of the vector (e.g., viral vector). In some embodiments, the multispecific antibody is administered one week, 7 days, 6 days, 5 days, 4 days, 3 days, 2 days, or 1 day before the vector. In some embodiments, the multispecific antibody is administered 1-4 hours or 4-8 hours, or about 1 hour, about 2 hours, about 3 hours, or about 4 hours prior to administration of the vector. In some embodiments, the multispecific antibody is administered concurrent to administration of the vector. In some embodiments, the multispecific antibody is administered after the vector (e.g., 1-4 hours, 1-8 hours, or 1 day after the vector).

While multispecific antibodies in current therapeutic used are generally administered repeatedly (i.e. on a weekly, bi-weekly, or monthly schedule). In the methods of the present disclosure, the multispecific antibody may be administered as few as one time, two times, or three times. In particular embodiments, the administration of the multispecific antibody is performed exactly once. Similarly stated, a single injection of the multispecific antibody is administered prior to or concurrent to administration of the vector. Where repeated administration of the vector is desired, one may choose to repeat the administration protocol for the multispecific antibody each time treatment with the vector is performed.

Administration Route

In some embodiments, the vector is administered via a route selected from the group consisting of parenteral, intravenous, intramuscular, subcutaneous, intratumoral and intralymphatic. In some embodiments, the vector is administered multiple times. In some embodiments, the vector is administered by intralymphatic injection of the vector. In some embodiments, the vector is administered by injection of the vector into tumor sites (i.e. intratumoral). In some embodiments, the vector is administered subcutaneously. In some embodiments, the vector is administered systemically. In some embodiments, the vector is administered intravenously. In some embodiments, the vector is administered intra-arterially. In some embodiments, the vector is a lentiviral vector.

In some embodiments, the multispecific antibody is administered via a route selected from the group consisting of parenteral, intravenous, intramuscular, subcutaneous, intratumoral and intralymphatic. In some embodiments, the antibody is administered multiple times. In some embodiments, the antibody is administered by intralymphatic injection of the antibody. In some embodiments, the antibody is administered by injection of the antibody into tumor sites (i.e. intratumoral). In some embodiments, the antibody is administered subcutaneously. In some embodiments, the antibody is administered systemically. In some embodiments, the antibody is administered intravenously. In some embodiments, the antibody is administered intra-arterially. In some embodiments, the antibody is a bispecific antibody.

The multispecific antibody and the need not share the same mode of administration, e.g., a first agent (e.g., antibody) may be administrated systematically whereas a second agent (e.g., vector) may be administered intralymphatic.

Transduction Efficiency

In some embodiments, the compositions and methods of the present disclosure of using a multispecific antibody can increase the transduction efficiency of viral vectors by at least about 10%, at least about 20%, at least about 30%, at least about 40%, at least about 50%, at least about 60%, at least about 70%, at least about 80%, at least about 90%, at least about 100%, or more, as compared to the transduction efficiency of viral vectors without the use of such multispecific antibody.

In some embodiments, the compositions and methods of the present disclosure of using a multispecific antibody can increase the transduction efficiency of viral vectors by at least about 1-fold, at least about 2-fold, at least about 3-fold, at least about 5-fold, at least about 7-fold, at least about 10-fold, at least about 20-fold, at least about 30-fold, at least about 50-fold, at least about 70-fold, at least about 100-fold, at least about 200-fold, at least about 300-fold, at least about 500-fold, at least about 700-fold, at least about 1000-fold, or more, as compared to the transduction efficiency of viral vectors without the use of such multispecific antibody.

Combinatorial Therapy

The present disclosure further contemplates that one or more additional agents that improve the transduction efficiency of vector may be used in combination with the multispecific antibody and the vector described herein.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, dexamethasone, vincristine, cyclophosphamide, hydroxy daunorubicin, prednisone, rituximab, imatinib, dasatinib, nilotinib, bosutinib, ponatinib, bafetinib, saracatinib, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

### Vector

The vector can be a viral or non-viral vector. Illustrative non-viral vectors include, e.g., naked DNA, cationic liposome complexes, cationic polymer complexes, cationic liposome-polymer complexes, and exosomes. Examples of viral vectors include, but are not limited to, adenoviral, retroviral, lentiviral, herpesvirus and adeno-associated virus (AAV) vectors.

In some embodiments, the vector comprises a polynucleotide. In some embodiments, the polynucleotide encodes at least one therapeutic polypeptide. The term “therapeutic polypeptide” refers to a polypeptide which is being developed for therapeutic use, or which has been developed for therapeutic use. In some embodiments, the therapeutic polypeptide is expressed in target cells (e.g., host T cells) for therapeutic use. In some embodiments, the therapeutic polypeptide comprises a T cell receptor, a chimeric antigen receptor, or a cytokine receptor.

In some embodiments, the vector as described herein is a retroviral vector. In some embodiments, the vector is a lentiviral vector. In some embodiments, the vector is an adeno-associated virus vector.

The term viral vector may refer either to a vector or viral particle capable of transferring a nucleic acid into a cell or to the transferred nucleic acid itself. Viral vectors contain structural and/or functional genetic elements that are primarily derived from a virus. The term “retroviral vector” refers to a viral vector containing structural and functional genetic elements, or portions thereof, that are primarily derived from a retrovirus. The term “lentiviral vector” refers to a viral vector containing structural and functional genetic elements, or portions thereof, including LTRs that are primarily derived from a lentivirus. The term “hybrid” refers to a vector, LTR or other nucleic acid containing both retroviral, e.g., lentiviral, sequences and non-lentiviral viral sequences. In some embodiments, a hybrid vector refers to a vector or transfer plasmid comprising retroviral, e.g., lentiviral, sequences for reverse transcription, replication, integration and/or packaging.

Vector Type

Retroviral Vector

Retroviruses include lentiviruses, gamma-retrovirues, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Illustrative lentiviruses include, but are not limited to: HIV (human immunodeficiency virus; including HIV type 1, and HIV type 2; visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, the backbones are HIV-based vector backbones (i.e., HIV cis-acting sequence elements). Retroviral vectors have been generated by multiply attenuating the HIV virulence genes, for example, the genes env, vif, vpr, vpu and nef are deleted, making the vector biologically safe.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” refers to a viral particle that includes a polynucleotide encoding a heterologous protein (e.g. a chimeric antigen receptor), one or more capsid proteins, and other proteins necessary for transduction of the polynucleotide into a target cell. Retroviral particles and lentiviral particles generally include an RNA genome (derived from the transfer plasmid), a lipid-bilayer envelope in which the Env protein is embedded, and other accessory proteins including integrase, protease, and matrix protein.

The ex vivo efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of Third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to a hapten. In some embodiments, a sequence encoding a receptor that specifically binds to the hapten is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise a polynucleotide comprising a sequence encoding a T cell activator protein. In some embodiments, the retroviral particles comprise a polynucleotide comprising a sequence encoding a hapten-binding receptor. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to the hapten, and (iv) a 3′ LTR or UTR.

In some embodiments, the retroviral particles comprise a cell surface receptor that binds to a ligand on a target host cell, allowing host cell transduction. The viral vector may comprise a heterologous viral envelope glycoprotein giving a pseudotyped viral vector. For example, the viral envelope glycoprotein may be derived from RD 114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof. In some embodiments, the viral fusion glycoprotein comprises the amino acid sequence of SEQ ID NO: 33 (Cocal G protein). In some embodiments, the viral fusion glycoprotein comprises an amino acid sequence at least 95% identical to SEQ ID NO: 33 (Cocal G protein). In some embodiments, the viral fusion glycoprotein comprises an amino acid sequence at least 50%, at least 55%, at least 60%, at least 65%, at least 70%, at least 75%, at least 80%, at least 85%, at least 90%, at least 95%, at least 96%, at least 97%, at least 98%, or at least 99% identical to SEQ ID NO: 33 (Cocal G protein), as below:

(SEQ ID NO: 33)
NFLLLTFIVLPLCSHAKFSIVFPQSQKGNWKNVPSSYHYCPSSSDQNWHND
LLGITMKVKMPKTHKAIQADGWMCHAAKWITTCDFRWYGPKYITHSIHSIQ
PTSEQCKESIKQTKQGTWMSPGFPPQNCGYATVTDSVAVVVQATPHHVLVD
EYTGEWIDSQFPNGKCETEECETVHNSTVWYSDYKVTGLCDATLVDTEITF
FSEDGKKESIGKPNTGYRSNYFAYEKGDKVCKMNYCKHAGVRLPSGVWFEF
VDQDVYAAAKLPECPVGATISAPTQTSVDVSLILDVERILDYSLCQETWSK
IRSKQPVSPVDLSYLAPKNPGTGPAFTIINGTLKYFETRYIRIDIDNPIIS
KMVGKISGSQTERELWTEWFPYEGVEIGPNGILKTPTGYKFPLFMIGHGML
DSDLHKTSQAEVFEHPHLAEAPKQLPEEETLFFGDTGISKNPVELIEGWFS
SWKSTVVTFFFAIGVFILLYVVARIVIAVRYRYQGSNNKRIYNDIEMSRFR
K

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD 114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the vector is a Nipah virus (NiV) envelope pseudotyped lentivirus particle (“Nipah envelope pseudotyped vector”). In some embodiments, the Nipah envelope pseudotyped vector is pseudotyped using Nipah virus envelope glycoproteins NiV-F and NiV-G. In some embodiments, the NiV-F and/or NiV-G glycoproteins on such Nipah envelope pseudotyped vector are modified variants. In some embodiments, the NiV-F and/or NiV-G glycoproteins on such Nipah envelope pseudotyped vector are modified to include an antigen binding domain. In some embodiments, the antigen is EpCAM, CD4, or CD8. In some embodiments, the Nipah envelope pseudotyped vector can efficiently transduce cells expressing EpCAM, CD4, or CD8. See U.S. Pat. No. 9,486,539 and Bender et al. PLoS Pathog. 2016 June; 12(6): e1005641.

In some embodiments, the retroviral vector is surface-engineered. Illustrative methods of surface-engineering a retroviral vector are provided in, e.g., WO 2019/200056, PCT/US2019/062675, and U.S. 62/916,110, each of which is incorporated herein by reference in its entirety.

Various non-viral proteins capable of viral surface display are provided by the present disclosure. In some embodiments the non-viral proteins are co-stimulatory molecules. Conventionally, lentiviral transduction in vitro requires additional of an exogenous activating agent, such as a “stimbead,” for example Dynabeads™ Human T-Activator CD3/CD28. In some embodiments, the retroviral (e.g. lentiviral) vectors of the present disclosure incorporate one or more copies of non-viral proteins such as T-cell activation or co-stimulation molecule(s). The incorporation of T-cell activation or co-stimulation molecule(s) in the vector may render the vector capable of activating and efficiently transducing T cells in the absence of, or in the presence of lower amounts of, an exogenous activating agent, i.e. without a stimbead or equivalent agent. This permits the vectors further enhances in vivo transduction of T cells using a multispecific antibody according the method disclosed herein.

In some embodiments, the T-cell activation or co-stimulation molecule may be selected from the group consisting of an anti-CD3 antibody, CD28 ligand (CD28L), and 41bb ligand (41BBL or CD137L). Various T-cell activation or co-stimulation molecules are known in the art and include, without limitation, agents that specifically bind any of the T-cell expressed proteins CD3, CD28, CD134 also known as OX40, or 41bb also known as 4-1BB or CD137 or TNFRSF9. For example, an agent that specifically binds CD3 may be an anti-CD3 antibody (e.g., OKT3, CRIS-7 or I2C) or an antigen-binding fragment of an anti-CD3 antibody.

In some embodiments, an agent that specifically binds CD3 is a single chain Fv fragment (scFv) of an anti-CD3 antibody. In some embodiments, the T-cell activation or co-stimulation molecule is selected from the group consisting of an anti-CD3 antibody, a ligand for CD28 (e.g., CD28L), and 41bb ligand (41BBL or CD137L). CD86, also known as B7-2, is a ligand for both CD28 and CTLA-4. In some embodiments, the ligand for CD28 is CD86. CD80 is an additional ligand for CD28. In some embodiments, the ligand for CD28 is CD80. In some embodiments, the ligand for CD28 is an anti-CD28 antibody or an anti-CD28 scFv coupled to a transmembrane domain for display on the surface of the vector. Vectors comprising one or more a T-cell activation or co-stimulation molecule(s) may be made by engineering the packaging cell line by methods provided by WO 2016/139463; or by expression of the T-cell activation or co-stimulation molecule(s) from a polycistronic helper vector as described in PCT/US2019/062675.

In some embodiments, the vector comprises a ligand for CD19, or a functional fragment thereof, coupled to its native transmembrane domain or a heterologous transmembrane domain. In some embodiments, CD19 acts as a ligand for blinatumomab, thus providing an adapter for coupling the particle to T-cells via the anti-CD3 moiety of blinatumomab. In some embodiments, another type of particle surface ligand can serve to couple an appropriately surface engineered lentiviral particle to a T-cell using a multispecific antibody comprising a binding moiety for the particle surface ligand. In some embodiments, the multispecific antibody is a bispecific antibody, for example, a Bispecific T-cell engager (BiTE).

The non-viral protein may be a cytokine. In some embodiments, the cytokine may be selected from the group consisting of IL-15, IL-7, and IL-2. Where the non-viral protein used is a soluble protein (such as an scFv or a cytokine) it may be tethered to the surface of the lentiviral particle by fusion to a transmembrane domain, such as the transmembrane domain of CD8. Alternatively, it may be indirectly tethered to the lentiviral particle by use of a transmembrane protein engineered to bind the soluble protein. Further inclusion of one or more cytoplasmic residues may increase the stability of the fusion protein.

In some embodiments, the surface-engineered vector comprises a transmembrane protein comprising a mitogenic domain and/or cytokine-based domain. In particular embodiments, the mitogenic domain binds a T cell surface antigen, such as CD3, CD28, CD134 and CD137. In some embodiments, the mitogenic domain binds to a CD3E chain.

CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular).

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels.

CD137, also known as 4-1BB, is a member of the tumor necrosis factor (TNF) receptor family. CD137 can be expressed by activated T cells, but to a larger extent on CD8 than on CD4 T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity.

The mitogenic domain may comprise all or part of an antibody or other molecule which specifically binds a T-cell surface antigen. The antibody may activate the TCR or CD28. The antibody may bind the TCR, CD3 or CD28. Examples of such antibodies include: OKT3, 15E8 and TGN1412. Other suitable antibodies include:

Anti-CD28: CD28.2, 10F3

Anti-CD3/TCR: UCHT1, YTH12.5, TR66

The mitogenic domain may comprise the binding domain from OKT3, 15E8, TGN1412, CD28.2, 10F3, UCHT1, YTH12.5 or TR66.

The mitogenic domain may comprise all or part of a co-stimulatory molecule such as OX40L and 41 BBL. For example, the mitogenic domain may comprise the binding domain from OX40L or 41 BBL.

In some embodiments, the vector comprises an anti-CD3E antibody, or antigen-binding fragment thereof, coupled to a transmembrane domain. An illustrative anti-CD3E antibody is OKT3. OKT3, also known as Muromonab-CD3, is a monoclonal antibody targeted at the CD3E chain. It is clinically used to reduce acute rejection in patients with organ transplants. It was the first monoclonal antibody to be approved for clinical use in humans. The CDRs of OKT3 are as follows:

CDRH1:
(SEQ ID NO. 1)
GYTFTRY
CDRH2:
(SEQ ID NO. 2)
NPSRGY
CDRH3:
(SEQ ID NO. 3)
YYDDHYCLDY
CDRL1:
(SEQ ID NO. 4)
SASSSVSYMN
CDRL2:
(SEQ ID NO. 5)
DTSKLAS
CDRL3:
(SEQ ID NO. 6)
QQWSSNPFT

15E8 is a mouse monoclonal antibody to human CD28. Its CDRs are as follows:

CDRH1:
(SEQ ID NO. 7)
GFSLTSY
CDRH2:
(SEQ ID NO. 8)
WAGGS
CDRH3:
(SEQ ID NO. 9)
DKRAPGKLYYGYPDY
CDRL1:
(SEQ ID NO. 10)
RASESVEYYVTSLMQ
CDRL2:
(SEQ ID NO. 11)
AASNVES
CDRL3:
(SEQ ID NO. 12)
QQTRKVPST

In some embodiments, the vector comprises an anti-CD28 antibody, or antigen-binding fragment thereof, coupled to a transmembrane domain. TGN1412 (also known as CD28-SuperMAB) is a humanised monoclonal antibody that not only binds to, but is a strong agonist for, the CD28 receptor. Its CDRs are as follows.

CDRH1:
(SEQ ID NO. 13)
GYTFSY
CDRH2:
(SEQ ID NO. 14)
YPGNVN
CDRH3:
(SEQ ID NO. 15)
SHYGLDWNFDV
CDRL1:
(SEQ ID NO. 16)
HASQNIYVLN
CDRL2:
(SEQ ID NO. 17)
KASNLHT
CDRL3:
(SEQ ID NO: 18)
QQGQTYPYT

In some embodiments, the vector comprises a ligand for CD134, or functional fragment thereof, coupled to a transmembrane domain. OX40L is the native ligand for CD134 and is expressed on such cells as DC2s (a subtype of dendritic cells) enabling amplification of Th2 cell differentiation. OX40L has also been designated CD252 (cluster of differentiation 252).

The sequence of OX40L is:

(SEQ ID NO: 19)
MERVQPLEENVGNAARPRFERNKLLLVASVIQGLGLLLCFTYICLHFSALQ
VSHRYPRIQSIKVQFTEYKKEKGFILTSQKEDEIMKVQNYLISLKGYFSQE
VNISLHYQKDEEPLFQLKKVRSVNSLMVASLTYKDKVYLNVTTDNTSLDDF
HVNGGELILIHQNPGEFCVL

In some embodiments, the vector comprises a ligand for 4-1BB, or functional fragment thereof, coupled to its native transmembrane domain or a heterologous transmembrane domain. 4-1BBL is a cytokine that belongs to the tumor necrosis factor (TNF) ligand family. This transmembrane cytokine is a bidirectional signal transducer that acts as a ligand for 4-1BB, which is a costimulatory receptor molecule in T lymphocytes. 4-1BBL has been shown to reactivate anergic T lymphocytes in addition to promoting T lymphocyte proliferation.

The sequence of 41BBL is

(SEQ ID NO: 20)
MEYASDASLDPEAPWPPAPRARACRVLPWALVAGLLLLLLLAAACAVFLAC
PWAVSGARASPGSAASPRLREGPELSPDDPAGLLDLRQGMFAQLVAQNVLL
IDGPLSWYSDPGLAGVSLTGGLSYKEDTKELVVAKAGVYYVFFQLELRRVV
AGEGSGSVSLALHLQPLRSAAGAAALALTVDLPPASSEARNSAFGFQGRLL
HLSAGQRLGVHLHTEARARHAWQLTQGATVLGLFRVTPEIPAGLPSPRSE

Transduction Enhancer Spacer Domains

The mitogenic transduction enhancer and/or cytokine-based transduction enhancer may comprise a “spacer sequence” to connect the antigen-binding domain with the transmembrane domain. A flexible spacer allows the antigen-binding domain to orient in different directions to facilitate binding. As used herein, the term “coupled to” refers to a chemical linkage, a direct C-terminal to N-terminal fusion of two protein; chemical linkage to a non-peptide space; chemical linkage to a polypeptide space; and C-terminal to N-terminal fusion of two protein via peptide bonds to a polypeptide spacer, e.g. a spacer sequence.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs. In some embodiments, the spacer sequence may be derived from a human protein.

Examples of amino acid sequences for these spacers are given below.

hinge-CH2CH3 of human lgG1:
(SEQ ID NO: 21)
AEPKSPDKTHTCPPCPAPPVAGPSVFLFPPKPKDTLMIARTPEVTCWVDVS
HEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQG
NVFSCSVMHEALHNHYTQKSLSLSPGKKD
human CD8 stalk:
(SEQ ID NO: 22)
TTTPAPRPPTPAPTIASQPLSLRPEACRPAAGGAVHTRGLDFACDI
human 1gG1 hinge:
(SEQ ID NO: 23)
AEPKSPDKTHTCPPCPKDPK
CD2 ectodomain:
(SEQ ID NO: 24)
KEITNALETWGALGQDINLDIPSFQMSDDIDDIKWEKTSDKKKIAQFRKEK
ETFKEKDTYKLFKNGTLKIKHLKTDDQDIYKVSIYDTKGKNVLEKIFDLKI
QERVSKPKISWTCINTTLTCEVMNGTDPELNLYQDGKHLKLSQRVITHKWT
TSLSAKFKCTAGNKVSKESSVEPVSCPEKGLD
CD34 ectodomain:
(SEQ ID NO: 25)
SLDNNGTATPELPTQGTFSNVSTNVSYQETTTPSTLGSTSLHPVSQHGNEA
TTNITETTVKFTSTSVITSVYGNTNSSVQSQTSVISTVFTTPANVSTPETT
LKPSLSPGNVSDLSTTSTSLATSPTKPYTSSSPILSDIKAEIKCSGIREVK
LTQGICLEQNKTSSCAEFKKDRGEGLARVLCGEEQADADAGAQVCSLLLAQ
SEVRPQCLLLVLANRTEISSKLQLMKKHQSDLKKLGILDFTEQDVASHQSY
SQKT

The transmembrane domain is the sequence of the mitogenic transduction enhancer and/or cytokine-based transduction enhancer that spans the membrane. The transmembrane domain may comprise a hydrophobic alpha helix. The transmembrane domain may be derived from CD28. In some embodiments, the transmembrane domain is derived from a human protein.

An alternative option to a transmembrane domain is a membrane-targeting domain such as a GPI anchor. GPI anchoring is a post-translational modification which occurs in the endoplasmic reticulum. Preassembled GPI anchor precursors are transferred to proteins bearing a C-terminal GPI signal sequence. During processing, the GPI anchor replaces the GPI signal sequence and is linked to the target protein via an amide bond. The GPI anchor targets the mature protein to the membrane. In some embodiments, the present tagging protein comprises a GPI signal sequence.

The viral vector of the present disclosure may comprise a cytokine-based transduction enhancer in the viral envelope. In some embodiments, the cytokine-based transduction enhancer is derived from the host cell during viral vector production. In some embodiments, the cytokine-based transduction enhancer is made by the host cell and expressed at the cell surface. When the nascent viral vector buds from the host cell membrane, the cytokine-based transduction enhancer may be incorporated in the viral envelope as part of the packaging cell-derived lipid bilayer.

The cytokine-based transduction enhancer may comprise a cytokine domain and a transmembrane domain. It may have the structure C-S-TM, where C is the cytokine domain, S is an optional spacer domain (e.g. a spacer sequence) and TM is the transmembrane domain. The spacer domain and transmembrane domains are as defined above.

The cytokine domain may comprise a T-cell activating cytokine, such as from IL2, IL7 and IL15, or a functional fragment thereof. As used herein, a “functional fragment” of a cytokine is a fragment of a polypeptide that retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NIR suggests that the structure of IL2 comprises a bundle of 4 helices (termed A-D), flanked by 2 shorter helices and several poorly defined loops. Residues in helix A, and in the loop region between helices A and B, are important for receptor binding. The sequence of IL2 is:

(SEQ ID NO: 26)
MYRMQLLSCIALSLALVTNSAPTSSSTKKTQLQLEHLLLDLQMILNGINNY
KNPKLTRMLTFKFYMPKKATELKHLQCLEEELKPLEEVLNLAQSKNFHLRP
RDLISNINVIVLELKGSETTFMCEYADETATIVEFLNRWITFCQSIISTLT

IL7 is a cytokine that serves as a growth factor for early lymphoid cells of both B- and T-cell lineages. The sequence of IL7 is:

(SEQ ID NO: 27)
MFHVSFRYIFGLPPLILVLLPVASSDCDIEGKDGKQYESVLMVSIDQLLDS
MKEIGSNCLNNEFNFFKRHICDANKEGMFLFRAARKLRQFLKMNSTGDFDL
HLLKVSEGTTILLNCTGQVKGRKPAALGEAQPTKSLEENKSLKEQKKLNDL
CFLKRLLQEIKTCWNKILMGTKEH

IL15 is a cytokine with structural similarity to IL-2. Like IL-2, IL-15 binds to and signals through a complex composed of IL-2/IL-15 receptor beta chain and the common gamma chain. IL-15 is secreted by mononuclear phagocytes, and some other cells, following infection by virus(es). This cytokine induces cell proliferation of natural killer cells; cells of the innate immune system whose principal role is to kill virally infected cells. The sequence of IL-15 is:

(SEQ ID NO: 28)
MRISKPHLRSISIQCYLCLLLNSHFLTEAGIHVFILGCFSAGLPKTEANWV
NVISDLKKIEDLIQSMHIDATLYTESDVHPSCKVTAMKCFLLELQVISLES
GDASIHDTVENLIILANNSLSSNGNVTESGCKECEELEEKNIKEFLQSFVH
IVQMFINTS

The cytokine-based transduction enhancer may comprise one of the following sequences, or a functional fragment or variant thereof:

membrane-IL7:
(SEQ ID NO: 29)
MAHVSFRYIFGLPPLILVLLPVASSDCDIEGKDGKQYESVLMVSIDQLLDS
MKEIGSNCLNNEFNFFKRHICDANKEGMFLFRAARKLRQFLKMNSTGDFDL
HLLKVSEGTTILLNCTGQVKGRKPAALGEAQPTKSLEENKSLKEQKKLNDL
CFLKRLLQEIKTCWNKILMGTKEHSGGGSPAKPTTTPAPRPPTPAPTIASQ
PLSLRPEACRPAAGGAVHTRGLDFACDIYIWAPLAGTCGVLLLSLVITLYC
NHRNRRRVCKCPRPVV
membrane-IL15:
(SEQ ID NO: 30)
MGLVRRGARAGPRMPRGWTALCLLSLLPSGFMAGIHVFILGCFSAGLPKTE
ANWVNVISDLKKIEDLIQSMHIDATLYTESDVHPSCKVTAMKCFLLELQVI
SLESGDASIHDTVENLIILANNSLSSNGNVTESGCKECEELEEKNIKEFLQ
SFVHIVQMFINTSSPAKPTTTPAPRPPTPAPTIASQPLSLRPEACRPAAGG
AVHTRGLDFACDIYIWAPLAGTCGVLLLSLVITLYCNHRNRRRVCKCPRPV
V

The cytokine-based transduction enhancer may comprise a variant of the sequence shown as SEQ ID NO: 29 or 30 having at least 80, 85, 90, 95, 98 or 99% sequence identity, provided that the variant sequence is a cytokine-based transduction enhancer having the required properties, i.e. the capacity to activate a T cell when present in the envelope protein of a retroviral or lentiviral vector.

The disclosure further provides various retroviral vectors, including but not limited to gamma-retroviral vectors, alpha-retroviral vectors, and lentiviral vectors.

AAV

In some embodiments, the viral vector is an adeno-associated virus (AAV) vector. AAV is a 4.7 kb, single stranded DNA virus. Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues. By an “AAV vector” is meant a vector derived from an adeno-associated virus serotype, including without limitation, AAV1, AAV2, AAV3, AAV4, AAV5, AAV6, AAV7, AAV8, AAV9, AAV10, AAVrh.10, AAVrh.74, etc. AAV vectors can have one or more of the AAV wild-type genes deleted in whole or part, e.g., the rep and/or cap genes, but retain functional flanking inverted terminal repeat (ITR) sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g. by the insertion, deletion or substitution of nucleotides, as long as the sequences provide for functional rescue, replication and packaging. AAV vectors may comprise other modifications, including but not limited to one or more modified capsid protein (e.g., VP1, VP2 and/or VP3). For example, a capsid protein may be modified to alter tropism and/or reduce immunogenicity.

Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues. Various serotypes of AAV are known, including, AAV1, AAV2, AAV3, AAV4, AAV5, AAV6, AAV7, AAV8, AAV9, AAV10, AAVrh.10, AAVrh.74, etc. AAV vectors can have one or more of the AAV wild-type genes deleted in whole or part, e.g., the rep and/or cap genes, but retain functional flanking inverted terminal repeat (ITR) sequences. The serotype of a recombinant AAV vector is determined by its capsid. International Patent Publication No. WO2003042397A2 discloses various capsid sequences including those of AAV1, AAV2, AAV3, AAV8, AAV9, and rh10. International Patent Publication No. WO2013078316A1 discloses the polypeptide sequence of the VP1 from AAVrh74. Numerous diverse naturally occurring or genetically modified AAV capsid sequences are known in the art.

AAV vectors useful in the practice of the present disclosure can be packaged into AAV virions (viral particles) using various systems including adenovirus-based and helper-free systems. Standard methods in AAV biology include those described in Kwon and Schaffer. Pharm Res. (2008) 25(3):489-99; Wu et al. Mol. Ther. (2006) 14(3):316-27. Burger et al. Mol. Ther. (2004) 10(2):302-17; Grimm et al. Curr Gene Ther. (2003) 3(4):281-304; Deyle D R, Russell D W. Curr OpinMol Ther. (2009) 11(4):442-447; McCarty et al. Gene Ther. (2001) 8(16):1248-54; and Duan et al. Mol Ther. (2001) 4(4):383-91. Helper-free systems included those described in U.S. Pat. Nos. 6,004,797; 7,588,772; and 7,094,604.

Gene delivery viral vectors useful in the practice of the present disclosure can be constructed utilizing methodologies known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

Illustrative examples of viral vectors usable in the compositions and methods of the present disclosure are disclosed in WO2016/139463; WO2017/165245; WO2018111834; each of which is incorporated herein in its entirety.

Non-Viral Vector

In some embodiments, the compositions and methods of the disclosure can be used with a non-viral vector. Illustrative non-viral vectors are provided in, e.g., Smith et al. Nat Nanotechnol. 12(8):813-820 (2017). In some embodiments, the non-viral vector is a type of nanoparticles. In some embodiments, the nanoparticle is polymer based. In some embodiments, the non-viral vector is liposome based. In some embodiments, the nanoparticle is equipped with immune-cell targeting molecules. In some embodiments, the nanoparticle is loaded with polynucleotide molecules encoding one or more expression cassette.

Chimeric Antigen Receptors

In some embodiments, the vector described herein are used to transduce a nucleic acid sequence (polynucleotide) encoding one or more chimeric antigen receptor (CARs) into a cell (e.g., a T lymphocyte). In some embodiments, the transduction of the vector results in expression of one or more CARs in the transduced cells.

CARs are artificial membrane-bound proteins that direct a T lymphocyte to an antigen, and stimulate the T lymphocyte to kill cells displaying the antigen. See, e.g., Eshhar, U.S. Pat. No. 7,741,465. Generally, CARs are genetically engineered receptors comprising an extracellular domain that binds to an antigen, e.g., an antigen on a cell, an optional linker, a transmembrane domain, and an intracellular (cytoplasmic) domain comprising a costimulatory domain and/or a signaling domain that transmits an activation signal to an immune cell. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell. All other conditions being satisfied, when a CAR is expressed on the surface of, e.g., a T lymphocyte, and the extracellular domain of the CAR binds to an antigen, the intracellular signaling domain transmits a signal to the T lymphocyte to activate and/or proliferate, and, if the antigen is present on a cell surface, to kill the cell expressing the antigen. Because T lymphocytes require two signals, a primary activation signal and a costimulatory signal, in order to maximally activate, CARs can comprise a stimulatory and a costimulatory domain such that binding of the antigen to the extracellular domain results in transmission of both a primary activation signal and a costimulatory signal.

CAR Intracellular Domain

In some embodiments, the intracellular domain of the CAR is or comprises an intracellular domain or motif of a protein that is expressed on the surface of T lymphocytes and triggers activation and/or proliferation of said T lymphocytes. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In some embodiments, the intracellular domain is a CD3ζ intracellular signaling domain. In some embodiments, the intracellular domain is from a lymphocyte receptor chain, a TCR/CD3 complex protein, an Fc receptor subunit or an IL-2 receptor subunit. In some embodiments, the intracellular signaling domain of CAR may be derived from the signaling domains of for example OO3ζ, CD3ε, CD22, CD79a, CD66d or CD39. “Intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

In some embodiments, the CAR additionally comprises one or more co-stimulatory domains or motifs, e.g., as part of the intracellular domain of the polypeptide. Co-stimulatory molecules are well-known cell surface molecules other than antigen receptors or Fc receptors that provide a second signal required for efficient activation and function of T lymphocytes upon binding to antigen. The one or more co-stimulatory domains or motifs can, for example, be, or comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof. In some embodiments, the one or more co-stimulatory domains are selected from the group consisting of intracellular domains of 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, and ZAP70.

In some embodiments, the intracellular domain can be further modified to encode a detectable, for example, a fluorescent, protein (e.g., green fluorescent protein) or any variants known thereof.

CAR Transmembrane Region

The transmembrane region can be any transmembrane region that can be incorporated into a functional CAR, e.g., a transmembrane region from a CD4 or a CD8 molecule.

In some embodiments, The transmembrane domain of CAR may be derived from the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), 4-1 BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

CAR Linker Region

The optional linker of CAR positioned between the extracellular domain and the transmembrane domain may be a polypeptide of about 2 to 100 amino acids in length. The linker can include or be composed of flexible residues such as glycine and serine so that the adjacent protein domains are free to move relative to one another. Longer linkers may be used, e.g., when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linker is derived from a hinge region or portion of the hinge region of any immunoglobulin.

CAR Extracellular Domain

In some embodiments, the nucleic acid transduced into cells using the methods described herein comprises a sequence that encodes a polypeptide, wherein the extracellular domain of the polypeptide binds to an antigen of interest. In some embodiments the extracellular domain comprises a receptor, or a portion of a receptor, that binds to said antigen. In some embodiments, the extracellular domain comprises, or is, an antibody or an antigen-binding portion thereof. In some embodiments, the extracellular domain comprises, or is, a single-chain Fv domain. The single-chain Fv domain can comprise, for example, a VL linked to VH by a flexible linker, wherein said VL and VH are from an antibody that binds said antigen.

In some embodiments, the extracellular domain of CAR may contain any polypeptide that binds the desired antigen (e.g. prostate neoantigen). The extracellular domain may comprise a scFv, a portion of an antibody or an alternative scaffold. CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In some embodiments, said lymphoma can be chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma. In some embodiments, in which the cancer is chronic lymphocytic leukemia (CLL), the B cells of the CLL have a normal karyotype. In some embodiments, in which the cancer is chronic lymphocytic leukemia (CLL), the B cells of the CLL carry a 17p deletion, an 11q deletion, a 12q trisomy, a 13q deletion or a p53 deletion.

In some embodiments, the antigen is a tumor-associated antigen (TAA) or a tumor-specific antigen (TSA). In some embodiments, without limitation, the tumor-associated antigen or tumor-specific antigen is B cell maturation antigen (BCMA), B cell Activating Factor (BAFF), Her2, prostate stem cell antigen (PSCA), prostate-specific membrane antigen (PSMA) alpha-fetoprotein (AFP), carcinoembryonic antigen (CEA), EGFRvIII, cancer antigen-125 (CA-125), CA19-9, calretinin, WUC-1, epithelial membrane protein (EMA), epithelial tumor antigen (ETA), tyrosinase, melanoma-associated antigen (MAGE), CD19, CD20, CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, vascular endothelial growth factor receptor (VEGFR), the dimeric form of the pyruvate kinase isoenzyme type M2 (tumor M2-PK), an abnormal ras protein, or an abnormal p53 protein.

In some embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In some embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In some embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAAO205, Mart2, Mum-1, 2, and 3, neo-PAP, myosin class I, OS-9, pml-RARu fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, pi5(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB70\K, NY-CO-1, RCAS1, SDCCAGi6, TA-90, TAAL6, TAG72, TLP, TPS, CD19, CD22, CD27, CD30, CD70, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In some embodiments, said tumor-associated antigen or tumor-specific antigen is integrin αvβ (CD61), galactin, K-Ras (V-Ki-ras2 Kirsten rat sarcoma viral oncogene), or Ral-B. Other tumor-associated and tumor-specific antigens are known to those in the art.

Antibodies, and scFvs, that bind to TSAs and TAAs include antibodies and scFVs that are known in the art, as are nucleotide sequences that encode them.

In some embodiments, the antigen is an antigen not considered to be a TSA or a TAA, but which is nevertheless associated with tumor cells, or damage caused by a tumor. In some embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in some embodiments, the antigen is a hypoxia-associated factor, e.g., HIF-1α, HIF-1β, HIF-2a, HIF-2β, HIF-3α, or HIF-3β. Tumors can also cause localized damage to normal tissue, causing the release of molecules known as damage associated molecular pattern molecules (DAMPs; also known as alarmins). In some embodiments, therefore, the antigen is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

In some embodiments of the polypeptides described herein, the extracellular domain is joined to said transmembrane domain directly or by a linker, spacer or hinge polypeptide sequence, e.g., a sequence from CD28 or a sequence from CTLA4.

In some embodiments, the extracellular domain that binds the desired antigen may be derived from antibodies or their antigen binding fragments generated using the technologies described herein.

Examples of CAR

Non-limiting examples of chimeric antigen receptor that can be used in conjunction with the compositions and methods of the present disclosure are disclosed in WO 2019/200056; PCT/US2019/062675; U.S. 62/916,110; WO2015/017214; WO/2018/148224; WO2019156795, each of which is incorporated herein in its entirety.

Gene Editing

Numerous gene-editing methods are known in the art and additional methods are continuously being created. The methods and compositions of the present disclosure are capable of delivering a variety of genetic payloads, including polynucleotides intended for insertion into the genome of the target cell and/or gene editing systems (CRISPR-Cas, meganucleases, homing endonucleases, zinc finger enzymes and the like). In embodiments, a polynucleotide (e.g. transgene), enzyme, and/or guide RNA are delivered in one, two, three or more vectors of the same type (e.g. lentivirus, AAV, etc.) or different types (including e.g. combinations of non-viral and virus vectors or different types of viral vectors). The methods and systems of the disclosure can be used for generating point mutation(s), insertions, deletions, etc. Random mutagenesis and multi-locus gene editing are also within the scope of the disclosure.

### Target Immune Cells

Non-limiting examples of cells that can be the target of vector described herein include T lymphocytes, dendritic cells (DC), Treg cells, B cells, Natural Killer cells, macrophages . . . .

T Cells

T cells (“T lymphocytes”) are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MIHC complex), the T lymphocyte is activated through a series of biochemical events mediated by associated enzymes, co-receptors, specialized adaptor molecules, and activated or released transcription factors.

In some embodiments, the cells used in the methods provided herein are primary T lymphocytes (e.g., primary human T lymphocytes). The primary T lymphocytes used in the methods provided herein may be naive T lymphocytes or MHC-restricted T lymphocytes. In some embodiments, the T lymphocytes are CD4⁺. In other embodiments, the T lymphocytes are CD8⁺. In some embodiments, the primary T lymphocytes are tumor infiltrating lymphocytes (TILs). In some embodiments, the primary T lymphocytes have been isolated from a tumor biopsy, or have been expanded from T lymphocytes isolated from a tumor biopsy. In some embodiments, the primary T lymphocytes have been isolated from, or are expanded from T lymphocytes isolated from, peripheral blood, cord blood, or lymph. In some embodiments, the T lymphocytes are allogeneic with respect to a particular individual, e.g., a recipient of said T lymphocytes. In certain other embodiments, the T lymphocytes are not allogeneic with respect to a certain individual, e.g., a recipient of said T lymphocytes. In some embodiments, the T lymphocytes are autologous with respect to a particular individual, e.g., a recipient of said T lymphocytes.

In some embodiments, primary T lymphocytes used in the methods described herein are isolated from a tumor, e.g., are tumor-infiltrating lymphocytes. In some embodiments, such T lymphocytes are specific for a tumor specific antigen (TSA) or tumor associated antigen (TAA). In some embodiments, primary T lymphocytes are obtained from an individual, optionally expanded, and then transduced, using the methods described herein, with a nucleic acid encoding one or more chimeric antigen receptors (CARs), and optionally then expanded. T lymphocytes can be expanded, for example, by contacting the T lymphocytes in culture with antibodies to CD3 and/or CD28, e.g., antibodies attached to beads, or to the surface of a cell culture plate; see, e.g., U.S. Pat. Nos. 5,948,893; 6,534,055; 6,352,694; 6,692,964; 6,887,466; and 6,905,681. In some embodiments, the antibodies are anti-CD3 and/or anti-CD28, and the antibodies are not bound to a solid surface (e.g., the antibodies contact the T lymphocytes in solution). In some embodiments, either of the anti-CD3 antibody or anti-CD28 antibody is bound to a solid surface (e.g. bead, tissue culture dish plastic), and the other antibody is not bound to a solid surface (e.g., is present in solution).

NK Cells

Natural killer (NK) cells are cytotoxic lymphocytes that constitute a major component of the innate immune system. NK cells typically comprise approximately 10 to 15% of the mononuclear cell fraction in normal peripheral blood. NK cells do not express T-cell antigen receptors (TCR), CD3 or surface immunoglobulins (Ig) B cell receptor, but usually express the surface markers CD16 (FcγRIII) and CD56 in humans. NK cells are cytotoxic; small granules in their cytoplasm contain special proteins such as perform and proteases known as granzymes. Upon release in close proximity to a cell slated for killing, perform forms pores in the cell membrane of the target cell through which the granzymes and associated molecules can enter, inducing apoptosis. One granzyme, granzyme B (also known as granzyme 2 and cytotoxic T-lymphocyte-associated serine esterase 1), is a serine protease crucial for rapid induction of target cell apoptosis in the cell-mediated immune response.

NK cells are activated in response to interferons or macrophage-derived cytokines Activated NK cells are referred to as lymphokine activated killer (LAK) cells. NK cells possess two types of surface receptors, labeled “activating receptors” and “inhibitory receptors,” that control the cells' cytotoxic activity.

Among other activities, NK cells play a role in the host rejection of tumors. Because many cancer cells have reduced or no class I MHC expression, they can become targets of NK cells. Natural killer cells can become activated by cells lacking, or displaying reduced levels of, major histocompatibility complex (MHC) proteins. In addition to being involved in direct cytotoxic killing, NK cells also serve a role in cytokine production, which can be important to control cancer and infection. Activated and expanded NK cells and LAK cells have been used in both ex vivo therapy and in vivo treatment of patients having advanced cancer, with some success against bone marrow related diseases, such as leukemia; breast cancer; and certain types of lymphoma.

### Pharmaceutical Compositions and Formulations

The formulations and compositions of the present disclosure may comprise a combination of any number of multispecific antibodies and/or vectors, and optionally one or more additional pharmaceutical agent (polypeptides, polynucleotides, compounds etc.) formulated in pharmaceutically acceptable or physiologically-acceptable compositions for administration to a cell, tissue, organ, or an animal, either alone, or in combination with one or more other modalities of therapy. In some embodiments, the one or more additional pharmaceutical agent further increases transduction efficiency of vectors.

In some embodiments, the present disclosure provides compositions comprising a therapeutically-effective amount of a multispecific antibody (e.g. a bispecific antibody), as described herein, formulated together with one or more pharmaceutically acceptable carriers (additives) and/or diluents. In some embodiments, the composition further comprises other agents, such as, e.g., cytokines, growth factors, hormones, small molecules or various pharmaceutically active agents.

In some embodiments, compositions and formulations of the antibodies used in accordance with the present disclosure may be prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed. In some embodiments, one or more pharmaceutically acceptable surface-active agents (surfactant), buffers, isotonicity agents, salts, amino acids, sugars, stabilizers and/or antioxidant are used in the formulation.

Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Suitable buffer comprise but are not limited to histidine-buffers, citrate-buffers, succinate-buffers, acetate-buffers and phosphate-buffers.

Isotonicity agents are used to provide an isotonic formulation. An isotonic formulation is liquid or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. In some embodiments, isotonicity agents are generally used in a total amount of about 5 mM to about 350 mM.

Non-limiting examples of salts include salts of any combinations of the cations sodium potassium, calcium or magnesium with anions chloride, phosphate, citrate, succinate, sulphate or mixtures thereof. Non-limiting examples of amino acids comprise arginine, glycine, ornithine, lysine, histidine, glutamic acid, asparagic acid, isoleucine, leucine, alanine, phenylalanine, tyrosine, tryptophane, methionine, serine, proline. Non-limiting examples of sugars according to the disclosure include trehalose, sucrose, mannitol, sorbitol, lactose, glucose, mannose, maltose, galactose, fructose, sorbose, raffinose, glucosamine, N-methylglucosamine (also referred to as “meglumine”), galactosamine and neuraminic acid and combinations thereof. Non-limiting examples of stabilizer includes amino acids and sugars as described above as well as commercially available cyclodextrins and dextrans of any kind and molecular weight as known in the art. Non-limiting examples of antioxidants include excipients such as methionine, benzylalcohol or any other excipient used to minimize oxidation.

In some embodiments, the present disclosure provides compositions comprising a therapeutically effective amount of a vector, as described herein, formulated together with one or more pharmaceutically acceptable carriers (additives) and/or diluents (e.g., pharmaceutically acceptable cell culture medium). In some embodiments, the composition further comprises other agents, such as, e.g., cytokines, growth factors, hormones, small molecules or various pharmaceutically active agents.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the present disclosure is contemplated.

The compositions may further comprise one or more polypeptides, polynucleotides, vectors comprising same, compounds that increase the transduction efficiency of vectors, formulated in pharmaceutically acceptable or physiologically-acceptable solutions for administration to a cell or an animal, either alone, or in combination with one or more other modalities of therapy. It will also be understood that, if desired, the compositions of the present disclosure may be administered in combination with other agents as well, such as, e.g., cytokines, growth factors, hormones, small molecules or various pharmaceutically active agents. There is virtually no limit to other components that may also be included in the compositions, provided that the additional agents do not adversely affect the ability of the composition to deliver the intended therapy.

The present disclosure also provides pharmaceutical compositions comprising an expression cassette or vector (e.g., therapeutic vector) disclosed herein and one or more pharmaceutically acceptable carriers, diluents or excipients. In some embodiments, the pharmaceutical composition comprises a lentiviral vector comprising an expression cassette disclosed herein, e.g., wherein the expression cassette comprises one or more polynucleotide sequences encoding one or more chimeric antigen receptor (CARs) and variants thereof.

The pharmaceutical compositions that contain the expression cassette or vector may be in any form that is suitable for the selected mode of administration, for example, for intraventricular, intramyocardial, intracoronary, intravenous, intra-arterial, intra-renal, intraurethral, epidural, intrathecal or intramuscular administration. The vector can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the vectors according to the present disclosure.

In some embodiments, the vector (e.g. lentiviral vector), or a pharmaceutical composition comprising that vector, is effective when administered systemically. For example, the viral vectors of the disclosure, in some cases, demonstrate efficacy when administered intravenously to subject (e.g., a primate, such as a non-human primate or a human). In some embodiments, the viral vectors of the disclosure are capable of inducing expression of CAR in various immune cells when administered systemically (e.g., in T-cells, dendritic cells, NK cells).

In various embodiments, the pharmaceutical compositions contain vehicles (e.g., carriers, diluents and excipients) that are pharmaceutically acceptable for a formulation capable of being injected. Exemplary excipients include a poloxamer. Formulation buffers for viral vectors general contains salts to prevent aggregation and other excipients (e.g. poloxamer) to reduce stickiness of the vector. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In some embodiments, the formulation is stable for storage and use when frozen (e.g. at less than 0° C., about −60° C., or about −72° C.).

The pharmaceutical compositions of the present disclosure, formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain circumstances it will be desirable to deliver the compositions disclosed herein parenterally, intravenously, intramuscularly, or intraperitoneally, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In some embodiments, isotonic agents, for example, sugars or sodium chloride, are added. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2005). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

In some embodiments, the present disclosure provides formulations or compositions suitable for the delivery of viral vector systems (i.e., viral-mediated transduction) including, but not limited to, retroviral (e.g., lentiviral) vectors.

### Diseases

The disclosure also provides a method of enhancing efficacy of an immunotherapy that can be used for treatment of diseases, disorders or conditions. In some embodiments, the disease or disorder is cancer. In some embodiments, the cancer is a hematological malignancy or a solid tumor. In some embodiments, the subject is relapsed or refractory to treatment with a prior anti-cancer therapeutic.

Hematological Malignancy

In some embodiments, the cancer is a hematological malignancy.

In some embodiments, the hematological malignancy is lymphoma, a B cell malignancy, Hodgkin's lymphoma, non-Hodgkin's lymphoma, a DLBLC, a FL, a MCL, a marginal zone B-cell lymphoma (MZL), a mucosa-associated lymphatic tissue lymphoma (MALT), a CLL, an ALL, an AML, Waldenstrom's Macroglobulinemia or a T-cell lymphoma.

In some embodiments, the solid tumor is a lung cancer, a liver cancer, a cervical cancer, a colon cancer, a breast cancer, an ovarian cancer, a pancreatic cancer, a melanoma, a glioblastoma, a prostate cancer, an esophageal cancer or a gastric cancer. WO2019057124A1 discloses cancers that are amenable to treatment with T cell redirecting therapeutics that bind CD19.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In some embodiments, the hematological malignancy is the multiple myeloma.

In some embodiments, the multiple myeloma is a newly diagnosed multiple myeloma.

In some embodiments, the multiple myeloma is a relapsed or a refractory multiple myeloma.

In some embodiments, the multiple myeloma is a high-risk multiple myeloma. Subjects with high-risk multiple myeloma are known to relapse early and have poor prognosis and outcome.

Subjects can be classified as having high-risk multiple myeloma is they have one or more of the following cytogenetic abnormalities: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p, or t(4;14)(p16;q32), t(14;16)(q32;q23) and del17p.

In some embodiments, the subject having the high-risk multiple myeloma has one or more chromosomal abnormalities comprising: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p; or t(4;14)(p16;q32), t(14;16)(q32;q23) and del17p, or any combination thereof.

Various qualitative and/or quantitative methods may be used to determine relapse or refractory nature of the disease. Symptoms that may be associated are for example a decline or plateau of the well-being of the patient or re-establishment or worsening of various symptoms associated with solid tumors, and/or the spread of cancerous cells in the body from one location to other organs, tissues or cells.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

In some embodiments, the multiple myeloma is relapsed or refractory to treatment with the anti-CD38 antibody, lenalinomide, bortezomib, pomalidomide, carfilzomib, elotozumab, ixazomib, melphalan or thalidomide, or any combination thereof.

In some embodiments, the hematological malignancy is the AML.

In some embodiments, the AML is AML with at least one genetic abnormality, AML with multilineage dysplasia, therapy-related AML, undifferentiated AML, AML with minimal maturation, AML with maturation, acute myelomonocytic leukemia, acute monocytic leukemia, acute erythroid leukemia, acute megakaryoblastic leukemia, acute basophilic leukemia, acute panmyelosis with fibrosis or myeloid sarcoma.

In some embodiments, the AML is AML with at least one genetic abnormality. In some embodiments, the AML is AML with multilineage dysplasia. In some embodiments, the AML is therapy-related AML. In some embodiments, the AML is undifferentiated AML. In some embodiments, the AML is AML with minimal maturation. In some embodiments, the AML is AML with maturation. In some embodiments, the AML is acute myelomonocytic leukemia. In some embodiments, the AML is acute monocytic leukemia. In some embodiments, the AML is acute erythroid leukemia. In some embodiments, the AML is acute megakaryoblastic leukemia. In some embodiments, the AML is acute basophilic leukemia. In some embodiments, the AML is acute panmyelosis with fibrosis. In some embodiments, the AML is myeloid sarcoma.

In some embodiments, the at least one genetic abnormality is a translocation between chromosomes 8 and 21, a translocation or an inversion in chromosome 16, a translocation between chromosomes 15 and 17, changes in chromosome 11, or mutation in fins-related tyrosine kinase 3 (FLT3), nucleophosmin (NPM1), isocitrate dehydrogenase 1 (IDH1), isocitrate dehydrogenase 2 (IDH2), DNA (cytosine-5)-methyltransferase 3 (DNMT3A), CCAAT/enhancer binding protein alpha (CEBPA), U2 small nuclear RNA auxiliary factor 1 (U2AF1), enhancer of zeste 2 polycomb repressive complex 2 subunit (EZH2), structural maintenance of chromosomes 1A (SMC1A) or structural maintenance of chromosomes 3 (SMC3).

In some embodiments, the at least one genetic abnormality is the translocation between chromosomes 8 and 21. In some embodiments, the at least one genetic abnormality is the translocation or an inversion in chromosome 16. In some embodiments, the at least one genetic abnormality is the translocation between chromosomes 15 and 17. In some embodiments, the at least one genetic abnormality is changes in chromosome 11. In some embodiments, the at least one genetic abnormality is the mutation in fins-related tyrosine kinase 3 (FLT3). In some embodiments, the at least one genetic abnormality is the mutation in nucleophosmin (NPM1). In some embodiments, the at least one genetic abnormality is the mutation in isocitrate dehydrogenase 1 (IDH1). In some embodiments, the at least one genetic abnormality is the mutation in isocitrate dehydrogenase 2 (IDH2). In some embodiments, the at least one genetic abnormality is the mutation in DNA (cytosine-5)-methyltransferase 3 (DNMT3A). In some embodiments, the at least one genetic abnormality is the mutation in CCAAT/enhancer binding protein alpha (CEBPA). In some embodiments, the at least one genetic abnormality is the mutation in U2 small nuclear RNA auxiliary factor 1 (U2AF1). In some embodiments, the at least one genetic abnormality is the mutation in enhancer of zeste 2 polycomb repressive complex 2 subunit (EZH2). In some embodiments, the at least one genetic abnormality is the mutation in structural maintenance of chromosomes 1A (SMC1A). In some embodiments, the at least one genetic abnormality is the mutation in structural maintenance of chromosomes 3 (SMC3).

In some embodiments, the at least one genetic abnormality is a translocation t(8; 21)(q22; q22), an inversion inv(16)(p13; q22), a translocation t(16; 16)(p13; q22), a translocation t(15; 17)(q22; q12), a mutation FLT3-ITD, mutations R132H or R100Q/R104V/F108L/R119Q/I130V in IDH1 or mutations R140Q or R172 in IDH2.

In some embodiments, the at least one genetic abnormality is the translocation t(8; 21)(q22; q22). In some embodiments, the at least one genetic abnormality is the inversion inv(16)(p13; q22). In some embodiments, the at least one genetic abnormality is the translocation t(16; 16)(p13; q22). In some embodiments, the at least one genetic abnormality is the translocation t(15; 17)(q22; q12). In some embodiments, the at least one genetic abnormality is the mutation FLT3-ITD. In some embodiments, the at least one genetic abnormality is the mutation R132H in IDH1. In some embodiments, the at least one genetic abnormality is the mutation R100Q/R104V/F108L/R119Q/I130V in IDH1. In some embodiments, the at least one genetic abnormality is the mutation R140Q in IDH2. In some embodiments, the at least one genetic abnormality is the mutation R172 in IDH2.

In some embodiments, the hematological malignancy is the ALL.

In some embodiments, the ALL is B-cell lineage ALL, T-cell lineage ALL, adult ALL or pediatric ALL.

In some embodiments, the ALL is B-cell lineage ALL. In some embodiments, the ALL is T-cell lineage ALL. In some embodiments, the ALL is adult ALL. In some embodiments, the ALL is pediatric ALL.

In some embodiments, the subject with ALL has a Philadelphia chromosome or is resistant or has acquired resistance to treatment with a BCR-ABL kinase inhibitor.

In some embodiments, the subject with ALL has the Philadelphia chromosome. In some embodiments, the subject with ALL is resistant or has acquired resistance to treatment with a BCR-ABL kinase inhibitor.

The Ph chromosome is present in about 20% of adults with ALL and a small percentage of children with ALL and is associated with poor prognosis. At a time of relapse, patients with Ph+ positive ALL may be on tyrosine kinase inhibitor (TKI) regimen and may have therefore become resistant to the TKI. The method as described herein may thus be administered to a subject who has become resistant to selective or partially selective BCR-ABL inhibitors. Exemplary BCR-ABL inhibitors are for example imatinib, dasatinib, nilotinib, bosutinib, ponatinib, bafetinib, saracatinib, tozasertib or danusertib.

Other chromosomal rearrangements identified in B-lineage ALL patients are t(v;11q23) (MLL rearranged), t(1;19)(q23;p13.3); TCF3-PBX1 (E2A-PBX1), t(12;21)(p13;q22); ETV6-RUNX1 (TEL-AML1) and t(5;14)(q31;q32); IL3-IGH.

In some embodiments, the subject has ALL with t(v;11q23) (MLL rearranged), t(1;19)(q23;p13.3); TCF3-PBX1 (E2A-PBX1), t(12;21)(p13;q22); ETV6-RUNX1 (TEL-AML1) or t(5;14)(q31;q32); IL3-IGH chromosomal rearrangement.

Chromosomal rearrangements can be identified using well known methods, for example fluorescent in situ hybridization, karyotyping, pulsed field gel electrophoresis, or sequencing.

In some embodiments, the hematological malignancy is the smoldering multiple myeloma, MGUS, ALL, DLBLC, BL, FL, MCL, Waldenstrom's macroglobulinema, plasma cell leukemia, AL, precursor B-cell lymphoblastic leukemia, precursor B-cell lymphoblastic leukemia, myelodysplastic syndrome (MDS), CLL, B cell malignancy, CML, HCL, blastic plasmacytoid dendritic cell neoplasm, Hodgkin's lymphoma, non-Hodgkin's lymphoma, MZL, MALT, plasma cell leukemia, ALCL, leukemia, or lymphoma.

Solid Tumor

In some embodiments, the cancer is a solid tumor.

In some embodiments, the solid tumor is a prostate cancer, a lung cancer, a non-small cell lung cancer (NSCLC), a liver cancer, a cervical cancer, a colon cancer, a breast cancer, an ovarian cancer, an endometrial cancer, a pancreatic cancer, a melanoma, an esophageal cancer, a gastric cancer, a stomach cancer, a renal carcinoma, a bladder cancer, a hepatocellular carcinoma, a renal cell carcinoma, an urothelial carcinoma, a head and neck cancer, a glioma, a glioblastoma, a colorectal cancer, a thyroid cancer, epithelial cancers, or adenocarcinomas.

In some embodiments, the prostate cancer is a relapsed prostate cancer. In some embodiments, the prostate cancer is a refractory prostate cancer. In some embodiments, the prostate cancer is a malignant prostate cancer. In some embodiments, the prostate cancer is a castration resistant prostate cancer.

### Definitions

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. In some embodiments, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. In some embodiments, BLAST and BLAST 2.0 algorithms and the default parameters are used.

A “comparison window,” as used herein, includes reference to a segment of any one of the number of contiguous positions selected from the group consisting of 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wi), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

As used herein, “administering” refers to local and systemic administration, e.g., including enteral, parenteral, pulmonary, and topical/transdermal administration. Routes of administration for pharmaceutical ingredients (e.g., vectors) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a pharmaceutical ingredient or composition to a mammal so that the pharmaceutical ingredient or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the pharmaceutical ingredient (e.g., vector) and/or analogs thereof and another active agent (e.g., multispecific antibody), refers to administration of the pharmaceutical ingredient and/or analogs and the active agent such that both can simultaneously achieve a physiological effect. The two agents, however, need not be administered together. In some embodiments, administration of one agent can precede administration of the other. Simultaneous physiological effect need not necessarily require presence of both agents in the circulation at the same time. However, in some embodiments, co-administering typically results in both agents being simultaneously present in the body (e.g., in the plasma) at a significant fraction (e.g., 20% or greater, e.g., 30% or 40% or greater, e.g., 50% or 60% or greater, e.g., 70% or 80% or 90% or greater) of their maximum serum concentration for any given dose.

The term “effective amount” or “pharmaceutically effective amount” refer to the amount and/or dosage, and/or dosage regime of one or more pharmaceutical ingredients (e.g., vectors) necessary to bring about the desired result.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In some embodiments, the reduction or elimination of one or more symptoms of pathology or disease can include, e.g., measurable and sustained decrease of tumor volume.

As used herein, the phrase “consisting essentially of” refers to the genera or species of active pharmaceutical agents recited in a method or composition, and further can include other agents that, on their own do not have substantial activity for the recited indication or purpose.

The terms “subject,” “individual,” and “patient” interchangeably refer to a mammal, preferably a human or a non-human primate, but also domesticated mammals (e.g., canine or feline), laboratory mammals, and agricultural mammals. In various embodiments, the subject can be a human (e.g., adult male, adult female, adolescent male, adolescent female, male child, female child).

The term “vector” as used herein refers a macromolecular complex capable of delivering a foreign nucleic acid molecule into a cell independent of another agent. As used herein, the term vector excludes naked nucleic acid molecules, such as plasmids, for naked nucleic acid molecules do not effectively transduce themselves into target cells independent of other factors (such as transfection reagents or electroporation). The vector can be a viral vector or non-viral vector. Viral vector includes retroviral vector and lentiviral vector. Non-viral vectors are limited to liposomes, nanoparticles, and other encapsulation systems for delivery of polynucleotides into cells.

As used herein, the term “expression cassette” refers to a DNA segment that is capable in an appropriate setting of driving the expression of a polynucleotide (a “transgene”) encoding a polypeptide (e.g., chimeric antigen receptor) that is incorporated in said expression cassette. When introduced into a host cell, an expression cassette inter alia is capable of directing the cell's machinery to transcribe the transgene into RNA, which is then usually further processed and finally translated into the polypeptide. The expression cassette can be comprised in a vector (e.g., viral vector). Generally, the term expression cassette excludes polynucleotide sequences 5′ to the 5′ ITR and 3′ to the 3′ ITR.

The term “derived” is used to indicate that the cells have been obtained from their biological source and grown or otherwise manipulated in vitro (e.g., cultured in a growth medium to expand the population and/or to produce a cell line).

The term “transduce” refers to introduction of a nucleic acid into a cell or host organism by way of a vector (e.g., a lentiviral vector). Introduction of a transgene into a cell by a viral vector can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell. Alternatively, the introduced transgene may exist in the recipient cell or host organism extra-chromosomally, or only transiently. A “transduced cell” is therefore a cell into which the transgene has been introduced by way of transduction. Thus, a “transduced” cell is a cell into which, a polynucleotide has been introduced.

The term “transduction efficiency” is an expression of the proportion of cells that express or transduce a transgene when a cell culture is contacted with vector particles. In some embodiments, the efficiency can be expressed as the number of cells expressing a transgene when a given number of cells are contacted with a given number of vector particles. In some embodiments, “Relative transduction efficiency” is the proportion of cells transduced by a given number of viral particles in one condition relative to the proportion of cells transduced by that same number of particles in another condition comprising a similar number of cells of the same cell type. Relative transduction efficiency is most often used to compare the effects of a modulator of transduction efficiency on cells and/or animals treated or not treated with that modulator.

All patents, patent publications, and other publications referenced and identified in the present specification are individually and expressly incorporated herein by reference in their entirety for all purposes.

### Further Numbered Embodiments—Section A

One set of the numbered embodiments of the present disclosure is provided as follows:
Clause 1. A method of transducing immune cells in a subject in need thereof, comprising:
a) administering a multispecific antibody to render immune cells in the subject more transducible; and
b) administering a vector, optionally a viral vector;
wherein the method transduces the immune cells.
Clause 2. The method of clause 1, wherein the immune cells are T cells.
Clause 3. The method of clause 1 or 2, wherein the vector is a lentiviral vector.
Clause 4. The method of any one of clauses 1 to 3, wherein the multispecific antibody comprises a T-cell antigen-specific binding domain.
Clause 5. The method of clause 4, wherein the T-cell antigen is CD3, CD4, CD8, or TCR.
Clause 6. The method of any one of clauses 1 to 5, wherein the multispecific antibody comprises a second antigen-specific binding domain.
Clause 7. The method of clause 6, wherein the second antigen is CD19.
Clause 8. The method of clause 6, wherein the second antigen is CD19, EpCAM, Her2/neu, EGFR, CD66e, CD33, EphA2, or MCSP.
Clause 9. The method of clause 6, wherein the second antigen is CD19, EpCAM, CD20, CD123, BCMA, B7-H3, CDE, or PSMA.
Clause 10. The method of clause 6, wherein the second antigen is a myeloid cell or dendritic cell antigen.
Clause 11. The method of clause 10, wherein the second antigen is CD33, DC-SIGN, CD11b, CD11c, or CD18.
Clause 12. The method of any one of clauses 1 to 11, wherein the multispecific antibody is a bispecific antibody.
Clause 13. The method of clause 12, wherein the bispecific antibody is a bispecific T-cell engager (BiTE).
Clause 14. The method of clause 13, wherein the BiTE is a CD19×CD3 BiTE.
Clause 15. The method of clause 14, wherein the CD19×CD3 BiTE is blinatumomab.
Clause 16. The method of any one of clauses 1 to 15, wherein the multispecific antibody activates the immune cells.
Clause 17. The method of any one of clauses 1 to 16, wherein the multispecific antibody increases activation of the immune cells compared to administration of a vehicle control.
Clause 18. The method of any one of clauses 1 to 18, wherein the multispecific antibody increases the number of immune cells in a lymph node of the subject.
Clause 19. The method of any one of clauses 1 to 18, wherein the multispecific antibody increases transduction of the immune cells compared to administration of the viral vector alone.
Clause 20. The method of any one of clauses 1 to 19, wherein the multispecific antibody enhances in vivo transduction of the immune cells by the viral vector.
Clause 21. The method of any one of clauses 1 to 20, wherein the multispecific antibody reduces the effective concentration (EC50) of the viral vector.
Clause 22. The method of any one of clauses 1 to 21, wherein the method achieves the same level of transduction of immune cells as a method comprising administering a higher concentration of the viral vector without administering the multispecific antibody.
Clause 23. The method of any one of clauses 1 to 22, wherein step a) and/or step b) comprises subcutaneous administration.
Clause 24. The method of any one of clauses 1 to 23, wherein step a) and/or step b) comprises intralymphatic administration.
Clause 25. The method of any one of clauses 1 to 24, wherein the viral vector comprises a polynucleotide encoding a T cell receptor or chimeric antigen receptor.
Clause 26. The method of clause 25, wherein the chimeric antigen receptor is an anti-CD19 chimeric antigen receptor.
Clause 27. The method of any one of clauses 1 to 26, wherein the viral vector comprises a polynucleotide encoding a cytokine receptor.
Clause 28. The method of clause 27, wherein the cytokine receptor is a drug-inducible cytokine receptor.
Clause 29. The method of any one of clauses 1 to 28, wherein the vector further comprises one or more transgenes.
Clause 30. The method of clause 29, wherein the viral vector comprises the transgene encoding a TGFβ dominant negative receptor.
Clause 31. The method of any one of clauses 3 to 30, wherein the lentiviral vector comprises one or more cell surface receptors that bind to a ligand on a target host cell, heterologous viral envelope glycoproteins, fusion glycoproteins, T-cell activation or co-stimulation molecules, ligands for CD19 or a functional fragment thereof, cytokines or cytokine-based transduction enhancers, and/or transmembrane proteins comprising a mitogenic domain and/or cytokine-based domain exposed on the surface and/or conjugated to the surface of the lentiviral vector.
Clause 32. The method of clause 31, wherein the one or more T-cell activation or co-stimulation molecules comprise one or more T-cell ligands.
Clause 33. The method of any one of clauses 3 to 32, wherein the lentiviral vector is pseudotyped with a Cocal virus envelop protein.
Clause 34. The method of any one of clauses 3 to 33, wherein the lentiviral vector is pseudotyped with a Nipah virus envelop protein.
Clause 35. The method of clause 34, wherein the Nipah envelop protein is engineered to bind EpCAM, CD4, or CD8.
Clause 36. The method of any one of clauses 1 to 35, wherein step a) or step b) comprises intravenous administration.
Clause 37. The method of clauses 36, wherein both step a) and step b) comprise intravenous administration.
Clause 38. The method of any one of clauses 1 to 37, where the multispecific antibody is administered at a dose of about 0.001 mg/kg to about 1 mg/kg.
Clause 39. The method of any one of clauses 1 to 38, wherein the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.
Clause 40. A method of transducing immune cells in a subject in need thereof, comprising:
a) administering a polynucleotide encoding a multispecific antibody to activate immune cells in the subject; and
b) administering a vector, optionally a viral vector;
wherein the method transduces the immune cells
Clause 41. The method of clause 40, wherein the polynucleotide encoding a multispecific antibody is an RNA.
Clause 42. The method of clause 40 or 41, wherein the immune cells are T cells.
Clause 43. The method of any one of clauses 40 to 42, wherein the vector is a lentiviral vector.
Clause 44. The method of any one of clauses 40 to 43, wherein the multispecific antibody comprises a T-cell antigen-specific binding domain.
Clause 45. The method of clause 44, wherein the T-cell antigen is CD3, CD4, CD8, or TCR.
Clause 46. The method of any one of clauses 40 to 45, wherein the multispecific antibody comprises a second antigen-specific binding domain.
Clause 47. The method of clause 46, wherein the second antigen is CD19.
Clause 48. The method of clause 46, wherein the second antigen is CD19, EpCAM, Her2/neu, EGFR, CD66e, CD33, EphA2, MCSP, CD22, CD79a, CD79b, or sIgM.
Clause 49. The method of clause 46, wherein the second antigen is CD19, EpCAM, CD20, CD123, BCMA, B7-H3, CDE, or PSMA.
Clause 50. The method of clause 46, wherein the second antigen is a lymph node antigen.
Clause 51. The method of any one of clauses 40 to 50, wherein the multispecific antibody is a trispecific antibody.
Clause 52. The method of any one of clauses 40 to 50, wherein the multispecific antibody is a bispecific antibody.
Clause 53. The method of clause 52, wherein the bispecific antibody is a bispecific T-cell engager (BiTE).
Clause 54. The method of clause 53, wherein the BiTE is a CD19×CD3 BiTE.
Clause 55. The method of clause 54, wherein the CD19×CD3 BiTE is blinatumomab.
Clause 56. The method of any one of clauses 40 to 55, wherein the multispecific antibody activates the immune cells.
Clause 57. The method of any one of clauses 40 to 56, wherein the multispecific antibody increases activation of the immune cells compared to administration of a vehicle control.
Clause 58. The method of any one of clauses 40 to 57, wherein the multispecific antibody increases the number of immune cells in a lymph node of the subject.
Clause 59. The method of any one of clauses 40 to 58, wherein the multispecific antibody increases transduction of the immune cells compared to administration of the viral vector alone.
Clause 60. The method of any one of clauses 40 to 59, wherein the multispecific antibody enhances in vivo transduction of the immune cells by the viral vector.
Clause 61. The method of any one of clauses 40 to 60, wherein the multispecific antibody reduces the effective concentration (EC50) of the viral vector.
Clause 62. The method of any one of clauses 40 to 61, wherein the method achieves the same level of transduction of immune cells as a method comprising administering a higher concentration of the viral vector without administering the multispecific antibody.
Clause 63. The method of any one of clauses 40 to 62, wherein step a) and/or step b) comprises subcutaneous administration.
Clause 64. The method of any one of clauses 40 to 63, wherein step a) and/or step b) comprises intralymphatic administration.
Clause 65. The method of any one of clauses 40 to 64, wherein step a) and/or step b) comprises intravenous administration.
Clause 66. The method of any one of clauses 40 to 65, wherein the viral vector comprises a polynucleotide encoding a T cell receptor or chimeric antigen receptor.
Clause 67. The method of clause 66, wherein the chimeric antigen receptor is an anti-CD19 chimeric antigen receptor.
Clause 68. The method of any one of clauses 40 to 67, wherein the viral vector comprises a polynucleotide encoding a cytokine receptor.
Clause 69. The method of clause 68, wherein the cytokine receptor is a drug-inducible cytokine receptor.
Clause 70. The method of any one of clauses 43 to 69, wherein the lentiviral vector comprises one or more cell surface receptors that bind to a ligand on a target host cell, heterologous viral envelope glycoproteins, fusion glycoproteins, T-cell activation or co-stimulation molecules, ligands for CD19 or a functional fragment thereof, cytokines or cytokine-based transduction enhancers, and/or transmembrane proteins comprising a mitogenic domain and/or cytokine-based domain exposed on the surface and/or conjugated to the surface of the lentiviral vector.
Clause 71. The method of clause 70, wherein the one or more T-cell activation or co-stimulation molecules comprise one or more T-cell ligands.
Clause 72. The method of any one of clauses 43 to 70, wherein the vector further comprises one or more transgenes.
Clause 73. The method of clause 72, wherein the viral vector comprises the transgene encoding a TGFβ dominant negative receptor.
Clause 74. The method of any one of clauses 43 to 73, wherein the lentiviral vector is pseudotyped with a Cocal virus envelop protein.
Clause 75. The method of any one of clauses 43 to 74, wherein the lentiviral vector is pseudotyped with a Nipah virus envelop protein.
Clause 76. The method of clause 75, wherein the Nipah envelop protein is engineered to bind EpCAM, CD4, or CD8.
Clause 77. The method of any one of clauses 43 to 77, wherein the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.
Clause 78. A combination therapy for use in transducing immune cells in vivo, comprising a multispecific antibody and a vector, optionally a viral vector.
Clause 79. A pharmaceutical composition, comprising a multispecific antibody and a vector, optionally a viral vector.
Clause 80. A kit comprising 1) a multispecific antibody and 2) a vector, optionally a viral vector.
Clause 81. A kit comprising 1) a polynucleotide encoding a multispecific antibody and 2) a vector, optionally a viral vector.
Clause 82. The kit of clause 80 or 81, for use in:
a) transducing immune cells in a subject in need thereof; and/or
b) treating a disease or disorder in a subject in need thereof.
Clause 83. A method of treating a disease or disorder in a subject in need thereof, comprising:
a) administering a multispecific antibody to activate immune cells in the subject; and
b) before, after and concurrently with step a), administering a vector, optionally a viral vector.
Clause 84. The method of clause 83, wherein the method transduces the immune cells.
Clause 85. The method of clause 83 or 84, wherein the disease or disorder is a cancer.
Clause 86. The method of clause 83 or 84, wherein the disease or disorder is a hematological malignancy.
Clause 87. The method of clause 86, wherein the hematological malignancy is B cell lymphoma.
Clause 88. The method of any one of clauses 83 to 87, wherein the method treats the disease or disorder faster than administering the multispecific antibody alone and/or the vector alone.
Clause 89. The method of any one of clauses 83 to 88, wherein the method results in a better treatment outcome of the disease or disorder than administering the multispecific antibody alone and/or the vector alone.
Clause 90. The method of any one of clauses 83 to 89, wherein the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.
Clause 91. The method of clause 87 or 90, wherein the method results in faster depletion of malignant B cells in the subject than administering the multispecific antibody alone and/or the vector alone.
Clause 92. The method of any one of clauses 87 or 90-91, wherein the method results in lower number of residual malignant B cells and/or lower recurrence rate of the B cell lymphoma in the subject than administering the multispecific antibody alone and/or the vector alone.

### Further Numbered Embodiments—Section B

Another set of further numbered embodiments of the present disclosure is provided as the following clauses:
Clause 1. A vector for use in a method of treatment, wherein the vector comprises a polynucleotide; and wherein the method comprises:
(a) administering a multispecific antibody to render an immune cell in the subject more transducible; and
(b) administering the vector to the subject in order to transduce the immune cell, wherein the transduction comprises the delivery of the polynucleotide to the cell.
Clause 2. A multispecific antibody for use in a method of treatment, wherein
the method comprises:
(a) administering the multispecific antibody to render an immune cell in the subject more transducible; and
(b) administering a vector comprising a polynucleotide to the subject in order to transduce the immune cell, wherein the transduction comprises the delivery of the polynucleotide to the cell.
Clause 3. A polynucleotide for use in a method of treatment, wherein
the method comprises:
(a) administering a multispecific antibody to render an immune cell in the subject more transducible; and
(b) administering a vector comprising the polynucleotide to the subject in order to transduce the immune cell, wherein the transduction comprises the delivery of the polynucleotide to the cell.
Clause 4. The vector for use according to Clause 1, the multispecific antibody for use according to Clause 2, or the polynucleotide for use according to Clause 3, wherein the immune cell is a T cell.
Clause 5. The vector for use according to Clause 1 or clause 4, the multispecific antibody for use according to Clause 2 or clause 4, or the polynucleotide for use according to Clause 3 or clause 4, wherein the multispecific antibody activates the immune cell; optionally wherein the activation leads to an increase in CD71 expression.
Clause 6. The vector for use according to any one of clauses 1 and 4-5, the multispecific antibody for use according to any one of clauses 2 and 4-5, or the polynucleotide for use according to any one of clauses 3-5, wherein the multispecific antibody increases activation of the immune cell compared to administration of a vehicle control; optionally wherein the activation leads to an increase in CD71 expression.
Clause 7. The vector for use according to any one of clauses 1 and 4-6, the multispecific antibody for use according to any one of clauses 2 and 4-6, or the polynucleotide for use according to any one of clauses 3-6, wherein the multispecific antibody:
(i) increases the number of immune cells in a lymph node of the subject; and/or
(ii) increases transduction of the immune cell compared to administration of the vector alone; and/or
(iii) enhances in vivo transduction of the immune cell by the vector; and/or
(iv) reduces the effective concentration (EC50) of the vector.
Clause 8. The vector for use according to any one of clauses 1 and 4-7, the multispecific antibody for use according to any one of clauses 2 and 4-7, or the polynucleotide for use according to any one of clauses 3-7, wherein the method achieves the same level of transduction of immune cells as a method comprising administering a higher concentration of the vector without administering the multispecific antibody.
Clause 9. The vector for use according to any one of clauses 1 and 4-8, the multispecific antibody for use according to any one of clauses 2 and 4-8, or the polynucleotide for use according to any one of clauses 3-8, wherein step a) and/or step b) comprise:
(i) subcutaneous administration; or
(ii) intralymphatic administration; or
(iii) intravenous administration; optionally wherein both step a) and step b) comprise intravenous administration.
Clause 10. The vector for use according to any one of clauses 1 and 4-9, the multispecific antibody for use according to any one of clauses 2 and 4-9, or the polynucleotide for use according to any one of clauses 3-9, wherein the multispecific antibody is administered at a dose of about 0.001 mg/kg to about 1 mg/kg.
Clause 11. The vector for use according to any one of clauses 1 and 4-10, the multispecific antibody for use according to any one of clauses 2 and 4-10, or the polynucleotide for use according to any one of clauses 3-10, wherein step b) occurs before, after, and/or concurrently with step a).
Clause 12. The vector for use according to any one of clauses 1 and 4-11, the multispecific antibody for use according to any one of clauses 2 and 4-11, or the polynucleotide for use according to any one of clauses 3-11, wherein the method is for the treatment of a cancer.
Clause 13. The vector for use according to any one of clauses 1 and 4-12, the multispecific antibody for use according to any one of clauses 2 and 4-12, or the polynucleotide for use according to any one of clauses 3-12, wherein the method is for the treatment of a hematological malignancy; optionally wherein the hematological malignancy is B cell lymphoma.
Clause 14. The vector for use according to any one of clauses 1 and 4-13, the multispecific antibody for use according to any one of clauses 2 and 4-13, or the polynucleotide for use according to any one of clauses 3-13, wherein the method is for the treatment of a disease or disorder, and wherein
the method treats the disease or disorder faster than administering the multispecific antibody alone and/or the vector alone; or
   the method results in a better treatment outcome of the disease or disorder than administering the multispecific antibody alone and/or the vector alone.
Clause 15. The vector, multispecific antibody, or the polynucleotide for use according to Clause 13, wherein the method results in faster depletion of malignant B cells in the subject than administering the multispecific antibody alone and/or the vector alone.
Clause 16. The vector, multispecific antibody, or the polynucleotide for use according to Clause 13 or clause 15, wherein the method results in lower number of residual malignant B cells and/or lower recurrence rate of the B cell lymphoma in the subject than administering the multispecific antibody alone and/or the vector alone.
Clause 17. The vector for use according to any one of clauses 1 and 4-16, the multispecific antibody for use according to any one of clauses 2 and 4-16, or the polynucleotide for use according to any one of clauses 3-16, wherein the multispecific antibody is administered as a polynucleotide encoding the multispecific antibody.
Clause 18. A pharmaceutical composition, comprising a multispecific antibody and a vector.
Clause 19. A kit comprising 1) a multispecific antibody and 2) a vector.
Clause 20. A kit comprising 1) a polynucleotide encoding a multispecific antibody and 2) a vector.
Clause 21. The vector, multispecific antibody, or the polynucleotide for use according to Clause 17, or the kit according to Clause 20, wherein the polynucleotide encoding the multispecific antibody is an RNA.
Clause 22. The vector for use according to any one of clauses 1, 4-17, and 21, the multispecific antibody for use according to any one of clauses 2, 4-17, and 21, the polynucleotide for use according to any one of clauses 3-17 and 21, the pharmaceutical composition according to Clause 18, or the kit according to any one of clauses 19-21, wherein the vector is a viral vector; optionally a lentiviral vector.
Clause 23. The vector for use according to any one of clauses 1, 4-17, and 21-22, the multispecific antibody for use according to any one of clauses 2, 4-17, and 21-22, the polynucleotide for use according to any one of clauses 3-17 and 21-22, the pharmaceutical composition according to Clause 18 or clause 22, or the kit according to any one of clauses 19-22, wherein the multispecific antibody comprises a T-cell antigen-specific binding domain; optionally wherein the T-cell antigen is CD3, CD4, CD8, or TCR.
Clause 24. The vector for use according to any one of clauses 1, 4-17, and 21-23, the multispecific antibody for use according to any one of clauses 2, 4-17, and 21-23, the polynucleotide for use according to any one of clauses 3-17 and 21-23, the pharmaceutical composition according to any one of clauses 18 and 22-23, or the kit according to any one of clauses 19-23, wherein the multispecific antibody comprises a second antigen-specific binding domain; optionally wherein:
(i) the second antigen is CD19; or
(ii) the second antigen is CD19, EpCAM, Her2/neu, EGFR, CD66e, CD33, EphA2, or MCSP; or
(iii) the second antigen is CD19, EpCAM, Her2/neu, EGFR, CD66e, CD33, EphA2, MCSP, CD22, CD79a, CD79b, or sIgM
(iv) the second antigen is CD19, EpCAM, CD20, CD123, BCMA, B7-H3, CDE, or PSMA; or
(v) the second antigen is a lymph node antigen; or
(vi) the second antigen is a myeloid cell or dendritic cell antigen; optionally CD33, DC-SIGN, CD11b, CD11c, or CD18.
Clause 25. The vector for use according to any one of clauses 1, 4-17, and 21-24, the multispecific antibody for use according to any one of clauses 2, 4-17, and 21-24, the polynucleotide for use according to any one of clauses 3-17 and 21-24, the pharmaceutical composition according to any one of clauses 18 and 22-24, or the kit according to any one of clauses 19-24, wherein the multispecific antibody is a bispecific antibody or a trispecific antibody; optionally wherein the bispecific antibody is a bispecific T-cell engager (BiTE); optionally wherein the BiTE is a CD19×CD3 BiTE; and optionally wherein the CD19×CD3 BiTE is blinatumomab.
Clause 26. The vector for use according to any one of clauses 1, 4-17, and 21-25, the multispecific antibody for use according to any one of clauses 2, 4-17, and 21-25, the polynucleotide for use according to any one of clauses 3-17 and 21-25, the pharmaceutical composition according to any one of clauses 18 and 22-25, or the kit according to any one of clauses 19-25, wherein the polynucleotide that is delivered to the cell encodes at least one therapeutic polypeptide.
Clause 27. The vector, multispecific antibody, or polynucleotide for use according to Clause 26, or the pharmaceutical composition or kit according to Clause 26, wherein the at least one therapeutic polypeptide comprises a T cell receptor or a chimeric antigen receptor; and optionally wherein the T cell receptor or chimeric antigen receptor target an antigen associated with a cancer or a hematological malignancy.
Clause 28. The vector, multispecific antibody, or polynucleotide for use according to Clause 27, or the pharmaceutical composition or kit according to Clause 27, wherein the chimeric antigen receptor is an anti-CD19 chimeric antigen receptor.
Clause 29. The vector, multispecific antibody, or polynucleotide for use according to Clause 26, or the pharmaceutical composition or kit according to Clause 26, wherein the at least one therapeutic polypeptide comprises a cytokine receptor; optionally wherein the cytokine receptor is a drug-inducible cytokine receptor.
Clause 30. The vector for use according to any one of clauses 1, 4-17, and 21-29, the multispecific antibody for use according to any one of clauses 2, 4-17, and 21-29, the polynucleotide for use according to any one of clauses 3-17 and 21-29, the pharmaceutical composition according to any one of clauses 18 and 22-29, or the kit according to any one of clauses 19-29, wherein the vector further comprises one or more transgenes; optionally wherein the one or more transgenes comprise a transgene encoding a TGFβ dominant negative receptor.
Clause 31. The vector for use according to any one of clauses 1, 4-17, and 21-30, the multispecific antibody for use according to any one of clauses 2, 4-17, and 21-30, the polynucleotide for use according to any one of clauses 3-17 and 21-30, the pharmaceutical composition according to any one of clauses 18 and 22-30, or the kit according to any one of clauses 19-30, wherein the vector comprises one or more cell surface receptors that bind to a ligand on a target host cell, heterologous viral envelope glycoproteins, fusion glycoproteins, T-cell activation or co-stimulation molecules, ligands for CD19 or a functional fragment thereof, cytokines or cytokine-based transduction enhancers, and/or transmembrane proteins comprising a mitogenic domain and/or cytokine-based domain exposed on the surface and/or conjugated to the surface of the vector; optionally wherein the one or more T-cell activation or co-stimulation molecules comprise one or more T-cell ligands.
Clause 32. The vector for use according to any one of clauses 1, 4-17, and 21-31, the multispecific antibody for use according to any one of clauses 2, 4-17, and 21-31, the polynucleotide for use according to any one of clauses 3-17 and 21-31, the pharmaceutical composition according to any one of clauses 18 and 22-31, or the kit according to any one of clauses 19-31, wherein the vector is a lentiviral vector, and wherein the lentiviral vector is pseudotyped with a Cocal virus envelop protein and/or a Nipah virus envelop protein; optionally wherein the Nipah envelop protein is engineered to bind EpCAM, CD4, or CD8.
Clause 33. The vector for use according to any one of clauses 1, 4-17, and 21-32, the multispecific antibody for use according to any one of clauses 2, 4-17, and 21-32, the polynucleotide for use according to any one of clauses 3-17 and 21-32, the pharmaceutical composition according to any one of clauses 18 and 22-32, or the kit according to any one of clauses 19-32, wherein the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.
Clause 34. The kit according to any one of clauses 19-33, for use in a method of treatment.

### EXAMPLES

### Example 1: Blinatumomab Enhances Transduction of T Cells by Lentiviral Vector

This examples relates to use of a subcutaneously, intralymphatically, or intratumorily injected CD19×CD3 bispecific antibody (blinatumomab) to activate T-cells in a lymph node and thereby increase transduction by a surface engineered lentiviral vector.

Lentiviral Vector

VivoVec

Lentiviral vector (VivoVec) was generated using a modified third-generation packaging system. To generate lentiviral particles, an envelope plasmid encoding a 2A-linked polycistronic expression construct (CD86-2A-anti-CD3scFv-2A-CD137L-2A-COCVG) was co-transfected into 293 cells with a transfer plasmid encoding an anti-CD19 chimeric antigen receptor operatively linked to a constitutive CMV promoter or MND promoter, flanked by 5′ and 3′ long terminal repeats; and two packaging plasmids encoding gag and pol, and rev genes, respectively.

Expression of CD86, a transmembrane-fused anti-CD3 single-chain variable fragment, CD137L, and the G protein of the Cocal virus (COCVG) from the envelope plasmid results in a lentiviral vector surface engineered to be specific for CD3-expressing cells (anti-CD3); to stimulate T cells (CD86 and CD137L); and to transduce T cells (pseudotyping with Cocal G protein). The genome of the lentiviral vector is the RNA transcribed by the Pol protein from the transfer plasmid. Gag mediates virus packaging of the G protein, Gag, and Rev proteins along with the RNA genome.

VSV Particles

Lentiviral vector (VSV particle) was generated using a modified third-generation packaging system. To generate lentiviral particles, an envelope plasmid encoding the G protein of vesicular stomatitis virus (VSV) was co-transfected into 293 cells with a transfer plasmid encoding an anti-CD19 chimeric antigen receptor operatively linked to a constitutive CMV promoter or MND promoter, flanked by 5′ and 3′ long terminal repeats; and two packaging plasmids encoding gag and pol, and rev genes, respectively.

Expression of the G protein of the VSV virus (VSVG) from the envelope plasmid results in a lentiviral vector pseudotyped with VSV G protein. The genome of the lentiviral vector is the RNA transcribed by the Pol protein from the transfer plasmid. Gag mediates virus packaging of the G protein, Gag, and Rev proteins along with the RNA genome.

Multispecific (Bispecific) Antibody

Blinatumomab is a bi-specific antibody of the “BiTE” class that is clinically approved to treat a CD19-expressing hematologic malignancy, B-cell precursor acute lymphoblastic leukemia (ALL). Blinatumomab's therapeutic mechanism of action involves the creation of an “immune synapse” between a T-cell and a tumor B-cell that induces the T-cell to kill the tumor B-cell. Here blinatumomab's biochemical activity is applied for an alternative purpose—administration to patients (with or without hematologic malignancies) for the purpose of causing normal B-cells to engage with T-cells in vivo for the purpose of rendering a T-cell susceptible to lentiviral transduction.

Demonstration of Increased Transduction In Vitro

Purified human primary T cells were thawed and resuspended in 3 mL of culture media (RPMI-1640 plus 10% fetal bovine serum). T cells were used either alone or in 50:50 ratio with B cells. Treatment tested were: vehicle control; blinatumomab alone; blinatumomab plus lentiviral particles (VivoVec); control VSV-pseudotyped lentiviral particles; and blinatumomab plus control VSV-pseudotyped lentiviral particles.

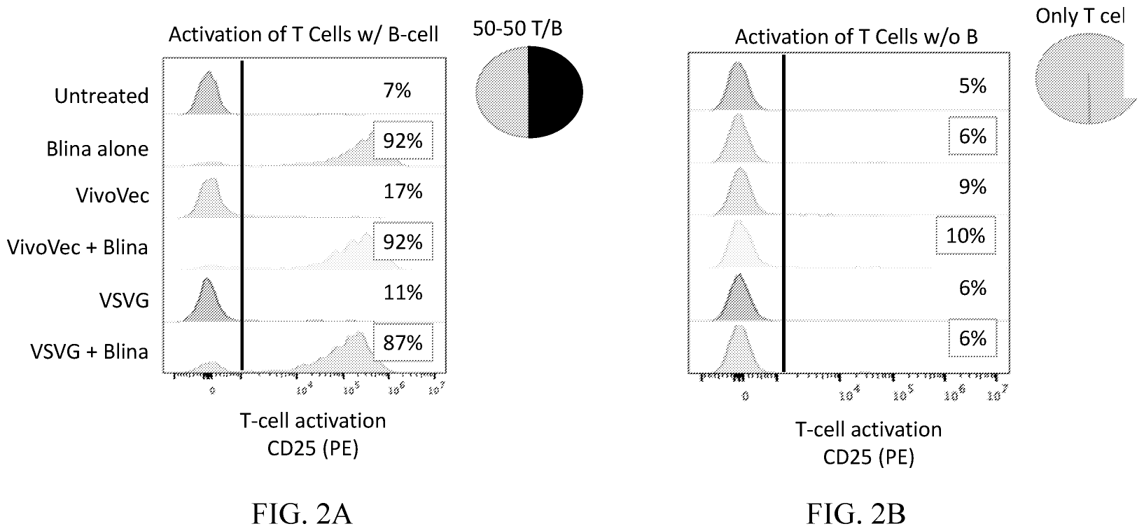

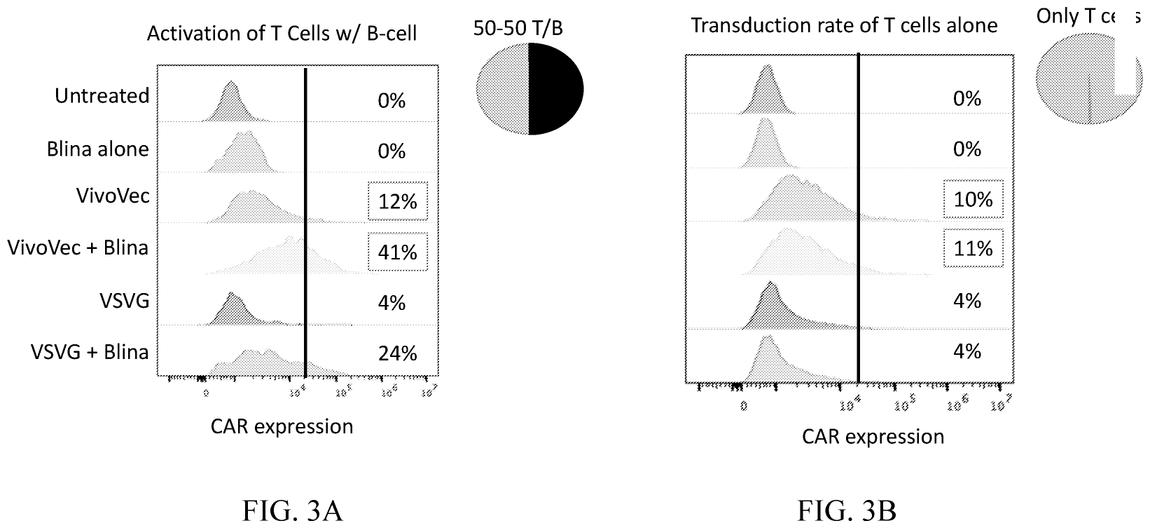

At three days after initiation of culture, cells were collected and analyzed by flow cytometry for expression of T-cell activation surface marker CD25 (FIG. 2A and FIG. 2B) and for expression of the vector-delivered anti-CD19 chimeric antigen receptor (FIG. 3A and FIG. 3B). Blinatumomab (“Blina”) increased the concentration of CD25+ activated T cells from about 7-17% to about 87-92% (FIG. 2A). The effect was dependent on presence of B cells in the culture, as without B cells no increase in activation was observed in blinatumomab-treated samples compared to samples not treated with blinatumomab (FIG. 2B). Blinatumomab (“Blina”) increased the percentage of T cells transduced by the VivoVec vector from about 12% to about 41%; it also increased transduction by VSV particles (“VSVG”) from about 4% to about 24% (FIG. 3A). The effect was dependent on presence of B cells in the culture, as without B cells VivoVec transduced about 10-11% of T cells and VSV particles transduced about 4% of T cells (FIG. 3B).

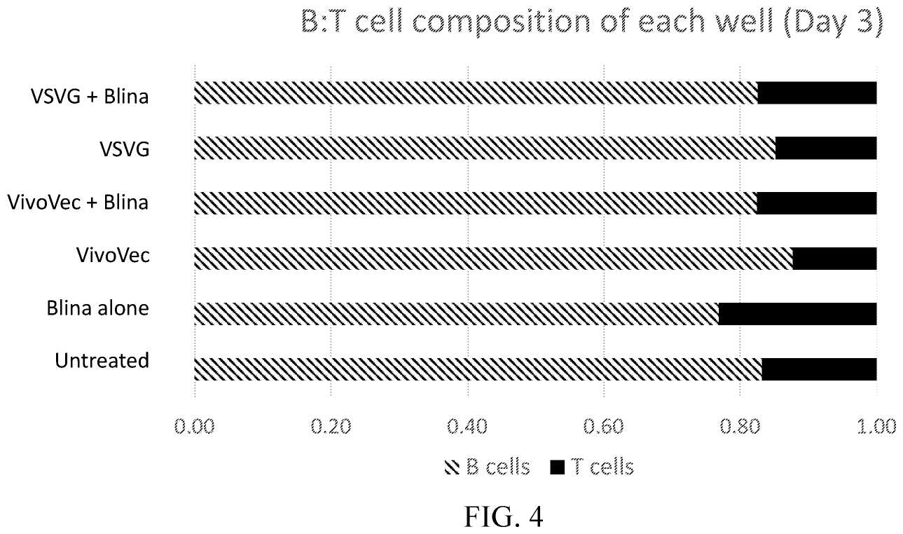

In 50:50 B:T cell samples, the ratio of B- to T-cells in the culture was assessed. B cell expansion resulted in about 80:20 final ratio. The ratio is similar for all test treatments, demonstrating that blinatumomab can be used for T cell activation at concentrations that do not lead to rapid B-cell killing (FIG. 4).

Demonstration of Increased Transduction In Vivo

Subjects (mice, primates, or humans) are administered blinatumomab at a sub-clinical dose (in humans, about 1 mg to about 10 mg in a single injection on day 1) prior to or concurrently with a lentiviral vector such as VivoVec. Lymph node biopsy is used to isolate T cells, which are assayed for expression of a transgene (e.g. anti-CD19 chimeric antigen receptor). Subjects having a B-cell malignancies are treated with VivoVec in combination with blinatumomab. Reduced disease progress, measure by B cell load and tumor size, is observed.

### Example 2: Blinotumomab-Mediated In Vivo Transduction of T Cells by Lentiviral Vector

We assessed in vivo transduction of T cells by a lentiviral vector encoding anti-CD19 CARs in CD34 humanized NSG mice, using Blinatumomab as a tool to activate T cells in vivo to facilitate transduction. The main questions addressed in this study were 1) does Blinatumomab promote CAR generation, 2) what dose of Blinatumomab is necessary for CAR T cell generation, and 3) can we detect CAR+ cells and does it correlate with B cell depletion.

Study Design

Virus Preparation and QC Data

Virus payload: U4367EA110_5, pRRL-MND-Human-Frb-CD19_CAR-TGFBdn-VTw. Cocal-pseudotyped lentivirus particles carrying an anti-CD19-CAR payload that also expressed FRB and a TGFβ dominant negative receptor (CD19CAR-TGFβ) payload were manufactured at Fred Hutchinson following a protocol similar to the one used in Example 1. Endotoxin activity was 2.1 EU/mL as measured by Chromogenic Endotoxin Quant Kit (Cat #A39552S). Culture was negative for mycoplasma as determined by Lonza MycoAlert Mycoplasma Detection kit.

Animal Study Protocol

Animal study was conducted at Lumigenics LLC using CD34+ humanized mice (Jackson Laboratories). HuNSG mice at 18-26 weeks post CD34+ HSC implantation were used for the study purpose. Mice were allowed to acclimate 1 week after arrival. Blood was collected on study day−2 to assess engraftment. Mice were distributed among study arms to ensure equivalent human T cell characteristics between groups.

Blood was collected weekly during the length of the study, starting on day−2, into EDTA-coated tubes. At least 70 μL was collected per blood draw. Samples were mixed by inversion and shipped to Umoja overnight on cold packs. Body weight was measured twice a week for the length of the study. Animals showing weight loss of more than 20% of initial body weight would be euthanized and recorded as “conditional death”.

Mice in the Blinatumomab treatment arms were treated intravenously (IV) on days 1, 2, and 4 of the study. Mice in the appropriate arms were treated with lentivirus via IV injection on study day 4.

Upon study completion on day 52, blood, a small section of spleen, and a femur was shipped to Umoja Biopharma on cold packs for analysis by flow cytometry.

Study endpoints include: 1) CAR-T cell transduction and expansion by flow cytometry, 2) CAR-T cell phenotype by flow cytometry, 3) B cell depletion, and 4) Toxicity, survival.

Table 2 below outlines the timeline of the study.

TABLE 2

| | | | | Lenti- | | | |
|---|---|---|---|---|---|---|---|
| | | | | virus | | | |
| | | | | (Day 4 | Lenti- | | |
| | | Blinatumomab | Blinatumomab | afternoon | virus | | Sample |
| Group | n | (Day 1, 2, 4) | volume | IV) | Volume | Raji cells | collection |
| 1 (Lenti only) | 4 | N/A | 5 ml/kg | 25 | 200 μL | 2.0E5 Day 24 | Day −2, 5, 12, |
| | | | | million | | 5.0E5 Day 39 | 19, 26, 33, 40, |
| | | | | TU | | | 47, 52 |
| 2a (Low Blin) | 3 | 0.004 mg/kg | 5 ml/kg | N/A | N/A | 2.0E5 Day 24 | Day −2, 5, 12, |
| | | IV | | | | 5.0E5 Day 39 | 19, 26, 33, 40, |
| | | | | | | | 47, 52 |
| 2b (High Blin) | 3 | 0.04 mg/kg | 5 ml/kg | N/A | N/A | 2.0E5 Day 24 | Day −2, 5, 12, |
| | | IV | | | | 5.0E5 Day 39 | 19, 26, 33, 40, |
| | | | | | | | 47, 52 |
| 3 (Low Blin | 5 | 0.004 mg/kg | 5 ml/kg | 25 | 200 μL | 2.0E5 Day 24 | Day −2, 5, 12, |
| and Lenti) | | IV | | million | | 5.0E5 Day 39 | 19, 26, 33, 40, |
| | | | | TU | | | 47, 52 |
| 4 (High Blin | 5 | 0.04 mg/kg | 5 ml/kg | 25 | 200 μL | 2.0E5 Day 24 | Day −2, 5, 12, |
| and Lenti) | | IV | | million | | 5.0E5 Day 39 | 19, 26, 33, 40, |
| | | | | TU | | | 47, 52 |

Results

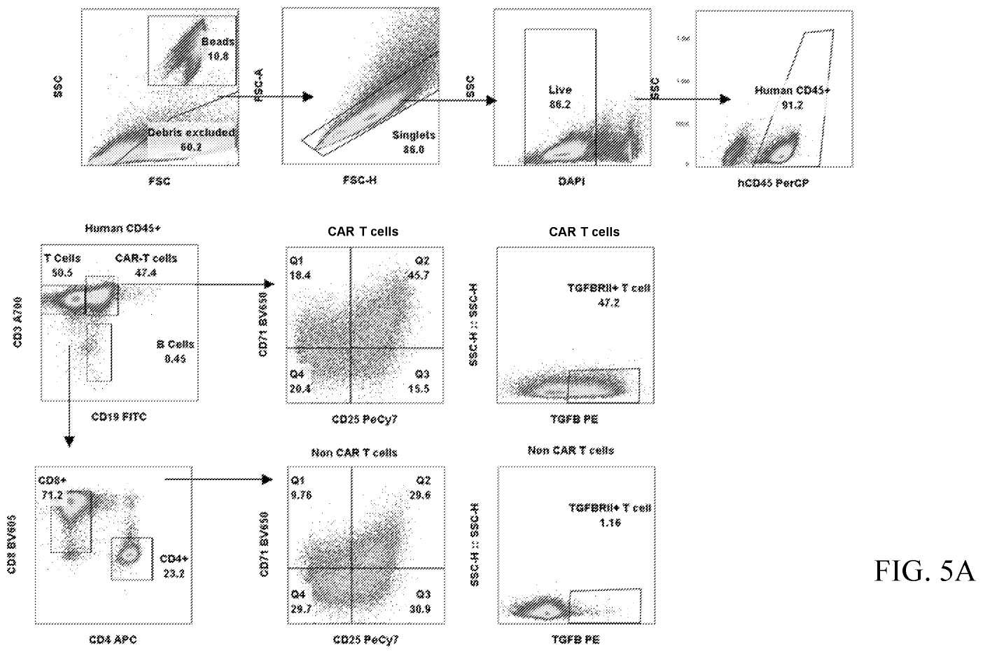

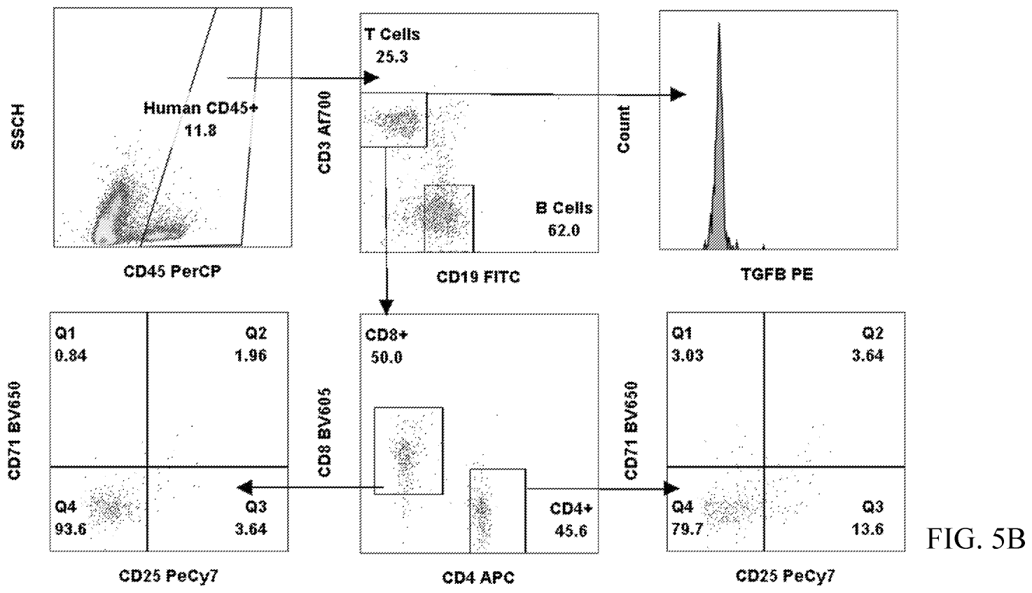

Validation of Flow Cytometry Panel: we validated our flow cytometry panel against anti CD19CAR-TGFβ T cells generated and maintained in culture (FIG. 5A) and in humanized mice (FIG. 5B). CAR T cells generated ex vivo were used as positive controls at each sample collection day. In FIG. 5A, ex vivo manufactured CAR-TGFβ T cells were used to validate CAR T cell detection via detection of TGFβ double negative receptor. All populations are gated off debris excluded/singlets/live/human CD45. CAR T cells were defined as CD3+ and FITC+. Non CAR T cells were defined as CD3+ and FITC−. The non CAR T cell population was used as a negative control to define positive staining against the TGFβ double negative receptor. FIG. 5B shows the gating scheme for identifying CD3+ T cells that are either CD4+ or CD8+ and that express CD25 or CD71 activation markers.

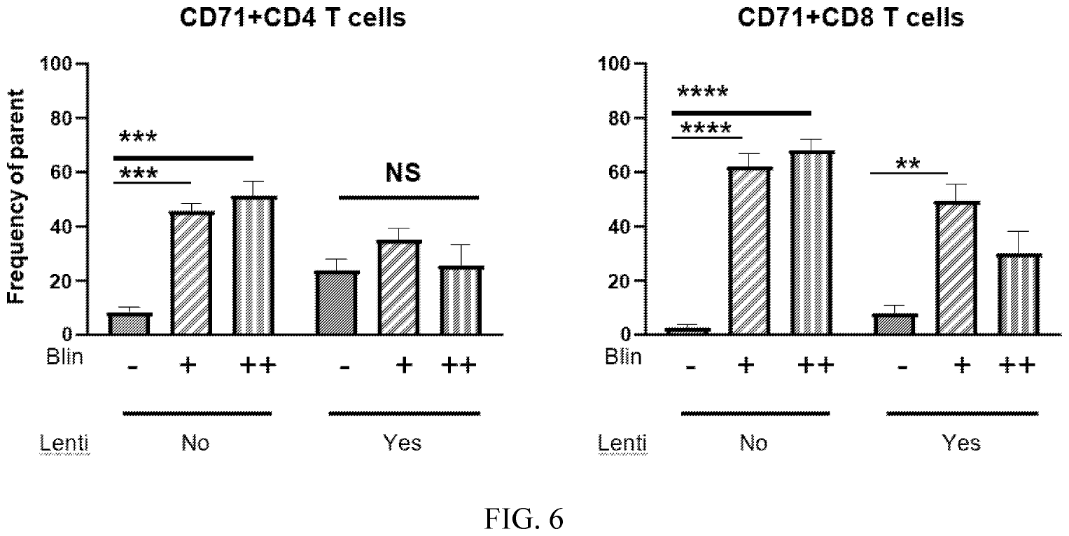

Blinatumomab administration activated T cells as measured by CD71 expression on day 5 post injection in both CD4 and CD8 T cells, as shown in FIG. 6 (“+” indicates “Low blin” groups and “++” indicates “High blin” groups; “**”, “***” and “****” indicate p values of <0.01, <0.001, <0.0001, respectively). CD25 was included in the flow panel but not used as an in vivo activation marker because very low expression of CD25 was observed in all groups at all timepoints.

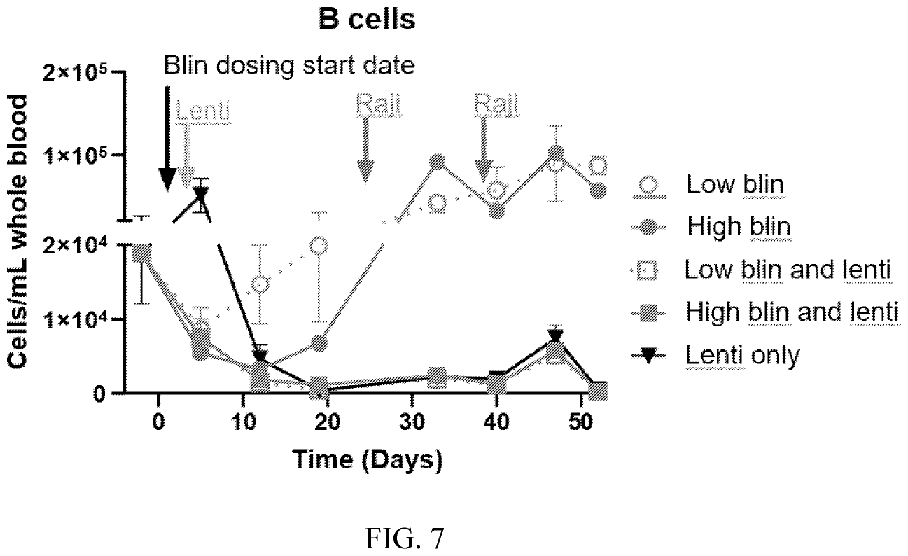

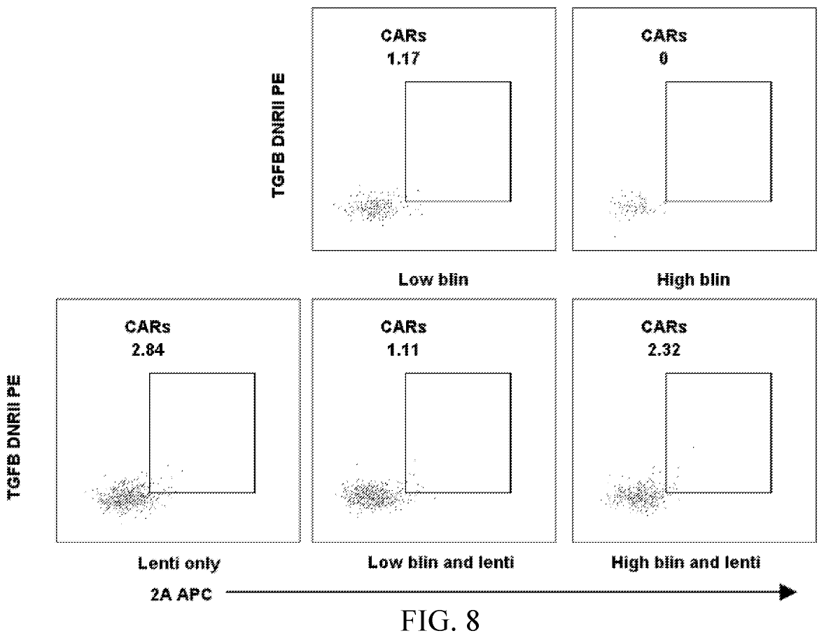

We also measured circulating B cells in mice treated with 0.004 mg/kg Blinotumomab (low blin), 0.04 mg/kg (high blin), with or without CD19CAR-TGFβ cocal lentivirus treatment (FIG. 7). Blinatumomab only treatment groups displayed immediate B cell depletion around day 5 but the number of B cells increased afterwards. On the other hand, CD19CAR-TGFβ cocal lentivirus treatment resulted in profound and prolonged B cell depletion, regardless of Blinatumomab administration. Mice in the lentivirus-only treated study arms were depleted of B cells beginning around day 12, whereas B cell depletion began around day 5 in mice in the lentivirus+Blinatumomab treated study arms. All mice in lentivirus treated arms had few, or no, circulating B cells through study days 12-52 (FIG. 7). We did not observe a significant population of CAR T cells during the length of the study in any group, using the Blinatumomab-only arms as the negative gating controls (FIG. 8).

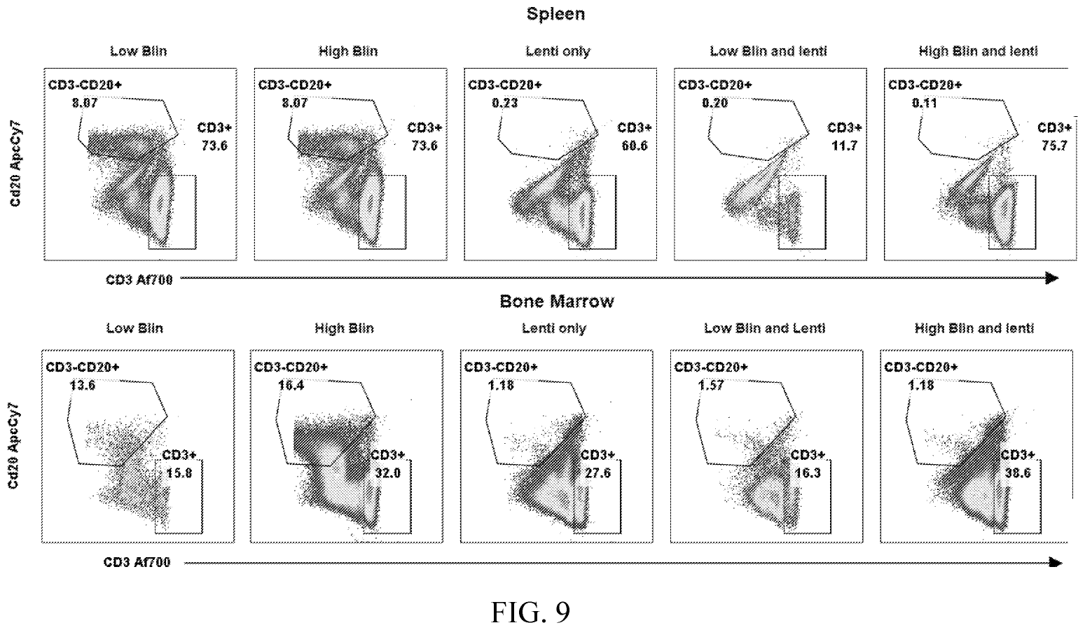

Complete B cell eradication was observed in the bone marrow and spleen of lentivirus-treated mice on study day 52 (FIG. 9). Due to rapid B cell depletion, we speculated that a transient CAR+ population was present at a level below our detection threshold.

In summary, intravenous administration of Cocal-enveloped lentivirus with a CD19CAR-TGFβ payload was sufficient to induce profound and prolonged B cell depletion in CD34 humanized mice. Blinatumomab administration accelerated B cell depletion. Upon study termination, no B cells were detected in the spleen or bone marrow of lentivirus-treated arms. In contrast, mice treated with both dosing levels of Blinatumomab, but no lentivirus, recovered circulating B cell populations after transient depletion, and had readily detectable B cell populations in the bone marrow and spleen (FIG. 9). These results are consistent with the predicted activity of in vivo anti CD19 CAR T cell generation.

### Example 3: Co-Administration of Blinotumomab and Lentiviral Vector

This examples relates to co-administration of a CD19×CD3 bispecific antibody (blinatumomab) and a surface engineered lentiviral vector comprising a transgene encoding an anti-CD19 CAR.

CD34+ humanized mice are co-administered with blinatumomab and the lentiviral vector. In the corresponding control groups, mice are administered with either blinatumomab alone, the lentiviral vector alone, or a mock solution. The administration may be performed subcutaneously, intralymphatically, and/or intratumorily. Blood and tissue samples (e.g., liver, lungs, spleen, bone marrow) are collected for analysis of the following factors: 1) CAR-T cell transduction and expansion analyzed by flow cytometry, 2) CAR-T cell phenotype analyzed by flow cytometry, 3) number of B cells, and 4) Toxicity, survival. The results of the co-administration group are compared with the control groups.

What is claimed is:

1. A method of transducing immune cells in a subject in need thereof, comprising:
a) administering a multispecific antibody to render immune cells in the subject more transducible; and
b) administering a vector, optionally a viral vector;
wherein the method transduces the immune cells.

2. The method of claim 1, wherein the immune cells are T cells.

3. The method of claim 1, wherein the vector is a lentiviral vector.

4. The method of claim 2, wherein the multispecific antibody comprises a T-cell antigen-specific binding domain.

5. The method of claim 4, wherein the T-cell antigen is CD3, CD4, CD8, or TCR.

6. The method of claim 4, wherein the multispecific antibody comprises a second antigen-specific binding domain.

7. The method of claim 6, wherein the second antigen is CD19.

8. The method of claim 6, wherein the second antigen is CD19, EpCAM, Her2/neu, EGFR, CD66e, CD33, EphA2, or MCSP.

9. The method of claim 6, wherein the second antigen is CD19, EpCAM, CD20, CD123, BCMA, B7-H3, CDE, or PSMA.

10. The method of claim 6, wherein the second antigen is a myeloid cell or dendritic cell antigen.

11. The method of claim 10, wherein the second antigen is CD33, DC-SIGN, CD11b, CD11c, or CD18.

12. The method of claim 1, wherein the multispecific antibody is a bispecific antibody.

13. The method of claim 12, wherein the bispecific antibody is a bispecific T-cell engager (BiTE).

14. The method of claim 13, wherein the BiTE is a CD19×CD3 BiTE.

15. The method of claim 14, wherein the CD19×CD3 BiTE is blinatumomab.

16. The method of any one of claims 1 to 15, wherein the multispecific antibody activates the immune cells.

17. The method of claim 16, wherein the multispecific antibody increases activation of the immune cells compared to administration of a vehicle control.

18. The method of any one of claims 1 to 15, wherein the multispecific antibody increases the number of immune cells in a lymph node of the subject.

19. The method of any one of claims 1 to 15, wherein the multispecific antibody increases transduction of the immune cells compared to administration of the viral vector alone.

20. The method of any one of claims 1 to 15, wherein the multispecific antibody enhances in vivo transduction of the immune cells by the viral vector.

21. The method of claim 20, wherein the multispecific antibody reduces the effective concentration (EC₅₀) of the viral vector.

22. The method of any one of claims 1 to 15, wherein the method achieves the same level of transduction of immune cells as a method comprising administering a higher concentration of the viral vector without administering the multispecific antibody.

23. The method of claim 1, wherein step a) and/or step b) comprises subcutaneous administration.

24. The method of claim 1, wherein step a) and/or step b) comprises intralymphatic administration.

25. The method of claim 2, wherein the vector is a viral vector comprising a polynucleotide encoding a T cell receptor or chimeric antigen receptor.

26. The method of claim 25, wherein the chimeric antigen receptor is an anti-CD19 chimeric antigen receptor.

27. The method of claim 2, wherein the vector is a viral vector comprising a polynucleotide encoding a cytokine receptor.

28. The method of claim 27, wherein the cytokine receptor is a drug-inducible cytokine receptor.

29. The method of claim 25, wherein the vector further comprises one or more transgenes.

30. The method of claim 29, wherein the viral vector comprises the transgene encoding a TGFβ dominant negative receptor.

31. The method of claim 3, wherein the lentiviral vector comprises one or more cell surface receptors that bind to a ligand on a target host cell, heterologous viral envelope glycoproteins, fusion glycoproteins, T-cell activation or co-stimulation molecules, ligands for CD19 or a functional fragment thereof, cytokines or cytokine-based transduction enhancers, and/or transmembrane proteins comprising a mitogenic domain and/or cytokine-based domain exposed on the surface and/or conjugated to the surface of the lentiviral vector.

32. The method of claim 31, wherein the one or more T-cell activation or co-stimulation molecules comprise one or more T-cell ligands.

33. The method of claim 31, wherein the lentiviral vector is pseudotyped with a Cocal virus envelop protein.

34. The method of claim 31, wherein the lentiviral vector is pseudotyped with a Nipah virus envelop protein.

35. The method of claim 34, wherein the Nipah envelop protein is engineered to bind EpCAM, CD4, or CD8.

36. The method of claim 1, wherein step a) or step b) comprises intravenous administration.

37. The method of claim 36, wherein both step a) and step b) comprise intravenous administration.

38. The method of claim 36 or 37, where the multispecific antibody is administered at a dose of about 0.001 mg/kg to about 1 mg/kg.

39. The method of claim 1, wherein the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.

40. A method of transducing immune cells in a subject in need thereof, comprising:
a) administering a polynucleotide encoding a multispecific antibody to activate immune cells in the subject; and
b) administering a vector, optionally a viral vector;
wherein the method transduces the immune cells

41. The method of claim 40, wherein the polynucleotide encoding a multispecific antibody is an RNA.

42. The method of claim 40, wherein the immune cells are T cells.

43. The method of claim 40, wherein the vector is a lentiviral vector.

44. The method of claim 42, wherein the multispecific antibody comprises a T-cell antigen-specific binding domain.

45. The method of claim 44, wherein the T-cell antigen is CD3, CD4, CD8, or TCR.

46. The method of claim 44, wherein the multispecific antibody comprises a second antigen-specific binding domain.

47. The method of claim 46, wherein the second antigen is CD19.

48. The method of claim 46, wherein the second antigen is CD19, EpCAM, Her2/neu, EGFR, CD66e, CD33, EphA2, MCSP, CD22, CD79a, CD79b, or sIgM.

49. The method of claim 46, wherein the second antigen is CD19, EpCAM, CD20, CD123, BCMA, B7-H3, CDE, or PSMA.

50. The method of claim 46, wherein the second antigen is a lymph node antigen.

51. The method of claim 40, wherein the multispecific antibody is a trispecific antibody.

52. The method of claim 40, wherein the multispecific antibody is a bispecific antibody.

53. The method of claim 52, wherein the bispecific antibody is a bispecific T-cell engager (BiTE).

54. The method of claim 53, wherein the BiTE is a CD19×CD3 BiTE.

55. The method of claim 54, wherein the CD19×CD3 BiTE is blinatumomab.

56. The method of any one of claims 40-55, wherein the multispecific antibody activates the immune cells.

57. The method of any one of claims 40-55, wherein the multispecific antibody increases activation of the immune cells compared to administration of a vehicle control.

58. The method of any one of claims 40-55, wherein the multispecific antibody increases the number of immune cells in a lymph node of the subject.

59. The method of any one of claims 40-55, wherein the multispecific antibody increases transduction of the immune cells compared to administration of the viral vector alone.

60. The method of any one of claims 40-55, wherein the multispecific antibody enhances in vivo transduction of the immune cells by the viral vector.

61. The method of claim 60, wherein the multispecific antibody reduces the effective concentration (EC₅₀) of the viral vector.

62. The method of claim 60, wherein the method achieves the same level of transduction of immune cells as a method comprising administering a higher concentration of the viral vector without administering the multispecific antibody.

63. The method of claim 40, wherein step a) and/or step b) comprises subcutaneous administration.

64. The method of claim 40, wherein step a) and/or step b) comprises intralymphatic administration.

65. The method of claim 40, wherein step a) and/or step b) comprises intravenous administration.

66. The method of claim 40, wherein the viral vector comprises a polynucleotide encoding a T cell receptor or chimeric antigen receptor.

67. The method of claim 66, wherein the chimeric antigen receptor is an anti-CD19 chimeric antigen receptor.

68. The method of claim 40, wherein the viral vector comprises a polynucleotide encoding a cytokine receptor.

69. The method of claim 68, wherein the cytokine receptor is a drug-inducible cytokine receptor.

70. The method of claim 43, wherein the lentiviral vector comprises one or more cell surface receptors that bind to a ligand on a target host cell, heterologous viral envelope glycoproteins, fusion glycoproteins, T-cell activation or co-stimulation molecules, ligands for CD19 or a functional fragment thereof, cytokines or cytokine-based transduction enhancers, and/or transmembrane proteins comprising a mitogenic domain and/or cytokine-based domain exposed on the surface and/or conjugated to the surface of the lentiviral vector.

71. The method of claim 70, wherein one or more T-cell activation or co-stimulation molecules comprise one or more T-cell ligands.

72. The method of claim 40, wherein the vector further comprises one or more transgenes.

73. The method of claim 72, wherein the viral vector comprises the transgene encoding a TGFβ dominant negative receptor.

74. The method of claim 43, wherein the lentiviral vector is pseudotyped with a Cocal virus envelop protein.

75. The method of claim 43, wherein the lentiviral vector is pseudotyped with a Nipah virus envelop protein.

76. The method of claim 75, wherein the Nipah envelop protein is engineered to bind EpCAM, CD4, or CD8.

77. The method of claim 43, wherein the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.

78. A combination therapy for use in transducing immune cells in vivo, comprising a multispecific antibody and a vector, optionally a viral vector.

79. A pharmaceutical composition, comprising a multispecific antibody and a vector, optionally a viral vector.

80. A kit comprising 1) a multispecific antibody and 2) a vector, optionally a viral vector.

81. A kit comprising 1) a polynucleotide encoding a multispecific antibody and 2) a vector, optionally a viral vector.

82. The kit of claim 80 or 81, for use in:
a) transducing immune cells in a subject in need thereof; and/or
b) treating a disease or disorder in a subject in need thereof.

83. A method of treating a disease or disorder in a subject in need thereof, comprising:
a) administering a multispecific antibody to activate immune cells in the subject; and
b) before, after and concurrently with step a), administering a vector, optionally a viral vector.

84. The method of claim 83, wherein the method transduces the immune cells.

85. The method of claim 83 or 84, wherein the disease or disorder is a cancer.

86. The method of claim 83 or 84, wherein the disease or disorder is a hematological malignancy.

87. The method of claim 86, wherein the hematological malignancy is B cell lymphoma.

88. The method of claim 85, wherein the method treats the disease or disorder faster than administering the multispecific antibody alone and/or the vector alone.

89. The method of claim 85, wherein the method results in a better treatment outcome of the disease or disorder than administering the multispecific antibody alone and/or the vector alone.

90. The method of claim 85, wherein the multispecific antibody binds specifically to CD3 and CD19, wherein the vector is a lentiviral vector pseudotyped with a Cocal virus envelop protein, and wherein the vector comprises a polynucleotide encoding an anti-CD19 chimeric antigen receptor and a transgene encoding a TGFβ dominant negative receptor.

91. The method of claim 87, wherein the method results in faster depletion of malignant B cells in the subject than administering the multispecific antibody alone and/or the vector alone.

92. The method of claim 87, wherein the method results in lower number of residual malignant B cells and/or lower recurrence rate of the B cell lymphoma in the subject than administering the multispecific antibody alone and/or the vector alone.
